(19) United States
(12) Patent Application Publication
(10) Pub. No.: US 2010/0150839 A1
(43) Pub. Date: Jun. 17, 2010

(54) Compositions and Methods for Modulating Cognitive Function

(75) Inventor: Raymond Kelleher, Boston, MA (US)

Correspondence Address: CHOATE, HALL & STEWART LLP, TWO INTERNATIONAL PLACE, BOSTON, MA, 02110, US

(73) Assignee: Massachusetts Institute of Technology, Cambridge, MA (US)

(21) Appl. No.: 11/815,530

(22) PCT Filed: Feb. 6, 2006
(86) PCT No.: PCT/US06/04261

§ 371 Date: Aug. 3, 2007

Related U.S. Application Data
(60) Provisional application No. 60/649,978, filed on Feb. 4, 2005.

(51) Int. Cl. A61K 38/17; A61K 31/7088; C12Q 1/68; A61K 49/00; C12Q 1/48; A61P 25/28

(52) U.S. Cl. 424/9.2; 514/12; 514/44 A; 435/6; 435/15

(57) ABSTRACT

The present invention provides compositions and methods for modulating, e.g., enhancing, cognitive function. The methods involve modulation of translation, modulation of a MAPK signaling pathway, or both. The invention further provides screening methods useful in identifying compounds that modulate, e.g, enhance, cognitive function. The invention further provides method of treating a subject to modulate cognitive function.

### CROSS-REFERENCE TO RELATED APPLICATION

This application claims the benefit of, and priority to, U.S. Provisional Patent Application 60/649,978, filed Feb. 4, 2005, which is hereby incorporated herein by reference.

### GOVERNMENT SUPPORT

This invention was made with Government Support. The Government has certain rights in the invention.

### BACKGROUND OF THE INVENTION

A significant amount of research has been directed towards understanding the mechanisms involved in learning and memory. Increased understanding of these mechanisms, and ability to manipulate them, would be of extraordinary scientific interest and practical importance. Cognitive impairment associated with memory loss is one of the most common conditions that occurs in the elderly population, and a variety of disorders featuring impaired learning and/or memory formation occur in younger persons. Such impairment may include a reduction in the ability to learn new information and/or to retrieve information that has previously been learned. While memory loss has frequently been considered to be an aspect of normal aging, it is also a key feature of Alzheimer's disease (AD), a debilitating condition that affects an estimated 1.9 to 4 million persons in the United States (Clark 2003).

At present there is a lack of effective pharmacological methods for enhancing memory formation, for preventing the decline in memory that typically occurs with aging, or for treating other forms of cognitive impairment and/or memory loss such as amnesia that may occur as a result of trauma or damage due to stroke, tumors, etc. Current therapies for Alzheimer's disease include acetylcholinesterase inhibitors such as donepezil, rivastagmine, and galantamine. However, these drugs provide only modest benefit in improvement of symptoms, and there is little evidence to suggest efficacy in terms of slowing progression of the disease.

It is evident that a considerable need exists in the art for identification of compounds that can enhance memory formation and for identification of compounds that can prevent and/or treat cognitive dysfunction and/or memory impairment. There also exists a need in the art for new screening strategies to identify such compounds.

### SUMMARY OF THE INVENTION

The present invention provides a new understanding of the molecular basis of memory formation and synaptic plasticity

In one aspect, the invention provides a method of modulating cognitive function in a mammalian subject comprising steps of: (i) providing a mammalian subject in need of modulation of cognitive function; and (ii) administering to the subject a composition comprising an agent that modulates translation.

The invention further provides a method of modulating cognitive function in a mammalian subject comprising steps of: (i) providing a mammalian subject in need of modulation of cognitive function; and (ii) administering to the subject a composition that modulates a MAPK pathway.

The invention also provides a method of modulating cognitive function in a mammalian subject comprising steps of: (i) providing a mammalian subject in need of modulation of cognitive function; and (ii) administering to the subject a composition that modulates the mTOR pathway.

Compositions of the invention may consist of a single active agent, optionally with a pharmaceutically acceptable carrier and/or excipients, other formulation components, etc., or may consist of multiple active agents, optionally together with a pharmaceutically acceptable carrier and/or excipients, other formulation components, etc. The methods of the invention may be advantageously employed to enhance cognition and/or memory. In certain embodiments of the invention the agents are administered to a subject suffering from or at risk of a disease, disorder, or condition selected from the group consisting of: benign senescent forgetfulness, age-associated memory impairment, age-associated cognitive decline, mild cognitive impairment, Alzheimer's disease, dementia due to any of a variety of causes, trauma-associated cognitive impairment, toxin-associated cognitive impairment, stroke, etc. In certain embodiments of the invention the agents are administered to a subject suffering from or at risk of a disease, disorder, or condition selected from the group consisting of: mental retardation, fragile X syndrome, tuberous sclerosis, and autism. In any of the embodiments of the invention neuronal stimulation can be employed in conjuction with administration of a composition of the invention.

In another aspect the invention provides method of modulating translation in a neuron comprising contacting the neuron with an agent that modulates expression or activity of a component of the MAPK signaling pathway. In one embodiment the agent enhances or activates expression or activity of a component of the MAPK signaling pathway so that translation is increased. In another embodiment the agent inhibits or represses expression or activity of a component of the MAPK signaling pathway so that translation is reduced. The contacting may take place in cell or tissue culture or may take place in a living mammalian organism (e.g., following administration of the agent to the subject.) In some embodiments the component of the MAPK signaling pathway is ERK.

Additional aspects of the invention provide a variety of methods for identifying modulators of the MAPK pathway, the general translation machinery, and/or the mTOR pathway. Such modulators are of use for modulating cognitive function.

Where proteins are referred to herein, it is to be understood that isoforms of such proteins are referred to unless otherwise indicated, though certain isoforms may be preferred. For example, ERK refers to ERK1 and/or ERK2. 4E-BP refers to 4E-BP1, 4E-BP2, 4E-BP3, or any subset thereof. An abbreviation such as ERK1/2 means ERK1 and ERK2. Splice variants are also encompassed.

Where molecules are referred to herein, it is to be understood that the protonation state of various atoms may differ depending on factors such as the pH, as will be understood by one of ordinary skill in the art. All ionized and nonionized forms are included in various embodiments of the invention, and the depiction of a molecule with particular atoms in a charged or uncharged, protonated or unprotonated state is not intended to indicate that the molecules are necessarily in such a state. Furthermore, prodrugs and salts of the compounds are included, as further discussed below.

Unless otherwise stated, molecules referred herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds described herein are within the scope of the invention. Additionally, unless otherwise stated, structures named herein are also meant to include compounds that differ only in the presence of one or more isotopically enriched atoms. For example, compounds in which hydrogen has been replaced by deuterium or tritium, carbon has been replaced by a ¹³C- or ¹⁴C-enriched carbon, the replacement of nitrogen, phosphorus, or sulfur with an isotope thereof, etc., are within the scope of this invention. Such compounds are useful, for example, as analytical tools or probes in biological assays.

This application refers to various patents and publications. The contents of all of these are incorporated by reference. In addition, the following publications are incorporated herein by reference: Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Kandel, E., Schwartz, J. H., Jessell, T. M., (eds.), Principles of Neural Science, 4^{th} ed., McGraw Hill, 2000; Cowan, W. M., Südhof, T. C., and Stevens, C. F., (eds.); Hershey, J W, et al., (eds), Translational Control, Cold Spring Harbor Laboratory Press, Cold Spring Harbor, 1996; Sonenberg, N, et al. (eds.) Translational Control of Gene Expression, Cold Spring Harbor Laboratory Press, Cold Spring Harbor, 2000; Nagy, A., et al., Manipulating the Mouse Embryo: A Laboratory Manual, 3^{rd} ed., Cold Spring Harbor Laboratory Press, Cold Spring Harbor, 2003; Goodman and Gilman's The Pharmacological Basis of Therapeutics, 10^{th} Ed. McGraw Hill, 2001 (referred to herein as Goodman and Gilman). In case of a conflict between the instant specification and one or more of the incorporated references, the specification shall control. The determination of whether a conflict exists can be made by the inventors at any time.

Unless otherwise stated, the invention employs standard methods of behavioral testing, cell biology, cell culture, immunology, microbiology, molecular biology, transgenic biology, recombinant DNA technology, and formulation and administration of therapeutic agents. The afore-mentioned references describe exemplary methods in certain of these areas.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

### BRIEF DESCRIPTION OF THE DRAWING

FIG. 1. Generation of forebrain-specific dnMEK1 mice. (A) The construct used to produce single transgenic mice bearing a floxed stop cassette is depicted at top. When these single transgenic mice are crossed to αCaMKII-Cre transgenic mice, the stop cassette is excised and the dnMEK1 cDNA is expressed only in the postnatal forebrain of the resulting double transgenic mice (depicted at bottom). (B) Representative results of in situ hybridization with a transgene-specific probe are shown for the single transgenic “floxed” mice (left) and double transgenic “dnMEK1” mice (right) diagrammed above. (C) Neuronal activity-dependent ERK activation is impaired in the hippocampus of dnMEK1 mice. Top, representative Western analysis using antisera directed against dually-phosphorylated ERK and total ERK. Hippocampal slices from control and mutant mice were treated with aCSF alone or aCSF containing 90 mM KCl. Bottom, quantification of mean normalized levels of pERK (n=4 each).

FIG. 2. Impaired hippocampal memory consolidation in dnMEK1 transgenic mice. (A) Escape latencies in the hidden platform version of the Morris water maze are plotted as a function of training block number for control (n=20) and dnMEK1 (n=12) mice. (B) Mice were subjected to probe trials after the completion of training, and the mean proportion of time spent in each of the training quadrants is presented for both groups. AL, adjacent left quadrant; T, target quadrant; AR, adjacent right quadrant; OP, opposite quadrant. (C) The mean number of crossings of the platform location during the probe trial is shown for the target quadrant and the corresponding locations in other quadrants. (D) Mice were subjected to contextual fear conditioning, and context tests were administered after retention delays of one hour and 24 hours. The mean percentage of time spent freezing during the context tests is presented for control (n=24) and dnMEK1 (n=12) groups. (E) Fecal boli were quantified as an independent measure of conditioned fear during the same context tests. (F) Independent groups of control and mutant mice were subjected to cued fear conditioning. Noncontextual memory for the experimental tone was assessed in a novel chamber after a retention delay of 48 hours. The mean percentage of time spent freezing prior to presentation of the tone (“pre-CS”) and during phasic presentation of the tone (“CS”) is shown for control (n=24) and dnMEK1 (n=12) groups. Statistically-significant differences at 95% confidence levels are denoted by asterisks.

FIG. 3. Impairment of the translation-dependent, transcription-independent phase of hippocampal L-LTP in dnMEK1 transgenic mice. (A) Normal basal synaptic transmission in dnMEK1 mice. The synaptic input-output curve shows fEPSP slopes as a function of fiber-volley amplitudes for control (n=16 slices, 12 mice) and mutant (16 slices, 9 mice) slices. (B) Normal paired-pulse facilitation in dnMEK1 mice. The facilitation ratio is shown as a function of interpulse interval for control (n=12 slices, 8 mice) and mutant (n=15 slices, 9 mice) slices. (C) Normal E-LTP in dnMEK1 mice. E-LTP was induced in control (n=8 slices, 8 mice) and mutant (n=8 slices, 8 mice) slices with two tetanic trains (100 Hz, 1 sec.) separated by 20 seconds. (D) Impaired L-LTP in dnMEK1 mice. L-LTP was induced in control (n=10 slices, 10 mice) and mutant (n=10 slices, 10 mice) slices with four tetanic trains (100 Hz, 1 sec.) separated by 5 minutes each. The inset traces at top show the fEPSP responses immediately prior to and 200 minutes after tetanization for control (left) and mutant (right) slices. (E) Actinomycin-D and anisomycin produce patterns of L-LTP inhibition with distinct kinetic profiles. L-LTP was induced in slices from control mice in the presence (n=8 slices, 8 mice) and absence (n=8 slices, 8 mice) of actinomycin-D and anisomycin. (F) The inhibitory effects of actinomycin-D and anisomycin treatment are occluded in dnMEK1 slices. L-LTP was induced in slices from dnMEK1 mice in the presence (n=8 slices, 8 mice) and absence (n=8 slices, 8 mice) of actinomycin-D and anisomycin. (G) The effects of anisomycin on L-LTP in control mice are indistinguishable from the effects of dnMEK1 transgene expression in mutant mice. Superimposition of the L-LTP results for anisomycin-treated control slices and untreated mutant slices is shown. (H) Quantification of the L-LTP magnitude at 60 minutes post-tetanization is shown for each group. L-LTP magnitude in dnMEK1 slices is significantly lower than that in untreated and actinomycin-D-treated control slices, but is indistinguishable from that in anisomycin-treated control slices.

FIG. 4. The ERK signaling pathway regulates neuronal activity-dependent translation of reporter mRNAs through a polyadenylation-independent mechanism. (A) Reporter mRNA translation is stimulated by increasing poly(A) tail lengths. Representative fluorescent images (10× view) show EGFP expression under conditions of spontaneous neuronal activity as a function of increasing mRNA poly(A) tail length (results with 0, 20, 60 and >150 residues are shown). (B) Reporter mRNA translation is neuronal activity- and ERK-dependent. The effects of the indicated pharmacologic agents on reporter mRNA translation under conditions of spontaneous activity are shown. Reporter expression levels are normalized to the expression level in the presence of U0126. (C-D) Reporter mRNA translation is stimulated in an ERK-dependent manner by multiple forms of neuronal activity. In C, examples of the stimulation of reporter mRNA translation by neuronal activity are shown (20× view). In D, reporter expression levels are normalized to the expression level in the presence of U0126 and in the absence of externally-added stimulants. (E-F) ERK-dependent stimulation of reporter mRNA translation does not require the CPEs. In E, examples of translational stimulation of reporter mRNA bearing mutations in both CPEs are shown. In F, reporter expression levels are normalized to the expression level in the presence of U0126 and in the absence of externally-added stimulants. (G) ERK-dependent translational stimulation of reporter mRNA does not require the hexamer (AAUAAA) sequence. Left, translational stimulation of reporter mRNA containing mutant hexamer and intact CPE sequences. Right, translational stimulation of reporter mRNA containing mutant hexamer and mutant CPE sequences. Reporter expression levels are normalized to the expression level in the presence of U0126. (H) In situ hybridization revealed no differences in neuronal survival or reporter mRNA stability, transfection efficiency or localization under the indicated conditions. Representative fluorescent images are shown at left. Relative mRNA levels are quantified at right. Statistically significant differences at 95% confidence levels are denoted by asterisks.

FIG. 5: The ERK signaling pathway regulates neuronal activity-dependent translation by modulating the phosphorylation state of translation initiation factors. (A) Stimulation of hippocampal neurons enhances ³⁵S-methionine incorporation and phosphorylation of ERK, S6, eIF4E and 4E-BP in an ERK-dependent manner. Representative autoradiogram shows that synthesis of all detectable protein species changes uniformly upon pharmacological treatment. (B) Quantification of ³⁵S-methionine incorporation in hippocampal neurons upon pharmacological treatment. (C—F) Quantification of normalized levels of phosphorylated ERK, S6, eIF4E and 4E-BP in hippocampal neurons upon pharmacological treatment. (G) Top, representative autoradiogram shows that the synthesis of all detectable protein species in synaptoneurosomes prepared from hippocampal neurons changes uniformly upon pharmacological treatment. Bottom, phosphorylation of ERK, S6 and eIF4E also occurs in an ERK-dependent manner. (H) Quantification of ³⁵S-methionine incorporation into synaptoneurosomes upon pharmacological treatment. (1-K) Quantification of normalized levels of phosphorylated ERK, S6 and eIF4E in synaptoneurosomes upon pharmacological treatment. In all panels, results are expressed relative to values obtained under conditions of spontaneous activity. Statistically significant differences at 95% confidence levels are denoted by asterisks.

FIG. 6: Stimulation of translational activity by L-LTP-inducing tetanization and long-term memory formation is impaired in dnMEK1 mice. (A) L-LTP-inducing tetanization stimulates protein synthesis in area CA1 of control but not dnMEK1 hippocampal slices. In contrast, tetanization stimulates similar levels of translation in area CA3 of control and dnMEK1 slices. Levels of ³⁵S-methionine incorporation following L-LTP induction are normalized to the levels in untetanized slices in paired experiments. (B-D) L-LTP-inducing tetanization stimulates phosphorylation of ERK, S6 and eIF4E in area CA1 of control but not dnMEK1 hippocampal slices. In contrast, L-LTP stimulates similar levels of phosphorylation of the same proteins in area CA3 of both control and dnMEK1 mice. Normalized levels of the indicated phosphoproteins are expressed relative to the corresponding levels in untetanized slices. (E-G) Phosphorylation of ERK, S6 and eIF4E induced by contextual fear conditioning is inhibited in dnMEK1 mice. Normalized levels of the indicated phosphoproteins are expressed relative to the corresponding levels in untrained control animals. Statistically significant differences at 95% confidence levels are denoted by asterisks.

FIG. 7: The molecular mechanism of translational regulation by the ERK signaling pathway. The present study addresses two possible mechanisms by which ERK signaling may regulate neuronal activity-dependent translation. (A) Inducible cytoplasmic polyadenylation has been proposed as a mechanism for stimulation of translational efficiency in response to neuronal activity (Richter and Lorenz, 2002). Cytoplasmic polyadenylation elements (CPE) in the distal 3′ UTRs of specific mRNAs (e.g. αCaMKII) are recognized by a specific binding protein, CPEB. CPEB phosphorylation in response to neuronal activity is proposed to result in polyadenylation, displacement of Maskin and poly(A)-binding protein (PABP)-mediated recruitment of eIF4G. In our study, activity-induced translation was strongly ERK-dependent in both the presence and absence of functional CPE (and hexamer) sequences, indicating that ERK regulates translation through a polyadenylation-independent mechanism. (B) Recognition of the mRNA 5′ cap by eIF4E and subsequent recruitment of the 40S ribosomal subunit by eIF4G are key steps in the initiation of translation. Phosphorylation of eIF4E and its inhibitor, 4E-BP, regulates the activity and availability of eIF4E for cap recognition and interaction with eIF4G. Increasing poly(A) tail lengths are thought to stimulate translation (see FIG. 4A) through an interaction of PABP with eIF4G. Our results demonstrate a general requirement for ERK signaling in neuronal activity-dependent translation. Consistent with these findings, phosphorylation of eIF4E, 4E-BP and S6 was stimulated by neuronal activity in a highly ERK-dependent manner. A similar ERK requirement for eIF4E and S6 phosphorylation was observed during hippocampal L-LTP and memory formation. Thus, the ERK pathway plays an important role in neuronal activity-dependent regulation of translation initiation.

FIG. 8: Stimulation of translation in hippocampal cultures by diverse agonists (A) Dopamine causes an increase in translation rate which is blocked by the MEK inhibitor U0126. (B) The mGluR1 agonist DHPG causes an increase in translation rate which is blocked by the MEK inhibitor U0126. (C) The mGluR1 agonist DHPG causes an increase in phosphorylation of eIF4E, which is blocked by the MEK inhibitor U0126. (D) The mGluR1 agonist DHPG causes an increase in phosphorylation of S6, which is blocked by the MEK inhibitor U0126.

FIG. 9: Bar graph showing that multiple neuromodulatory agents upregulate translation in hippocampal slices.

FIG. 10: Bar graph showing translational stimulation through administration of agonists of neuromodulatory receptors.

### DETAILED DESCRIPTION OF CERTAIN EMBODIMENTS OF THE INVENTION

### I. Overview

The present invention provides methods and compositions for modulating cognitive function, including but not limited to, long-term memory, in a mammalian subject. Considerable research has indicated that memory formation can be divided into two general phases. Storage of long-term memory, or memory consolidation, requires new protein synthesis (Davis and Squire, 1984; McGaugh, 2000). In contrast, short-term memory is insensitive to inhibitors of translation. Long-lasting forms of synaptic plasticity, such as “late LTP” (L-LTP), exhibit a similar dependence on macromolecular synthesis, whereas more transient modifications of synaptic strength, such as “early LTP” (E-LTP), can be established in the absence of new mRNA and protein synthesis (Kandel, 2001). Investigation of the molecular mechanisms underlying long-term memory and synaptic plasticity has largely focused on the roles of signal transduction to the nucleus and regulation of gene expression at the transcriptional level. Conversely, the possibility that regulation at the translational level may play a role in these processes has not heretofore been explored. Similarly, the possibility that intervening in translational regulation, i.e., modulating translation, could prove of benefit in modulating cognition has not received consideration.

The present invention arose from the inventors' discovery that translational control by MAP kinase (MAPK) signaling plays a crucial role in long-lasting forms of synaptic plasticity and memory and that ERK-dependent translational modulation in neurons is a general rather than a gene-specific phenomenon. The invention is further based on the identification of certain relevant targets of the ERK pathway in the general translational machinery. These discoveries provide the foundation for new approaches to modulating memory formation and cognition and for the treatment of diseases, disorders, and conditions in which there is an impairment or deficiency in one or more aspects of memory and/or cognitive function. To the best of the inventors' knowledge, this work represents the first study to show that ERK regulates translation during hippocampal synaptic plasticity and memory.

One aspect of the present invention is a method for modulating memory or cognition in a mammalian subject comprising: (i) providing a mammalian subject in need of modulation of cognitive function; and (ii) administering to the subject a composition comprising an agent that modulates translation. In certain preferred embodiments the modulation is non-gene specific. In certain preferred embodiments the agent increases or decreases activity or abundance of a component of the general translation machinery. The invention includes a variety of methods for intervening in translational regulation, based at least in part on a knowledge of various components that play a role in translation and their corresponding activities, which are further discussed below.

Another aspect of the invention is a method of modulating cognitive function in a mammalian subject comprising steps of: (i) providing a mammalian subject in need of modulation of cognitive function; and (ii) administering to the subject a composition that modulates a MAPK pathway. Preferably the MAPK is an ERK, e.g., ERK1 or ERK2. The invention includes a variety of methods for intervening in a MAPK pathway based at least in part on a knowledge of its various components and their activities, which are further discussed below.

According to certain embodiments of the above methods the composition comprises an agent selected from the group consisting of: a tyrosine kinase receptor agonist, a G protein coupled receptor agonist; a metabotropic glutamate receptor agonist; an NMDA receptor agonist; a GABA receptor antagonist (e.g., a GABA_{A} or GABA_{B} receptor antagonist); an ERK pathway activator; an adenylyl cyclase activator; a protein kinase A activator; a phosphodiesterase inhibitor, a dopamine receptor agonist, a noradrenergic receptor agonist (e.g., a β-adrenergic receptor agonist), or a muscarinic acetylcholine receptor agonist. In certain embodiments of the invention a composition comprising one or more of these agents enhances cognitive function, e.g., in a subject suffering from age-associated cognitive decline, mild cognitive impairment, Alzheimer's disease, dementia due to any of a variety of causes, trauma-associated cognitive impairment, toxin-associated cognitive impairment, etc. The subject may be, e.g., at least about 50 years of age. The inventors have demonstrated induction of MAPK-dependent protein synthesis in neurons by, e.g., agonists of dopamine, noradrenergic and/or muscarinic acetylcholine receptors.

In certain embodiments of the invention the composition comprises an agent selected from the group consisting of: a tyrosine kinase receptor antagonist, a G protein coupled receptor antagonist; a metabotropic glutamate receptor antagonist; an NMDA receptor antagonist; a GABA receptor agonist (e.g., a GABA_{A} or GABA_{B} receptor agonist); an ERK pathway inhibitor; an adenylyl cyclase inhibitor; a protein kinase A inhibitor; and a phosphodiesterase activator, a dopamine receptor agonist, a noradrenergic receptor antagonist (e.g., a β-adrenergic receptor antagonist), or a muscarinic acetylcholine receptor antagonist. In certain embodiments a composition comprising one or more of these agents reduces cognitive function, e.g., in an individual with normal cognitive function. In certain embodiments a composition comprising one or more of these agents enhances cognitive function, e.g., in a subject in whom excessive protein synthesis occurs, e.g., a subject suffering from or at risk of mental retardation due to various causes, fragile X syndrome, tuberous sclerosis, or autism.

Another aspect of the invention is a variety of screening methods to identify agents that modulate translation in vitro and/or in vivo (i.e., in animals). Preferably the compounds modulate translation in neurons. The agents identified using these screening methods may be used to modulate memory, cognition, learning, etc., in a subject. Preferred compounds enhance one or more aspects of cognition. The compounds may thus be used for the treatment and/or prevention of diseases and conditions associated with memory loss, cognitive impairment, and the like. Certain compounds reduce one or more aspects of cognition. Reducing a cognitive function may be desirable, for example, when a subject is expected to experience an event that he or she does not wish to remember, e.g., a painful, embarrassing, or stressful event. By reducing the ability to form long-term memories prior to the event, the subject may avoid forming memories that will subsequently be unpleasant to recall.

### II. Definitions

The term “agonist” generally refers to a substance that can directly interact with (e.g, bind to) a receptor and initiate a physiological or a pharmacological response characteristic of the activity of that receptor, e.g., the activity that is normally induced by interaction of an endogenous positively-acting ligand with the receptor. Substances generally recognized in the literature as agonists of a particular receptor are of use in the methods described herein. The term “agonist” also refers to partial agonists, i.e., compounds that are capable of partially activating a receptor, e.g., activating it to a lesser extent than its endogenous ligand. The term also encompasses substances that indirectly stimulate a receptor, e.g., by inhibiting reuptake or breakdown/metabolism of an endogenous direct agonist and/or by stimulating the production or release of an endogenous direct agonist.

The term “antagonist” generally refers to a substance that opposes the receptor-associated responses normally induced by another bioactive agent such as an endogenous positively-acting ligand. Typically, an antagonist binds to a receptor and prevents binding of an endogenous ligand that would normally activate the receptor, or prevents binding of an exogenous agonist to the receptor. The antagonist may or may not induce an effect itself. The activity of a receptor is generally taken to be the activity associated with binding of an endogenous positively-acting ligand. Substances generally recognized in the literature as antagonists of a particular receptor are of use in the methods described herein. The term also encompasses substances that indirectly inhibit a receptor, e.g., by inhibiting reuptake or by stimulating breakdown/metabolism of an endogenous direct agonist and/or by stimulating the production or release of an endogenous direct antagonist.

“Approximately” or “about” in reference to a number includes numbers that fall within a range of 5% in either direction (greater than or less than) the number unless otherwise stated or otherwise evident from the context (except where such number would exceed 100% of a possible value).

“Biological pathway” refers to a sequence of reactions (e.g., physical interactions between molecules, enzyme-substrate reactions) that takes place in a living organism, typically resulting in a biological effect. A pathway typically involves a cascade of events in which multiple components of the pathway signal to each other, often in a characteristic and ordered manner. In some cases some or all of the events of a pathway can be recapitulated in a cell-free system. Biological pathways of interest herein include the MAP kinase pathway and specific subpathways such as the ERK pathway and the mTOR translational regulation pathway.

“Biological system” refers to any system containing at least one biological component, e.g., a biological macromolecule such as a protein or nucleic acid, suitable for performing an assay of a biological function or activity. The term includes cell-free systems, cells, collections of cells, animals, etc.

“Cognition” generally refers to the process of obtaining, organizing, and using knowledge. Enhancing cognitive function refers to enhancing any aspect of this process, e.g., learning, the performance of mental operations, the storage and/or retrieval of information or thoughts (e.g., memory), and/or preventing a decline from a subject's current state. Long-term memory is thought to involve steps of registration, rehearsal, and retention of information. In a human subject, long-term memory refers to memory lasting at least 24 hours after the event to be remembered. Numerous standardized tests can be used to evaluate cognitive function. Such tests can be used to identify subjects in need of enhancement of cognitive function and/or to monitor the effects of treatment. Suitable tests include, but are not limited to, the Mini-Mental Status Exam (Folstein, 1975), components of the PROSPER neuropsychological test battery (Houx, 2002), etc. Family history, age, and other factors may also be used to identify subjects in need of enhancement of cognitive function.

“Complementary” is used herein in accordance with its art-accepted meaning to refer to the capacity for precise pairing between particular bases, nucleosides, nucleotides or nucleic acids via formation of hydrogen bonds. For example, adenine (A) and uridine (U), adenine (A) and thymidine (T), or guanine (G) and cytosine (C), are complementary to one another. If a nucleotide at a certain position of a first nucleic acid is complementary to a nucleotide located opposite in a second nucleic acid, the nucleotides form a complementary base pair, and the nucleic acids are complementary at that position. One of ordinary skill in the art will appreciate that the nucleic acids are aligned in antiparallel orientation (i.e., one nucleic acid is in 5′ to 3′ orientation while the other is in 3′ to 5′ orientation). “Complementary” sequences as used herein refer to sequences which have sufficient complementarity to be able to hybridize, forming a stable duplex under the conditions of interest.

“Concurrent administration” as used herein with respect to two or more agents, e.g., therapeutic agents, is administration performed using doses and time intervals such that the administered agents are present together within the body, or at a site of action in the body such as in the CNS) over a time interval in less than de minimis quantities. The time interval can be minutes, hours, days, weeks, etc. Accordingly, the agents may, but need not be, administered together as part of a single composition. In addition, the agents may, but need not be, administered simultaneously (e.g., within less than 5 minutes, or within less than 1 minute) or within a short time of one another (e.g., less than 1 hour, less than 30 minutes, less than 10 minutes, approximately 5 minutes apart). According to various embodiments of the invention agents administered within such time intervals may be considered to be administered at substantially the same time. One of ordinary skill in the art will be able to readily determine appropriate doses and time interval between administration of the agents so that they will each be present at more than de minimis levels within the body or, preferably, at effective concentrations within the body. For example, a de minimis concentration of an agent in a particular body compartment (e.g., the blood, the CSF, etc.) may be less than about 0.1 times its effective concentration. When administered concurrently, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

An “effective amount” of an active agent refers to the amount of the active agent sufficient to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. A desired biological response may be, for example, (i) an increase in synaptic plasticity; (ii) an improvement in a task requiring cognitive function, e.g., improved performance on a test that measures learning and/or memory; (iii) a slowing in the rate of decline in cognitive function, e.g., as measured by performance on a test that measures learning and/or memory.

“Enhancing”, as used herein in reference to cognitive function, includes increasing, augmenting, improving, reducing loss or decline of, etc.

“Gene”, as used herein, has its meaning as understood in the art. In general, a gene may include gene regulatory sequences (e.g., promoters, enhancers, etc.) and/or intron sequences, in addition to coding sequences (open reading frames). It will further be appreciated that definitions of “gene” include references to nucleic acids that do not encode proteins but rather encode functional RNA molecules such as tRNAs. For the purpose of clarity it is noted that, as used in the present application, the term “gene” generally refers to a portion of a nucleic acid that encodes a protein, optionally encompassing regulatory sequence(s). This definition is not intended to exclude application of the term “gene” to non-protein coding expression units but rather to clarify that, in most cases, the term as used in this document refers to a nucleic acid that encodes a protein.

A “gene product” or “expression product” is, in general, an RNA transcribed from the gene (e.g., either pre- or post-processing) or a polypeptide encoded by an RNA transcribed from the gene (e.g., either pre- or post-modification).

“Interact” as used herein in reference to molecules, etc., refers to direct physical interactions and also refers to the ability of a first molecule to influence the activity or abundance of a second molecule, whether such influence is exerted by a direct physical interaction with the second molecule or otherwise (e.g., by generating a “second messenger” that itself physically interacts with the second molecule, by altering a third molecule that itself physically interacts with the second molecule, etc.). If RNA transcripts and/or proteins interact with one another (e.g., RNA/RNA interaction, RNA/protein interaction, or protein/protein interaction), then the genes encoding such RNA transcripts and/or proteins are said to interact.

“Isolated”, as used herein, means 1) separated from at least some of the components with which it is usually associated in nature; 2) prepared or purified by a process that involves the hand of man; and/or 3) not occurring in nature.

“Long-term memory” refers to memory formation that requires new protein synthesis, wherein the memory persists for at least 24 hours.

“Long-term potentiation” (LTP) refers to a property of the nervous system or a neuronal culture in which a brief, high frequency stimulus pattern increases the amplitude of subsequent excitatory postsynaptic potentials in the target neurons. LTP is known to exist in a variety of pathways in the CNS, including three major pathways in the hippocampus. LTP can be induced in laboratory studies in neuronal cultures or intact brain slices by application of a high frequency stimulus or by directly depolarizing postsynaptic cells while maintaining low frequency stimulation. One widely applied technique for inducing LTP is referred to as theta-burst stimulation, which mimics stimulation patterns known to occur in the hippocampus. LTP is a widely studied example of synaptic plasticity thought to be of major importance in learning and memory. L-LTP refers to the late phase of LTP, which requires new protein synthesis. E-LTP refers to the early phase of LTP, which does not require new protein synthesis.

“Modulate” means to increase, up-regulate, stimulate, enhance, etc., or to decrease, down-regulate, inhibit, diminish, etc.

“Non gene specific manner” in reference to modulating translation of an mRNA that is transcribed from the gene means that such modulation does not require the presence of specific regulatory element(s) in the mRNA and does not require that the mRNA is localized in a particular region of the cell. In certain embodiments non gene-specific modulation of translation results in a detectable change in translation (e.g., an increase or decrease of at least 5%) of at least 10%, at least 25%, at least 50%, at least 75%, at least 90%, etc. of mRNA species in a cell.

“Operably linked” or “operably associated” refers to a relationship between two nucleic acid sequences wherein the expression of one of the nucleic acid sequences is controlled by, regulated by, modulated by, etc., the other nucleic acid sequence, or a relationship between two polypeptides wherein the expression of one of the polypeptides is controlled by, regulated by, modulated by, etc., the other polypeptide. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport, stability, or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence, or a polypeptide that is operatively linked to a second polypeptide, is covalently linked, either directly or indirectly, to such a sequence, although any effective three-dimensional association is acceptable.

“Percent (%) amino acid sequence identity” with respect to the amino acid sequences of polypeptides discussed herein is defined as the percentage of amino acid residues in a polypeptide sequence that are identical with the amino acid residues in the specific polypeptide sequence of interest after aligning the sequences and introducing gaps, as needed, to achieve the maximum percent sequence identity. Alignment can be performed in various ways known to those of skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. US Publication No. 20030211568 describes a number of suitable methods. For example, as described therein, % amino acid sequence identity values may be obtained as follows using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the polypeptide of interest and the comparison amino acid sequence of interest (i.e., the sequence against which the polypeptide of interest is being compared for purposes of determining % identity) as determined by WU-BLAST-2 by (b) the total number of amino acid residues of the polypeptide of interest. For example, in the statement “a polypeptide comprising an the amino acid sequence A which has or having at least 80% amino acid sequence identity to the amino acid sequence B”, the amino acid sequence A is the comparison amino acid sequence of interest and the amino acid sequence B is the amino acid sequence of the polypeptide of interest. Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov. NCBI-BLAST2 uses several search parameters, wherein all of those search parameters are set to default values including, for example, unmask=yes, strand all, expected occurrences=10, minimum low complexity length=1515, multi-pass e-value=0.01, constant for multi-pass=25, dropoff for final gapped alignment=25 and scoring matrix=BLOSUM62. In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Physical interaction” as used herein, includes detectable physical interactions between molecules, e.g., interactions that can be detected using, for example, a two hybrid assay, three hybrid assay, radioligand binding assay, immunoassay, gel shift assay, etc.

“Polynucleotide” or “oligonucleotide” refers to a polymer of nucleotides. As used herein, an oligonucleotide is typically less than 100 nucleotides in length. A polynucleotide is also referred to as a nucleic acid. Naturally occurring nucleic acids include DNA and RNA. The polymer may include natural nucleosides (e.g., adenosine, thymidine, guanosine, cytidine, uridine, deoxyadenosine, deoxythymidine, deoxyguanosine, and deoxycytidine), nucleoside analogs, etc. See U.S. Patent Application No. 20040092470 and references therein for further discussion of various nucleotides, nucleosides, and backbone structures that can be used. The polynucleotide may be double-stranded or single-stranded, and if single-stranded may be either a coding (sense) strand or non-coding (anti-sense) strand.

“Polypeptide”, as used herein, refers to a polymer of amino acids. A protein is a molecule composed of one or more polypeptides. A peptide is a relatively short polypeptide, typically between about 2 and 60 amino acids in length. The terms “protein”, “polypeptide”, and “peptide” may be used interchangeably. Peptide may refer to an individual peptide or a collection of peptides. Polypeptides used herein preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Positive receptor modulator” is used to refer to a compound that potentiates the ability of a receptor agonist to activate the receptor. In many instances the compound itself lacks intrinsic activity at the receptor. In some cases the compound itself may have some activity, but typically much less than that of the endogenous agonist. Examples include compounds that act as positive allosteric modulators, inhibitors of agonist metabolism to inactive compounds, inhibitors of agonist transport, etc.

“Purified”, as used herein, means separated from many other compounds or entities. A compound or entity may be partially purified, substantially purified, or pure, where it is pure when it is removed from substantially all other biological macromolecules (such as other polypeptides in the case of a polypeptide), i.e., is preferably at least about 90%, more preferably at least about 91%, 92%, 93%, 94%, 95%, 96%, 97%, 98%, 99%, or greater than 99% pure. In the context of a preparation of a polypeptide, a preparation may be considered substantially pure if the polypeptide represents at least 80%, more preferably at least 90%, yet more preferably at least 95% of total protein in a preparation. The percentages listed refer to dry weight.

“Recombinant” is used consistently with its use in the art and generally refers to a nucleic acid or polypeptide that contains sequences not normally found in nature and/or not normally found in a single molecule in nature (or not found in the order or configuration existing in the recombinant molecule) and that occur together in the recombinant molecule as a result of the hand of man, i.e., the hand of man was involved in creation of the nucleic acid or polypeptide or in creation of a precursor thereof. The term may also be applied to cells, organisms, etc., that contain and/or express a recombinant nucleic acid or polypeptide.

“Regulatory element” or “regulatory sequence” is generally used herein to describe a portion of nucleic acid that directs or influences one or more steps in the expression of nucleic acid sequence(s) with which it is operatively linked. The regulatory element may influence transcription, splicing, translation, polyadenylation, or other forms of post-transcriptional or post-translational processing. The term includes transcriptional control elements such as promoters and enhancers and also translational regulation elements such as cytoplasmic polyadenylation elements (CPEs) (see Richter, J. D. in Translational Control of Gene Expression, supra), hexamer sequences (e.g., AAUAAA), etc. The term encompasses any cis-acting sequence found in a subset of genes that influences translation of an operatively linked open-reading frame. Such elements are often located in the 5′ or 3′ UTR of an mRNA. Typically the regulatory activity of these sequences can be established by inserting the sequence into a reporter mRNA that encodes a readily detectable product (e.g., a fluorescent or chemiluminescent protein, enzyme, etc.) and comparing translation of the original reporter mRNA with the modified reporter mRNA. If addition of the sequence alters translation, the sequence is identified as a translational regulation element. Regulatory elements as used herein are distinct from sequences such as start codons, Kozak consensus sequences, etc., which are required for basal levels of translation of most or all eukaryotic mRNAs. Regulatory sequences may direct constitutive expression of a nucleotide sequence (e.g., expression in most or all cell types under typical physiological conditions in culture or in an organism); in other embodiments, regulatory sequences may direct cell or tissue-specific and/or inducible expression. For example, expression may be induced by the presence or addition of an inducing agent such as a hormone or other small molecule, by an increase in temperature, etc. Regulatory elements may also inhibit or decrease expression of an operatively linked nucleic acid. Regulatory elements that behave in this manner will be referred to herein as “negative regulatory elements” to distinguish them from regulatory elements that direct or increase expression.

In general, the level of expression may be determined using standard techniques for measuring mRNA or protein. Such methods include Northern blotting, in situ hybridization, RT-PCR, sequencing, immunological methods such as immunoblotting, immunodetection, or fluorescence detection following staining with fluorescently labeled antibodies, oligonucleotide or cDNA microarray or membrane array, protein array analysis, mass spectrometry, etc. A convenient way to determine expression level is to place a nucleic acid that encodes a readily detectable marker (e.g., a fluorescent or luminescent protein such as green fluorescent protein or luciferase, an enzyme such as alkaline phosphatase, etc.) in operable association with the regulatory element in an expression vector, introduce the vector into a cell type of interest or into an organism, maintain the cell or organism for a period of time, and then measure expression of the readily detectable marker, taking advantage of whatever property renders it readily detectable (e.g., fluorescence, luminescence, alteration of optical property of a substrate, etc.). Comparing expression in the absence and presence of the regulatory element indicates the degree to which the regulatory element affects expression of an operatively linked sequence.

“RNA interference” is used herein as understood in the art and refers to the sequence-specific silencing of gene expression by double-stranded RNA molecules. RNAi and agents capable of mediating RNAi are described in, e.g., Dykxhoorn, D M, et al., Nature Rev. Molecular Cell Biology, 4:457-467, 2003 and references cited therein and in US Publication Nos. 20030108923 and 20040259248. RNA interference is widely used in cell culture as a convenient method to rapidly and specifically reduce or eliminate expression of a target gene. RNA interference has also been used to silence gene expression in tissues of mammalian subjects either by administration of siRNAs or by expression of transgenes that encode shRNA molecules. RNAi is believed to function by at least two different pathways, one involving cleavage of a target transcript and the other involving translational repression. RNAi agents functioning via either of these pathways may be used to effect silencing of a desired target gene either in cell culture or in a subject in accordance with the present invention.

“RNAi agent” refers to a nucleic acid molecule such as a short interfering RNA (siRNA) or short hairpin RNA (shRNA) that inhibits expression of a target gene by RNAi. The term encompasses agents with duplexes having bulges and/or mismatches, precursors of siRNA or shRNA species, microRNA precursors, etc. See Dykxhoorn, D M, et al., Nature Rev. Molecular Cell Biology, 4:457-467, 2003 and references cited therein. Selection of appropriate siRNA and shRNA sequences can be performed according to guidelines well known in the art, e.g., taking factors such as desirable GC content into consideration. See, e.g., Ambion Technical Bulletion #506, available at the web site having URL www.ambion.com/techlib/tb/tb_{—}506.html. Following these guidelines approximately half of the selected siRNAs effectively silence the corresponding gene, indicating that by selecting about 5 siRNAs it will almost always be possible to identify an effective sequence. A number of computer programs that aid in the selection of effective siRNA/shRNA sequences are known in the art, which yield even higher percentages of effective siRNAs. See, e.g., Cui, W., et al., “OptiRNai, a Web-based Program to Select siRNA Sequences”, Proceedings of the IEEE Computer Society Conference on Bioinformatics, p. 433, 2003. Pre-designed siRNAs targeting over 95% of the mouse or human genome are commercially available, e.g, from Ambion and/or Cenix Biosciences. See web site having URL www.ambion.com/techlib/tn/104/5.html. Additional discussion of RNAi agents, e.g., siRNAs, are found in (Novina, C. D. and Sharp, P. A., “The RNAi revolution”, Nature, 430, 161-164, 2004) and references therein as well as U.S. Ser. No. 09/821,832 (U.S. Pub. No. 20020086356) and U.S. Ser. No. 10/832,248 (U.S. Pub. No. 20040229266). One of ordinary skill in the art will appreciate that RNAi agents may consist entirely of nucleotides such as those found naturally in RNA and/or DNA or may comprise any of a wide variety of nucleotide analogs or may differ in other ways from the structure of naturally occurring RNA and DNA. See, e.g., U.S. Pub. Nos. 20030175950, 20040192626, 20040092470, 20050020525, 20050032733. “RNAi vector” refers to a vector that comprises a template for transcription of an RNAi agent that inhibits expression of a target gene by RNAi. The template is operatively linked to expression signals sufficient for expression to occur in a cell or subject to which the vector is administered. RNAi vectors may be introduced into cells to confer on the cells a long-lasting or permanent ability to express an RNAi agent.

“Sequential administration” of two or more agents refers to administration of two or more agents to a subject such that the agents are not present together in the subject's body at greater than de minimis concentrations. Administration of the agents may, but need not, alternate. Each agent may be administered multiple times.

“Short hairpin RNA” refers to an RNAi-mediating RNA molecule that comprises a region that self-hybridizes to form a hairpin containing a stem and a loop. The stem is a duplex structure approximately 19-29 nucleotides in length, and the loop is typically between approximately 4 and 23 nucleotides in length. One portion of the molecule that participates in duplex formation comprises a region that is complementary, preferably 100% complementary, to a target transcript. There may be one or more mismatches or bulges in the duplex region. Short hairpin RNAs are believed to be processed intracellularly into siRNAs. A single shRNA may be processed to produce multiple distinct siRNA species.

“Short interfering RNA” refers to an RNAi-mediating short double-stranded RNA molecule comprising a duplex region typically approximately 19 nucleotides in length (but the length can vary between 17 and 29 nucleotides). Preferably the strands have 5′ phosphorylated ends and 2-nucleotide unphosphorylated 3′ ends. One strand of an siRNA (the “antisense” or “guide” strand) comprises a region (i.e., the region that participates in duplex formation) that is complementary, preferably 100% complementary, to a target transcript. There may be one or more mismatches or bulges in the duplex region.

“Small molecule”, as used herein, refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

“Subject”, as used herein, refers to an individual to whom an agent is to be delivered, e.g., for experimental, diagnostic, and/or therapeutic purposes. Preferred subjects are mammals, particularly domesticated mammals (e.g., dogs, cats, etc.), primates, or humans.

“Synaptic plasticity” is defined as the ability of a synapse to change its strength in response to a pattern of stimulation (i.e., one or more electrical or chemical stimuli), wherein the alteration in strength typically outlasts the event that triggers it. A synapse that exhibits this property is said to be plastic, or to display synaptic plasticity. A neural network in which some or all of the synapses exhibit plasticity is also said to exhibit synaptic plasticity.

A “transgene” means a nucleic acid that is partly or entirely heterologous, i.e., foreign, to the genome of a transgenic animal or cell into which it is introduced, or, is homologous to an endogenous gene of an transgenic animal or cell into which it is introduced, but which is designed to be inserted, or is inserted, into the genome in a manner that alters the genome of the cell into which it is inserted. For example, the nucleic acid may be inserted at a location which differs from that of the natural gene, or its insertion may result in a gene knockout. A transgene can include one or more regulatory sequences and any other nucleic acid, (e.g. an intron), that may be involved in regulating its expression. A transgene may comprise template(s) for transcription of one or more RNA interference (RNAi) agents such as a short hairpin RNA (shRNA), short interfering RNA (siRNA), etc., or a template for transcription of an antisense RNA or ribozyme.

A “transgenic animal” refers to any animal, preferably a non-human mammal such as a mouse, in which one or more of the cells of the animal contains a heterologous nucleic acid introduced by way of human intervention, e.g., by transgenic techniques well known in the art. The nucleic acid is introduced into the cell, directly or indirectly by introduction into a precursor of the cell, by way of deliberate genetic manipulation, such as by microinjection, by infection with a recombinant virus, etc. The term genetic manipulation does not include classical cross-breeding, or in vitro fertilization, but rather is directed to the introduction of a recombinant nucleic acid molecule. This molecule may be integrated within a chromosome, or it may be an extrachromosomally replicating nucleic acid. The transgene may cause cells to express a recombinant protein that differs in sequence from its naturally occurring counterpart. Certain of the transgenic animals described herein express “dominant negative” forms of a protein, which block or prevent activity of the endogenous form. However, the recombinant gene may be silent. Silent genes may be activatable, e.g., upon recombination. “Transgenic animals” also includes recombinant animals in which gene activity is inhibited by human intervention by means other than removal of all or a portion of the gene, or insertion of a heterologous nucleic acid into the gene. For example, gene activity may be inhibited by introduction of a construct that comprises template(s) for transcription of one or more RNAi agents such as an shRNA or siRNA or an antisense RNA or ribozyme. The construct may be expressed from an episome or may be intergrated into the genome of some or all cells of the organism.

“Treating”, as used herein, refers to administering a composition for prophylactic and/or therapeutic purposes. The term “prophylactic treatment” refers to treating a subject who is not yet detectably suffering from a disease, disorder, or condition but who is susceptible to, or otherwise at risk, of developing the disease, disorder, or condition. The term “therapeutic treatment” refers to administering treatment to a subject already suffering from a disease, disorder, or condition. With respect to a desired therapeutic effect in a subject such as a human being, treating can result in reversing, alleviating, inhibiting the progress of, preventing, or reducing the likelihood of the disease, disorder, or condition being treated, or one or more symptoms or manifestations of such disease, disorder or condition. “Preventing” refers to causing a disease, disorder, condition, or symptom or manifestation of such, or worsening of the severity of such, not to occur.

“Unit dosage form” as used herein refers to physically discrete units suited as unitary dosages for the subject to be treated, each unit containing a predetermined quantity of an active agent selected to produce the desired therapeutic effect, optionally together with a pharmaceutically acceptable carrier, which may be provided in a predetermined amount. The unit dosage form may be, for example, a volume of liquid (e.g., a pharmaceutically acceptable carrier) containing a predetermined quantity of a therapeutic agent, a predetermined amount of a therapeutic agent in solid form such as a tablet, caplet, capsule, or the like, an implant containing a predetermined amount of a therapeutic agent, a plurality of nanoparticles or microparticles that collectively contain a predetermined amount of a therapeutic agent, etc. It will be appreciated that a unit dosage form may contain a variety of components in addition to the therapeutic agent. For example, pharmaceutically acceptable carriers, diluents, stabilizers, buffers, preservatives, excipients, etc., may be included.

“Variant polynucleotide” with reference to any of the naturally occurring polypeptides mentioned herein means a polypeptide with a sequence having at least 80% amino acid sequence identity with the full-length native polypeptide sequence, preferably at least about 85% amino acid sequence identity, more preferably at least 90% amino acid sequence identity, more preferably at least 95% amino acid sequence identity, more preferably at least 99% amino acid sequence identity with a full-length native sequence.

“Vector” refers to a nucleic acid molecule capable of mediating entry of, e.g., transferring, transporting, etc., a second nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (typically DNA molecules although RNA plasmids are also known), cosmids, and viral vectors. As is well known in the art, the term viral vector may refer either to a nucleic acid molecule (e.g., a plasmid) that includes virus-derived nucleic acid elements that typically facilitate transfer or integration of the nucleic acid molecule (examples include retroviral or lentiviral vectors) or to a virus or viral particle that mediates nucleic acid transfer (examples include retroviruses or lentiviruses). As will be evident to one of ordinary skill in the art, viral vectors may include various viral components in addition to nucleic acid(s).

### III. Modulating Cognitive Function by Modulating Translation and MAP Kinase Pathways

As mentioned above, the inventors discovered that translational regulation is of key importance in the mechanisms underlying long-lasting forms of synaptic plasticity and memory. Specifically, as described in the Examples, the inventors showed that ERK-dependent signaling regulates protein synthesis in response to multiple forms of neuronal activity in hippocampal neurons and is needed for the establishment of long term memory. The inventors further showed that ERK induces translation of a broad range of neuronal RNAs and that ERK activation is required for specific phosphorylation of multiple components of the translation machinery in response to neuronal activity, providing a molecular mechanism for the observed dependence of translational induction on ERK activation. Thus the invention provides compositions and methods for modulating cognitive function by intervening in one or more pathways that regulate translation, e.g., a MAP kinase (MAPK) pathway.

In certain embodiments of the invention an agent that increases or enhances translation is administered to a subject in need thereof, e.g. a subject suffering from or at risk of a disease, disorder, or condition characterized by cognitive impairment or loss of cognitive function. The agent may, for example, activate one or more components of the translational machinery or inhibit a negative regulator of translation. The agent may increase expression or activity of one or more components of the MAP kinase signaling pathway, e.g., may directly or indirectly increase ERK or p38 MAPK activity. Average expression or activity of any one or more components may be increased by, e.g., at least 20%, 50%, 75%, 100%, 150%, 200%, 300%, 400%, 500%, or more, relative to its average value in the absence of the agent. The disease, disorder, or condition may be, e.g., benign senescent forgetfulness, age-associated memory impairment, age-associated cognitive decline, mild cognitive impairment, Alzheimer's disease, dementia due to any of a variety of causes, trauma-associated cognitive impairment, toxin-associated cognitive impairment, etc. The subject may be, e.g., at least about 50 years of age.

In certain embodiments of the invention an agent that decreases or inhibtis translation is administered to a subject in need thereof, e.g. a subject suffering from or at risk of a disease, disorder, or condition characterized by cognitive impairment or loss of cognitive function. The subject may be one in whom excessive protein synthesis and/or excessive translation occurs. Without wishing to be bound by any theory, excessive protein synthesis and/or translation may at least in part be responsible for certain diseases, disorders, or conditions of which cognitive impairment is often a feature or may play a role in the cognitive impairment that often occurs in these diseases, disorders, or conditions. “Excessive protein synthesis and/or translation” in this context means that the subject synthesizes an abnormally large amount of one or more proteins in one or more cell types, tissues, organs, organ systems, etc., and/or that one or more components of the translation machinery is abnormally activated or overexpressed and/or that one or more negative regulators of translation is abnormally inactivated, defective, or underexpressed. Alternately the subject may fail to appropriately degrade one or more protein(s), resulting in abnormally elevated levels of such protein(s). A determination of what constitutes “abnormal” will typically be made with respect to the average level of expression and/or activity and/or the range of expression level and/or activity observed in subjects having similar age, developmental status, etc. For example, a value that lies more than 2 standard deviations from an average value may be considered “abnormal”. In other embodiments a value that lies more than 3 standard deviations from an average value may be considered “abnormal”. The abnormal value may be, e.g., a protein synthesis rate, a steady state amount of protein, an average percent of a particular component of the translation machinery and/or MAPK or mTOR pathway that is phosphorylated, etc. The excessive protein synthesis may occur, e.g., in the CNS, e.g., in neurons, glial cells, or both. The agent may, for example, inhibit one or more components of the translational machinery or activate a negative regulator of translation. The agent may inhibit or decrease expression or activity of one or more components of the MAP kinase and/or mTOR pathway, e.g., may directly or indirectly decrease ERK, p38 MAPK, or mTOR activity. Average expression or activity of any one or more components may be decreased by, e.g., at least 20%, 50%, 75%, 100%, 150%, 200%, 300%, 400%, 500%, or more, relative to its average value in the absence of the agent.

The disease, disorder, or condition may be, e.g., mental retardation (due to any of a variety of causes), fragile X syndrome, tuberous sclerosis, or autism. The age of the subject may be, e.g., equal to or less than 3 months, 6 months, 1 year, 2 years, 3, years, 4 years, 5 years, or 10 years, or the age of the subject may fall within any two of these ages. Without wishing to be bound by any theory, the proposed functions of the gene products mutated in fragile X syndrome (FXS) and tuberous sclerosis complex (TSC), two forms of mental retardation that exhibit high phenotypic overlap with autism, have implicated defective translational control in the pathogenesis of mental retardation and autism. The fragile X mental retardation protein (FMRP) appears to function as a translational repressor, though its mechanism of action is not fully understood (Jin and Warren, 2003). The TSC proteins (TSC1, hamartin; TSC2, tuberin) act upstream of mTOR to repress its activity and inhibit downstream translational responses (see, e.g., Zhang et al, 2003 and Kwiatkowski and Manning, 2005, and references therein). In addition, upregulation of the ERK pathway and functional inactivation of the TSC1/2 complex through direct phosphorylation of TSC2 by ERK may play an important role in disease pathogenesis (see, e.g., Ma et al., 2005). Thus, loss of the normal controls on neuronal protein synthesis during brain development may represent a common pathway leading to mental retardation and autism.

A. MAP Kinase Pathways and Components Thereof

This section describes MAP kinase pathways and regulators of such pathways. In accordance with the invention, intervening in such pathways modulates translation and thus modulates cognitive function. The genes and proteins discussed herein are targets for intervention to modulate cognitive function and are targets in certain of the screening assays described below.

MAP kinases (mitogen-activated protein kinases) constitute a family of conserved serine/threonine kinases that are involved in transduction of external signals that regulate cell growth, division, differentiation, and apoptosis (Pearson, G., et al., Endocrine Reviews, 22(2): 153-183, 2001; Luttrell, D. and Lutrell, L., Assay and Drug Development Technologies, 1(2): 327-338, 2003). MAP kinases are activated by phosphorylation cascades in which two upstream protein kinases activated in series lead to activation of a MAP kinase. Additional kinases may act yet further upstream in the activation process. At least 6 MAP kinase pathways are known. Of primary interest herein is the pathway culminating in the activation of the MAP kinases ERK1 (extracellular signal-regulated kinase 1) and ERK2. ERK, as used herein, generally refers to ERK1 and ERK2 since these proteins are highly similar structurally and functionally, having an ˜85% amino acid identity overall, with much greater identity in the regions involved in substrate binding. Additional ERK isoforms (e.g., ERK3-6) are also known. Also of interest is the pathway culminating in activation of p38 MAP kinase.

For the currently known MAP kinase cascades, the kinase immediately upstream of the MAP kinase is a member of the MAP/ERK kinase (MEK or MKK) family. These are dual specificity kinases that phosphorylate specific serine/threonine and tyrosine residues in their particular MAP kinase substrates. In terms of their protein targets, MEKs have a narrow substrate specificity, phosphorylating only one or a small number of MAPKs. MEK1 and MEK2 are the MEKs which activate ERK1 and ERK2. MEKs themselves are activated by phosphorylation, which is mediated by MEK kinases (MEKKs), of which a large number are known. Of particular interest herein are the MEKKs that activate MEK1 and MEK2. These MEKKs include members of the Raf family of protein kinases, comprising A-Raf, B-Raf, and Raf-1. B-raf is most highly expressed in neural tissues and testis.

Further upstream of the MEKKs are a variety of other signal transduction elements. Rafs are regulated by members of the small G protein family such as various Ras proteins, Rap proteins, and multiple protein kinases including Src, protein kinase C (PKC), PAK, and Akt. Ras and Rap proteins are in turn regulated by guanine nucleotide exchange factors (GEFs), guanine nucleotide release factors (GRFs), and by phosphorylation by various kinases.

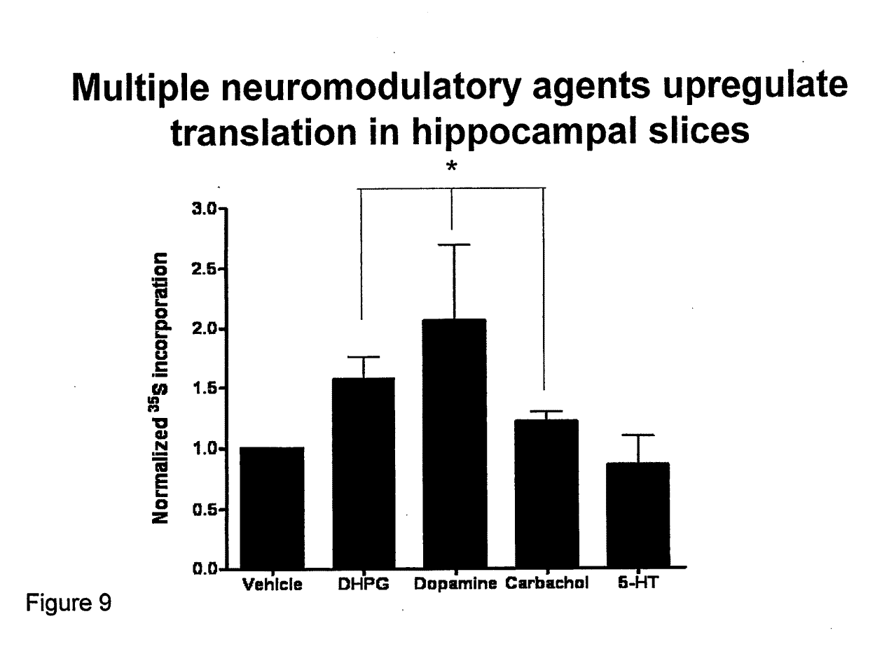

In many cases, activation of MAP kinases, including ERK1 and ERK2 results from stimulation of cell surface receptors which fall into two general classes: (i) receptor tyrosine kinases (RTKs); and (ii) G protein coupled receptors (GPCRs) (see FIG. 9).

Stimulation of RTKs increases kinase activity of the receptor, often leading to autophosphorylation, which results in formation of multiprotein complexes. These complexes in turn frequently activate small GTP proteins of the Ras superfamily. Complex formation involves recruitment of additional proteins via interaction of specific protein domains, e.g., SH2 and/or SH3 domains, leading to engagement of guanine nucleotide exchange factors that in turn stimulate activity of small G proteins such as Ras family members. As mentioned above, these small G proteins then activate MEKKs, leading ultimately to MAPK activation. Thus in accordance with the invention, modulation of MAPK activity may be achieved by activating or inhibiting RTKs, e.g., MAPK activity may be enhanced by activating one or more RTKs, or MAPK activity may be decreased by inhibiting one or more RTKs. Particular RTKs of interest herein include, but are not limited to, insulin receptor, epidermal growth factor receptor, fibroblast growth factor receptor, platelet-derived growth factor receptor, insulin-like growth factor receptors, and neurotrophin receptors.

Signaling to MAPKs via GPCRs also involves regulation of small G proteins via a variety of pathways. GPCRs are coupled to G proteins of 3 general classes, i.e., Gs, Gi, and Gq, and members of each class can modulate MAPK activity via a diverse set of mechanisms involving the Gα and/or Gβγ subunits, which interact with various kinases and phospholipases. In particular, stimulation of many G proteins activates or inhibits adenylyl cyclase, which generates cyclic AMP (cAMP). A number of isoforms of adenylyl cyclase are known. cAMP in turn activates protein kinase A (PKA), which activates the small G protein Rap1, which in turn activates the MEKK B-Raf. Many G proteins utilize the inositol 1,4,5 trisphosphate (IP3)/diacylglycerol (DAG) signal transduction pathway. Agonist binding to the GPCR leads to coupling with a G protein, e.g., Gq, which leads to release of G-protein bound GDP, exchange for GTP, and dissociation of the G protein into α and βγ subunits, both of which activate various phospholipase C β (PLCβ) isoforms. Stimulation of PLCβ leads to hydrolysis of phosphatidylinositol species, resulting in formation of IP3 and DAG. IP3 then binds to a receptor on endoplasmic reticulum membranes, resulting in release of intracellular Ca⁺⁺ stores, which leads to activation of protein kinase C (PKC). PKC activation has a direct or indirect stimulatory effect on small G proteins that activate MEKKs. Thus in accordance with the invention, modulation of GPCR activity modulates MAPK activity, which in turn modulates translation and cognitive function. For example, MAPK activity may be enhanced by activating one or more GPCRs, or MAPK activity may be decreased by inhibiting one or more GPCRs. Particular GPCRs of interest herein include, but are not limited to, metabotropic glutamate receptors mGluR1 and mGluR5, dopamine receptors D1, D2, and D5, GABA receptors (e.g., GABA_{A} and GABA_{B} receptors), and β-adrenergic receptors (e.g., β1 and β2 receptors).

As discussed above, phosphorylation plays a major role in regulating the activity of many members of MAPK pathways. It will be understood that proteins whose activity is regulated by phosphorylation will also be regulated by dephosphorylation by one or more phosphatases. For example, if a protein is activated by phosphorylation of one or more residues it will typically be inactivated by dephosphorylation of one or more of these residue(s). If a protein is inhibited by phosphorylation of one or more residues it will typically be released from inhibition, or activated, by dephosphorylation of one or more of these residue(s).

B. Translation Pathways and Components Thereof

This section describes translation pathways involving the general translation machinery and regulators of such pathways. In accordance with the invention, intervening in such pathways modulates cognitive function. Certain of the components of the general translation machinery are preferred target for intervention. The proteins discussed herein are among the preferred targets in the screening assays described below.

Eukaryotic protein synthesis is conventionally divided into three steps: initiation, elongation, and termination. Initiation is the process in which a translation-competent ribosome is assembled at the start codon (e.g., AUG) on an mRNA. Elongation refers to the codon-dependent assembly of a polypeptide by sequential incorporation of amino acids. Termination refers to release of the polypeptide when the ribosome arrives at a stop codon. A variety of protein factors are involved at each of these steps. These proteins are referred to as eukaryotic initiation factors (eIF), elongation factors (eEF), and release factors (eRF). Initiation itself can be divided into a number of steps involving binding of the initiator methionyl-transfer RNA to the small (40S) ribosomal subunit to form the 43S pre-initiation complex, binding of the 43S complex to an mRNA to form a 48S pre-initiation complex, and binding of the large (60S) ribosomal subunit to the 48S complex after the latter has scanned the mRNA to identify the start codon, thus forming the 80S ribosome. Most translational regulation occurs during the initiation steps.

The identities and roles of various components of the general translation machinery are well understood. The following short descriptions are provided to facilitate understanding of the invention. The reader is directed to Hershey, J W, et al., (eds), Translational Control, Cold Spring Harbor Laboratory Press, Cold Spring Harbor, 1996; and Sonenberg, N, et al. (eds.) Translational Control of Gene Expression, Cold Spring Harbor Laboratory Press, Cold Spring Harbor, 2000; and Klann, E. and Dever, T E, Nature Reviews Neuroscience, 5:931-942, 2004, for more detailed information.

Briefly, protein components of the general translation machinery include the following initiation factors: (1) eIF1A; (2) eIF2, which consists of a regulatory subunit (eIF2α) and a guanine nucleotide exchange factor (eIF2β) for the regulatory subunit; (3) eIF3; (4) eIF4A; (5) eIF4B; (6) eIF4E; (7) eIF4F, which consists of eIF4A, eIF4E, and eIF4G; (8) eIF4G; (9) eIF5; and (10) eIF5B. Many of these components are targets for regulation by a variety of translational regulators, including a number of different kinases. An additional component of the general translation machinery that is a target for regulation is the ribosomal protein S6. S6 is phosphorylated by an upstream S6 kinase, frequently referred to as p70 S6 kinase, which may be a more important inducer of translation. S6 may be a marker for the potentially more relevant S6 kinase activity, with the S6 kinase having other critical substrates.

As mentioned above, a number of proteins regulate one or more steps in initiation and/or other steps in translation. Such regulators include (1) 4E-BP, which binds to eIF4E and inhibits formation of eIF4F, thereby inhibiting initiation; (2) CPEB, an RNA-binding protein that recognizes the CPE in the 3′ UTR of target mRNAs; and (3) Maskin, a protein that binds to CPEB and eIF4E and blocks initiation of a target mRNA by preventing eIF4F formation. The activity of many initiation factors (and also factors involved in other protein synthesis steps) is governed at least in part by their phosphorylation state, which is in turn controlled by a variety of kinases and phosphatases. Kinases that regulate translation include protein kinase-RNA regulated (PKR), haem-regulated initiation factor 2α kinase (HR1), general control non-derepressible 2 kinase (GCN2), eIF2α kinase 3 (PERK), glycogen synthase kinase 313, ERK, phosphoinositide 3-kinase (PI3K), mammalian target of rapamycin (mTOR), p38 mitogen-activated protein kinase (p38MAPK), MAPK-interacting serine/threonine kinases 1 and 2 (Mnk1 and Mnk2), phosphoinositide-dependent kinase 1 (PDK1), Aurora family kinases, and p70 S6 kinases (e.g., S6K1, S6K2, etc).

Of particular interest herein is the regulation of eIF4E. eIF4E is subject to regulation by binding proteins known as 4E-BPs (4E-BP1, 4E-BP2, and 4E-BP3). When not bound to 4E-BP, eIF4E associates with eIF4G to form the eIF4F complex and thus promote initiation. 4E-BPs compete with eIF4G for binding to eIF4E and thereby block eIF4F formation. Binding of 4E-BPs to eIF4E is regulated by phosphorylation. Hypophosphorylated 4E-BPs bind to eIF4E and thus inhibit translation, while multiply phosphorylated 4E-BPs cannot bind to eIF4E, thus allowing formation of eIF4F. 4E-BPs are phosphorylated by ERK, pI3K, and mTOR. eIF4E is also regulated directly by phosphorylation. In particular, Mnk1 and Mnk2 phosphorylate eIF4E, which leads to increased translation rates. Mnk1 and Mnk2 are phosphorylated and activated by the MAP kinases ERK and p38. Activators of the expression or activity of ERK, pI3K, mTOR, p38, Mnk1, and/or Mnk2 are of use in the present invention to activate or enhance translation. In addition, activators of the expression or activity any of the proteins known in the art that activate expression or activity of ERK, pI3K, mTOR, p38, Mnk1, and/or Mnk2 are of use in the present invention to activate or enhance translation. Inhibitors of the expression or activity of any of the proteins known in the art that inhibit expression or activity of ERK, pI3K, mTOR, p38, Mnk1, and/or Mnk2 are of use in the present invention to activate or enhance translation Inhibitors of the expression or activity of ERK, pI3K, mTOR, p38, Mnk1, and/or Mnk2 are of use in the present invention to repress or inhibit translation. In addition, inhibitors of expression or activity of any of the proteins known in the art that activate expression or activity of ERK, pI3K, mTOR, p38, Mnk1, and/or Mnk2 are of use in the present invention to repress or inhibit translation. Activators of the expression or activity of any of the proteins known in the art that inhibit expression or activity of ERK, pI3K, mTOR, p38, Mnk1, and/or Mnk2 are of use in the present invention to repress or inhibit translation.

As mentioned above, the inventors showed that translational regulation is of key importance during L-LTP and memory formation. The inventors further showed that 4E-BP phosphorylation is specifically regulated by ERK during hippocampal plasticity and memory. In particular, ERK inhibition blocked phosphorylation of eIF4E and 4EBP1 in hippocampal neurons and in hippocampal slices, whereas such phosphorylation occurred in the absence of ERK inhibition in response to L-LTP generating stimulation. In addition, ERK inhibition blocked phosphorylation of eIF4E under these conditions. Thus the inventors demonstrated that ERK plays a key role in regulating the phosphorylation state and thus activity of eIF4E via at least two distinct mechanisms during L-LTP and memory formation. Therefore, in accordance with the invention, modulation of the activity and/or abundance of ERK pathway components in turn modulates cognitive function. Given the role of other pathways, e.g., the mTOR pathway, in regulating certain of the same components of the general translation machinery as those regulated by ERK pathways, as well as in regulating other components, the invention also encompasses modulating components of the mTOR pathway (e.g., mTOR itself, Raptor, PI3 kinase, PTEN phosphatase, PKD1/2, Aid, MNK1/2, mTOR, PKCδ, Raptor, S6K1, S6K2, PP2A, tuberous sclerosis complex 1 (TSC1), and TSC2) (Hay, N. and Sonenberg, N., Genes & Dev., 18:1926-1945, 2004). For example, Raptor activates mTOR, while rapamycin and certain derivatives and analogs thereof inhibit mTOR. Thus the invention encompasses use of rapamycin or a derivative or analog thereof (e.g., temsirolimus (CCI-779), everolimus (RAD001; Certican), and AP23573) to inhibit mTOR, which would in turn inhibit translation. Rapamycin is a macrocyclic triene antibiotic produced by Streptomyces hygroscopicus which was found to have antifungal activity and has since been shown to be useful in treatment of a variety of diseases and conditions. See, e.g., U.S. Pat. Nos. 3,929,992; 3,993,749; U.S. Pub. No. 20010010920; WO 03/064383, WO 2004/026280, and US Pat. No. 6,384,046 and references cited in each of the foregoing for additional information about rapamycin and certain analogs and derivatives thereof.

While the above discussion has focused on initiation, it is evident that components of the general translation machinery involved in elongation and termination are also targets for regulation. One such component is eEF2, which is regulated by phosphorylation. In particular, phosphorylation by the eEF2 kinase inhibits eEF2 activity and reduces peptide elongation. eEF2 kinase is in turn regulated by activation of mTOR, which results in phosphorylation of the kinase and decreases its activity.

IV. Agents Useful for Modulating Cognitive Function

A variety of compounds that interact with the targets and/or modulate the pathways described above are known and may be used in the practice of the methods. This section describes certain exemplary agents. These agents fall into a number of different chemical classes. The use of certain compounds within these classes may have been previously proposed for modulating cognitive function and/or for other purposes. Accordingly, the invention includes embodiments in which any specific subset of such compounds (e.g., subsets in which one or more of the compounds falling into the compound class) is excluded. These individual subsets are not explicitly set forth herein, since it would be unduly burdensome to do so. Furthermore, the classification of any of the agents described herein into a specific class is not intended to imply that the agent does not also fall into one or more of the other classes. However, the invention provides new methods for using compounds that have been previously proposed for modulating cognitive function and provides new compositions and formulations of such compounds.

In certain embodiments of the invention a compound is administered in an amount sufficient to activate or inhibit the MAPK signaling pathway or one or more components thereof. In certain embodiments of the invention the compound is administered in an amount sufficient to activate ERK or p38 MAPK.

A. Modulators of Tyrosine Kinase Receptors

Exemplary agents that modulate tyrosine kinase receptors include a variety of agonists including, but not limited to, insulin, neurotrophins, growth factors such as EGF, etc. In certain embodiments of the invention agonists of tyrosine kinase receptors are useful to enhance cognitive function while antagonists are useful to inhibit cognitive function. For example, in certain embodiments of the invention agonists of tyrosine kinase receptors enhance MAPK activity, thereby enhances translation and enhances cognitive function, e.g., in subjects suffering from or at risk of Alzheimer's disease, age-associated memory impairment, mild cognitive impairment, trauma-associated cognitive impairment, toxin-associated cognitive impairment, or dementia. In certain embodiments of the invention antagonists of tyrosine kinase receptors inhibit or reduce MAPK activity, which enhances translation and enhances cognitive function, e.g., in subjects suffering from or at risk of mental retardation, fragile X syndrome, tuberous sclerosis, or autism.

B. Modulators of G Protein Coupled Receptors

Exemplary GPCR agonists that activate MAPK include a variety of peptides such as bombesin, bradykinin, endothelin-1, somatostatin, IL-8, LHRH, C5a, TRH, fMLP, oxytocin, angiotensin, thrombin, and integrin ligands. Lipid activators include thromboxane A2, prostaglandin F2, platelet-activating factor (PAF), sphingosine-1-phosphate, and lysophosphatidic acid. Neurotransmitter activators include dopamine (activates dopamine receptors), noradrenaline (activates adrenergic receptors), serotonin, glutamate (activates metabotropic and ionotropic glutamate receptors), acetylcholine, and adenosine.

It is noted that numerous pharmaceutical agents that act on GPCRs of various types are known and that screens for small molecule activators and inhibitors of GPCRs can readily be performed to identify agents acting on additional GPCRs without undue experimentation. In certain embodiments of the invention agonists of GPCRs that activate MAPK are useful to enhance cognitive function while antagonists of such GPCRs are useful to inhibit cogntive function. For example, numerous dopamine receptor agonists or antagonists are known in the art. The D1 agonist SKF-38393 and the D2 agonist quinpirole are exemplary dopamine receptor agonists.

C. Modulators of Beta-Adrenergic Receptors

A large number of molecules that modulate β-adrenergic receptors are known in the art. An exemplary activator is isoproteronol. An exemplary inhibitor is propranolol. See, e.g., Goodman & Gilman, supra for information regarding additional agents including agents selective for β1 or β2 receptors.

D. Modulators of Metabotropic Glutamate Receptors mGluR1 and/or mGluR5

DHPG (3,4-Dihydroxyphenylglycol) is used herein as an exemplary agonist of mGluR1. [(3S,4S)-DHGA] is another such agonist. Additional molecules that modulate mGluR1 and/or mGluR5 activity are known in the art. See, e.g., U.S. Pat. No. 6,706,707 and US Pub. No. 20040132792.

E. Modulators of NMDA Receptors

A large number of molecules that modulate NMDA receptor activity are known in the art. See, e.g., U.S. Pat. Nos. 6,635,270; 5,804,550; 5,807,859 and US Pub. No. 20020035145.

F. Modulators of GABA Receptors

A large number of molecules that modulate GABA receptor activity are known in the art. See, e.g., U.S. Pat. Nos. 6,833,385; 6,828,322; 6,723,735; 6,211,365; 6,127,418, etc.

G. Modulators of Receptor Tyrosine Kinases that Activate MAPK and/or mTOR

Exemplary agonists of RTKs that activate MAPK include EGF (EGFR, multiple subtypes), FGF (activates FGFR, multiple subtypes), PDGF (activates PDGFR), NGF (trkA), BDNF (activates trkB), activates NT-3 (trkC), activates insulin receptor (IR), IGF-1 (activates insulin-like growth factor receptor (IGFR).

H. Modulators of Adenylyl Cyclase

Activators of adenylyl cyclase include forskolin, Sp-cAMPS and analogs.

I. Modulators of Phosphodiesterase

Inhibitors of phosphodiesterase include, but are not limited to, rolipram. A large number of additional molecules that activate or inhibit phosphodiesterase are known in the art. See, e.g., U.S. Pat. Nos. 6,316,457; 6,136,810; and 6,080,782.

J. Modulators of mTOR Pathway Components

A number of the GPCR agonists mentioned above also activate the mTOR pathway.

K. Modulators of MAPK

Phorbol esters are exemplary activators of MAPK.

L. Modulators of Negative Regulators of Translation

As described above, a number of components, e.g., 4E-BP and PTEN, act as negative regulators of translation. These can be specifically inhibited using RNAi agents targeted to a transcript that encodes the particular regulator of interest.

M. Modulators of Muscarinic Acetylcholine Receptors

Agonists and antagonists of muscarinic acetylcholine receptors are known in the art and are of use in this invention. Examples of muscarinic agonists include carbachol, pilocarpine, bethanechol, and methacholine. Oxotremorine and McNA343 are M1-selective agonists. Muscarinic antagonists include atropine, scopolamine, glycopyrrolate, and ipratropium. Other agents with anticholinergic activity include tricyclic antidepressants and certain anti-histamines. In certain embodiments of the invention administration of a muscarinic agonist enhances translation, which results in enhancement of cognitive function, e.g., in a subject suffering from or at risk of Alzheimer's disease, age-associated memory impairment, mild cognitive impairment, trauma-associated cognitive impairment, toxin-associated cognitive impairment, etc. In certain embodiments of the invention administration of a muscarinic antagonist reduces translation, which results in enhancement of cognitive function, e.g., in a subject suffering from or at risk of mental retardation, fragile X syndrome, tuberous scleroris, or autism.

### V. Screening Methods and Related Reagents

### A. General Considerations

The invention provides assays that can be used to screen for agents that modulate cognitive function. The assays include both cell-free and cell-based assays. Preferably the assays are performed in a high throughput format. For example, preferably the assays are performed in microtiter plates, e.g., 96-well, 384-well, 1536-well, 3456-well plates and utilize microtiter-plate based liquid handling devices, endpoint plate readers, and/or microtiter-plate based robotic systems, etc. The assays optionally include steps of (i) contacting a hippocampal slice or neuronal culture with a candidate agent and measuring L-LTP and/or (ii) administering a candidate agent to a non-human animal (e.g., a rodent or non-human primate) and performing a behavioral test that correlates with memory and/or cognitive function in a human being.

Targets of the various assays include, but are not limited to, (i) proteins that are components of the general translation machinery; (ii) proteins that regulate the activity or abundance of one or more components of the general translation machinery; and (iii) proteins that are components of a MAPK pathway. Many of these proteins fall into the following classes: receptor tyrosine kinases, G protein coupled receptors, non-receptor kinases, phosphatases, G proteins, small GTP proteins, GTPase activating proteins, guanine nucleotide exchange factors, guanine nucleotide release factors, etc. Methods for identifying agents that activate or inhibit proteins in these various classes are generally known in the art and may be employed to identify agents that activate or inhibit particular members of these classes discussed in Section III. Certain suitable assays are described below, but the screening methods are not limited to these. Generally, the assays are used to identify agents that (i) modulate an activity (e.g, an enzymatic activity) of a target; (ii) alter the synthesis, degradation, or half-life of a target; (iii) alter an interaction of a target with one or more other targets, substrates, or other proteins, polynucleotides, lipids, carbohydrates, or other molecules in a biological system; (iv) alter the subcellular localization of a target, etc.

In general, the assays will make use of a biological system, which may be a cell-free or cell based system. Cell-free assays refer to assays that are performed using a biological system that does not include intact cells but includes one or more biological macromolecules, e.g., protein(s) and/or nucleic acid(s). Cell-based assays employ intact cells. The cells may be maintained in culture. They may be present in a tissue slice, in which the natural architecture of the tissue is maintained largely as found in an intact organism. The cells may, but need not be, neuronal cells, e.g., hippocampal neurons. The cells may be primary cells, which may be used directly following removal from an organism. The cells may be passaged one or more times prior to use. Cells of an immortalized cell line, e.g., a neuronal cell line such as PC-12 cells, may be used. Preferably the cells are mammalian cells.

The biological system may comprise purified or partially purified nucleic acids and/or proteins, cell lysates, cellular fractions such as cell membrane preparations, cells, tissue slices, animals, etc. The biological macromolecules may be chemically synthesized, produced by recombinant DNA technology, obtained from natural sources (e.g., from cells such as those mentioned below, from organisms), etc.

Briefly, a “recombinant protein” refers to a protein which is produced by recombinant DNA techniques, wherein generally, DNA encoding a polypeptide is inserted into a suitable expression vector which is in turn introduced into a host cell to produce the recombinant protein. Vectors may be introduced into cells using any of a variety of suitable methods. Methods for introducing nucleic acids into cells are well known in the art. One of ordinary skill will be able to select appropriate cells for expression and an appropriate method (e.g., calcium phosphate or lipid-mediated transfection, electroporation, bacterial or fungal transformation, etc.) for introducing a nucleic acid into the cells, taking into consideration the cell type, etc. Suitable host cells for producing recombinant proteins include bacteria, yeast, insect cells, mammalian cells such as COS cells, CHO cells, HeLa cells, NIH3T3 cells, etc. Certain of these cells maybe used in the cell-based assays described below. The biological macromolecules may be any of the components of the general translation machinery or regulators thereof mentioned above and/or may be any of the components of a MAPK pathway mentioned above. In most cases the components of interest are proteins, although nucleic acids are not excluded. Preferred biological macromolecules for use in the assays include (i) proteins that are components of the general translation machinery; (ii) proteins that regulate the activity or abundance of one or more components of the general translation machinery; (iii) proteins that are components of a MAPK pathway. The biological macromolecules may be variants of any of the foregoing proteins. Certain variants are specifically engineered, e.g., by point mutation, deletion, truncation, etc., to alter a biological activity of a protein. For example, constitutively active forms can be produced. In active forms can be produced, which may act antagonistically to a naturally occurring protein, e.g., by competing for binding to another component in a pathway. In certain embodiments an active fragment of a protein is used. Typically the active fragment comprises at least 50 amino acids of the complete protein. Certain fragments retain at least one biological activity of the complete protein, e.g., enzymatic activity, inhibitory activity, binding activity, etc. Additional preferred biological macromolecules include substrates for any of the foregoing proteins. For example, myelin basic protein and microtubule-associated protein 2 are substrates for MAP kinase. The biological macromolecules may be modified for use in the assays. For example, a protein may incorporate a heterologous sequence, e.g., an epitope tag such as a GST, Myc, HA, FLAG™, maltose-binding domain, 6×-His or other metal binding moiety, etc. The biological system may comprise one or more antibodies, agonists, antagonists, etc., for any of the afore-mentioned proteins, inhibitors of transcription, inhibitors of translation, etc.

In certain embodiments human biological macromolecules are used. It may be desirable to perform assays using protein isoforms that are expressed in the brain. The isoforms may be CNS-specific, e.g., primarily or exclusively expressed in one or more brain regions, or they may also be expressed in one or more other organs. In certain embodiments an isoform whose average expression in the brain, or in a region thereof such as the hippocampus, cortex, amygdala, etc., is at least 2-fold, at least 4-fold, or at least 10-fold greater than its average expression in one or more other tissues selected from the group consisting of: liver, lung, heart, spleen, pancreas, kidney, skeletal muscle, testis, ovary, thyroid, fat, or skin, is used.

In general, the screening methods are used to identify activators of translation and/or activators of a MAPK pathway for purposes of enhancing cognitive function. It will be appreciated that inhibitors of negative regulators of these pathways will have an activating effect. Conversely, the screening methods are used to identify inhibitors of translation and/or inhibitors of a MAPK pathway for purposes of reducing cognitive function. It will be appreciated that activators of negative regulators of these pathways will have an inhibitory effect on a pathway. Thus both activators and inhibitors are of interest, depending on whether the particular target is a positively or negatively acting component of a pathway. In any of the assays described herein, the effect of a candidate compound on a biological system may be compared with a control system lacking the compound or containing less of the compound. Preferably compounds that cause a statistically significant effect are identified as candidate modulators of cognitive function.

The various proteins of interest herein are referred to by their common names as understood by one of ordinary skill in the art. Sequence information is readily available for each of these proteins, e.g., in public databases such as GenBank. One of ordinary skill in the art will be able to identify the appropriate protein and corresponding nucleic acid sequences for any particular species of interest using the relevant scientific literature and databases. It is noted that frequently a number of entries for each protein appear. Such entries are collected under a specific GeneID in GenBank. As known to one of ordinary skill in the art, the website for finding GeneIDs is Pubmed, just as for finding GenBank accession numbers. The website has URL www.pubmed.com. The GeneID search is performed by selecting “Gene” from the pull-down menu at the top left (below “nucleotide”, “protein”, etc.). The following list provides GeneIDs for the human forms of a number of the components mentioned herein.

ERK1=GeneID 5595

ERK2=5594

MEK1=5604

MEK2=5605

Raf-1=5894

A-Raf=369

B-Raf=673

Ha-Ras=3265

K-Ras=3845

N-Ras=4893

Rap1 (isoforms A and B): 5906, 5908

eIF4E=1977

4E-BP1=1978

4E-BP2=1979

4E-BP3=8637

Mnk1=8569

Mnk2=2872

S6=6194

p70 S6K1=6198

p70 S6K2=6199

mTOR=2475

Akt/PKB, 3 isoforms (Akt1-3): 207, 208, 10000

PDK1=5170

PTEN=5728

TSC1=7248

TSC2=7249

Multiple isoforms (at least 8) of adenylyl cyclase.

### B. Translation Assays

The invention provides a method of identifying an agent that modulates cognitive function comprising: (a) providing a biological system for detecting an increase or decrease in translation; (b) contacting the system with a candidate agent; (c) determining whether the agent increases or decreases translation; and (d) identifying the agent as a candidate modulator of cognitive function if translation is increased or decreased.

The biological system preferably contains components of the general translation machinery described above, amino acids, ribosomes, GTP, and mRNA molecules. The biological system may contain one or more translational regulators. In certain embodiments the biological system contains one or more MAPK pathway component(s). In general, any cell lysate can be used. Known in vitro translation systems include, e.g., reticulocyte lysate systems, wheat germ extract systems, etc. In certain embodiments a commercially available in vitro translation system is used. Alternately, purified or partially purified components can be combined in a suitable vessel. Translation can also be measured in cultured cells, tissue slices, specific organs, or whole animals.

Translation may be measured using a variety of methods. For example, measuring incorporation of a radioactive moiety, e.g., ³⁵S into proteins is one common approach. Certain of the assays involve a translational reporter construct that is used to measure translation and to determine the effect of a candidate agent on translation. In general, such a construct comprises an mRNA that encodes a readily detectable moiety. Translation of the RNA results in production of the readily detectable moiety. Measuring the amount or activity of the readily detectable moiety provides an indication of the level of translation of the reporter construct. In general, expression of a readily detectable marker within a cell results in the production of a signal that can be conveniently detected and/or measured. The process of detection or measurement may involve the use of additional reagents and may involve one or more processing steps. For example, where the detectable marker is an enzyme, detection or measurement of the marker will typically involve providing a substrate for the enzyme. Preferably the signal is light, fluorescence, luminescence, bioluminescence, chemiluminescence, enzymatic reaction products, or color. Thus preferred readily detectable markers include fluorescent proteins such as green fluorescent protein (GFP) and variants thereof. A number of enhanced versions of GFP (eGFP) have been derived by making alterations such as conservative substitutions in the GFP coding sequence. Other readily detectable markers that produce a fluorescent signal include red, blue, yellow, cyan, and sapphire fluorescent proteins, reef coral fluorescent protein, etc. A wide variety of such markers is available commercially, e.g., from BD Biosciences (Clontech). Enzymatic markers include, e.g., β-galactosidase, chloramphenical acetyltransfersase, alkaline phosphatase, horseradish peroxidase, etc. Additional readily detectable markers preferred in certain embodiments of the invention include luciferase derived from the firefly (Photinus pyralis) or the sea pansy (Renilla reniformis). Other reporters include enzymes that cleave a substrate, wherein the substrate has a fluorescent moiety and a fluorescence quencher attached thereto. Cleavage separates the flourescent moiety from the quencher, resulting in a detectable increase in the fluorescent signal.

The reporter construct may comprise one or more translational regulatory sequences. However, for purposes of ascertaining whether an agent modulates activity of a component of the general translation machinery, preferably the reporter does not contain such sequences. In certain embodiments the reporter does not contain a CPE, a hexamer sequence, or both. In certain embodiments the reporter comprises a polyA tail. Preferably the polyA tail is a minimal polyA tail, e.g., a tail that is approximately 20 nucleotides in length. In other embodiments a longer polyA tail, e.g., approximately 20-50 nucleotides, approximately 50-100 nucleotides, greater than 100 nucleotides, etc., is used. An exemplary reporter construct is described in Example 4.

In certain embodiments of the invention a selection is used rather than a screen. In these embodiments translation of the reporter confers a growth advantage on cells or is necessary for their survival. For example, the reporter construct may encode an essential gene in a cell lacking a functional version of the gene. The reporter construct may encode a drug resistance marker, e.g., an enzyme that metabolizes a toxic compound or pumps it out of the cell. Candidate agents are applied to populations of cells, which are maintained in culture for a period of time. If the cells survive or grow robustly, the agent is identified as an a candidate enhancer of translation.

Alternately, phenotypic changes caused by expression of a reporter construct (or an endogenous protein) can be utilized as a readout for a translation assay. For example, overexpression of certain proteins is known to lead to increases in cell proliferation, changes in cell shape, etc. Such a protein may be expressed in a cell, and the ability of an agent to increase or decrease translation is assessed by observing a change in the phenotype associated with expression of the protein.

### C. Phosphorylation Assays

Phosphorylation of MAP kinases and of the upstream MEKs and MEKKs is required for their activity. These activated kinases in turn phosphorylate a variety of substrates. For example, the inventors showed that activation of ERK1 and ERK2 during hippocampal L-LTP and memory formation induced phosphorylation of components and regulators of the general translation machinery including eIF4E and 4E-BP. S6 was also phosphorylated. Analysis of the phosphorylation state of various components of the general translation machinery can thus be used as a readout of the activity of MAP kinase pathways. Methods for detecting and measuring the phosphorylation of proteins of interest such as MAP kinases and their regulators and substrates are known in the art. In one approach, cells are cultured in the presence of radiolabelled phosphate, and the protein of interest is isolated or detected (e.g., using antibodies). The amount of radiolabel incorporated into the protein provides a measure of the extent of its phosphorylation.

Nonradioactive systems are also available. For example, IMAP® technology (Molecular Devices) is based on the high affinity binding of phosphate at high salt concentration by immobilized metal (MIII) coordination complexes on nanoparticles. This IMAP “binding reagent” complexes with phosphate groups on phosphopeptides generated in a kinase reaction. Such binding causes a change in the rate of the molecular motion of the peptide, and results in an increase in the fluorescence polarization value observed for the fluorescein label attached at the end of the peptide. This assay is applicable to a wide variety of kinases without regard to the substrate peptide sequences.

Another approach involves the use of antibodies that are specific for proteins having a particular phosphorylation state. A number of such antibodies are available. As described in the Examples, the inventors used antibodies specific for dually-phosphorylated ERK1/2, phospho-S6 (S235/S236), phospho-eIF4E (S209), and phospho-4E-BP to assess the phosphorylation state of these components. More generally, antibodies against phosphothreonine, phosphoserine, and phosphotyrosine are available.

### D. Assays for RTK Agonists and Antagonists

A variety of methods are used in the art to screen for activators and/or inhibitors of RTKs. Suitable assays are described, for example, in Rahman, A., et al., Bioassay Techniques for Drug Development, Harwood Academic Publishers, Amsterdam, 2001. These include measurements of the phosphorylation state of particular substrates, e.g., those mentioned above. Such measurement may make use of antibodies that specifically recognize substrates having different phosphorylation states. Activation of many RTKs results in transcriptional activation. Therefore, transcriptional reporter assays can be used. For example, reporter constructs containing a suitable transcription factor response element operably linked to a coding sequence for a readily detectable marker is introduced into cells. The cells may be transiently or stably transfected with the reporter construct. Activation of the RTK increases transcription, resulting in a measurable change in signal. Several RTKs stimulate PLC isoforms, resulting in increased IP3, DAG, and/or Ca⁺⁺, which can be measured as described in the following section.

Stimulation of RTKs frequently induces cell proliferation. Thus agents that activate RTKs may be identified by using cell proliferation as a readout. Antagonists of RTKs may be identified by applying a known agonist and screening for agents that reduce the extent of proliferation induced by the known agonist.

Yet another method for identifying agonists and antagonists of RTKs and/or GPCRs is a melanophore functional receptor assay (Bunsen Rush, Inc.). This assay technology utilizes the natural response of pigment-bearing frog melanocytes, which undergo rapid optical density changes mediated by GPCRs and RTKs. Standard plate readers can be used to detect the change. Mammalian receptors are transfected into frog melanocytes, and application of ligands that activate the transfected receptors cause either aggregation or dispersion, depending on the particular signal transduction pathway of the receptor.

### E. Assays for GPCR Agonists and Antagonists

Detection of changes in the level of the various molecules downstream of activation of GPCRs (e.g., cAMP, DAG, IP3, Ca⁺⁺) forms the basis of a number of assays to identify agonists and antagonists of GPCRs. A number of kits for performing such assays are commercially available. Suitable assays are described, for example, in Rahman, A., et al., Bioassay Techniques for Drug Development, Harwood Academic Publishers, Amsterdam, 2001. Very briefly, such assays may involve cAMP response element reporter genes or measurement of cAMP, e.g., using scintillation proximity assays, ELISA assays, etc. A number of these are competitive assays that quantitate the binding of trace amounts of radiolabelled cAMP in the presence of unlabelled cAMP generated in the assay system. Kits for measuring IP3 and DAG are commercially available. More convenient assays, amenable to high throughput methods involve measurement of Ca⁺⁺ using calcium-sensitive dyes such as Fura-2 AM, Calcium Green AM, Fluor-3 AM, etc. Since IP3 causes release of intracellular Ca⁺⁺ stores, testing for agents that increase intracellular Ca⁺⁺ identifies agents that activate GPCRs coupled to PLCβ enzymes.

### F. GTPase Assays

Stimulation of both GPCRs and small G proteins can be measured by detecting GTPase activity. GTPase activity can be determined by a number of methods including measuring determining the breakdown of radiolabelled GTP using techniques that are known in the art. Methods for measuring activities such as guanine nucleotide exchange activity are also known in the art.

### G. Binding Assays

Binding assays are of use to identify agents that bind directly to target molecules such as components of the general translation machinery, regulators of translation, MAPK pathway components, etc.

Screening for agonists and antagonists of proteins, particularly receptors, has traditionally been performed using a variety of in vitro techniques such as radioligand binding assays, photocrosslinking, and affinity chromatography. These assays are based on the principles of a competitive binding assay in which a radiolabeled ligand competes with an unlabeled ligand for binding to a target protein, e.g., a receptor in a cell or an a cell membrane. The ability of a test agent to compete with a known ligand is assessed by measuring the binding of the labeled known ligand in the presence of a test agent. A decrease in the binding of the labeled known ligand indicates that the test agent binds to the target molecule. Whether the test agent acts as an agonist or antagonist is then determined using other methods. When performing such assays, any of the known ligands mentioned above for the various target proteins can be used. Methods for performing radioligand binding assays are well known in the art, and a number of kits are commercially available. See, e.g., Rahman, A., et al., Bioassay Techniques for Drug Development, supra. Examples of commercially available kits include the FlashPlate™ system (DuPont-NEN), the “Scintillation Proximity™” assay (Amersham), ScintiStrip™ plates (Wallac), etc. Ligands labeled with nonradioactive detectable moieties, e.g., fluorescent moities, may alternatively be used.

Methods based on fluorescence polarization and surface plasmon resonance are increasingly employed to detect molecular interactions. The phenomenon of surface plasmon resonance is used in Biacore systems (available from Biacore International AB, Neuchatel, Switzerland). Such systems can be used to detect interactions between a protein of interest and a test agent. As described by Biacore, detection using surface plasmon resonance sensors works as follows: As molecules are immobilized on a sensor surface the refractive index at the interface between the surface and a solution flowing over the surface changes, altering the angle at which reduced-intensity polarized light is reflected from a supporting glass plane. The change in angle, caused by binding or dissociation of molecules from the sensor surface, is proportional to the mass of bound material and is recorded in a sensorgram. When sample is passed over the sensor surface, the sensorgram shows an increasing response as molecules interact. The response remains constant if the interaction reaches equilibrium. When sample is replaced by buffer, the response decreases as the interaction partners dissociate.

As discussed above, a number of components of the general translation machinery interact with each other and/or with regulatory proteins. For example, binding of 4E-BP to eIF4E inhibits the latter. Similarly, components of the MAPK pathway interact with each other and with MAPK substrates. Likewise, components of the mTOR pathway interact with each other and with substrates. In addition, among the novel findings described herein are the ability of ERK to influence S6K activity and the ability of the mTOR pathway to influence eIF4E phosphorylation, indicating crosstalk between these pathways. Therefore, in accordance with the invention, agents that promote or inhibit interactions (e.g., binding interactions) between pathway components are of use for modulating cognitive function.

Formation of complexes between a protein of interest and one or more other proteins may be detected using a number of methods well known in the art and can be performed in either cell-free or cell-based systems. Methods for detection include immunological methods, chromatographic methods, etc. Frequently it will be desirable to detectably label the protein of interest, e.g., with a fluorescent or radioactive label, and/or to epitope tag the protein of interest.

In many of the assays, the protein of interest or a potential binding protein is immobilized, e.g., in a vessel such as a microtiter plate or microfuge tube, to a chromatographic matrix, etc Immobilization may be accomplished using crosslinking agents or antibodies or by biotinylating the protein and utilizing a vessel to which avidin is attached. In some embodiments a fusion protein comprising a protein of interest and heterologous sequence comprising a binding domain (e.g., GST, 6×-His, maltose binding domain, etc.) is generated. After immobilizing the protein, the protein is contacted with a potential interacting protein (either partially or fully purified, or in a cell lysate, etc.). After a period of incubation, a wash is performed to remove unbound material. Complex formation can be detected using an antibody that binds to the potential interacting protein. Alternately, the interacting protein may be detectably labeled (e.g., enzymatically, fluorescently, etc). The ability of an agent to promote or inhibit complex formation is assessed by allowing complex formation to occur in the presence of the agent (or by adding the agent following complex formation) and comparing the extent of complex formation with that occurring in the absence of the agent. Proteins can also be subjected to various procedures that involve separation based on size. Complex formation results in a detectable increase in size. Methods for detecting the increase in size include chromatography, gel electrophoresis, etc.

Another widely used method for detecting protein-protein interactions is the so-called two-hybrid approach, which is described in U.S. Pat. Nos. 5,283,173; 5,468,614; and 5,667,973. Briefly, the method is based on reconstituting a functional transcriptional activator protein from two separate fusion proteins in a biological system, preferably in living cells in culture, although in vitro biological systems other than intact cells, (optionally containing cellular constituents) could also be employed. This reconstitution makes use of chimeric genes which express fusion proteins. At least one of the fusion proteins contains a protein of interest or a portion thereof. The other fusion protein contains a known or potential interacting protein. Each fusion protein also contains a domain of a transcriptional activator, e.g., one fusion protein contains a DNA binding domain and the other fusion protein contains a transactivation domain. Interaction between the fusion proteins reconstitutes a transcriptional activator, leading to expression of a reporter construct that contains a binding site for the transcriptional activator fused to a sequence that encodes a reporter protein. A variety of suitable reporter proteins are known in the art. Expression of the reporter construct is detected and provides an indication that interaction has occurred. The chimeric gene encoding the fusion protein containing the interacting protein can then be isolated, allowing identification of the interacting protein. Numerous variants and improvements on this method have been made since its initial description and can be employed in the present invention. For example, transcriptional repression domains could also be used, wherein the readout would be reduced expression of the reporter construct if an interaction occurs.

The two hybrid system can also be used to identify agents, e.g., small molecules, that disrupt interaction of two known proteins. In this approach, a transcriptional activator is reconstituted as described above using first and second fusion proteins, each of which contains a domain of a transcriptional activator and one of the known interacting proteins (or a portion thereof). Reconstitution of the transcriptional activator results in expression of a reporter construct, which can be detected. A test agent is added to the biochemical system. If the test agent disrupts the interaction, a decrease in expression of the reporter construct will be detected.

Three hybrid screening assays are also of use in the present invention. These assays are useful for screening chemical libraries, e.g., libraries of small molecules, to identify agents that can bind to particular targets of interest, e.g., components of the general translation machinery, components of the MAPK signaling pathway, etc. Methods and reagents for performing three hybrid screening assays are described in U.S. Pat. No. 5,928,868 and in Licitra et al., Proc. Natl. Acad. Sci. USA 93: 12817-21 (1996).

Briefly, the three hybrid assay involves the formation of a complex between a hybrid ligand and two hybrid proteins in which a portion of a component of the three hybrid complex may be unknown. The unknown component can be either a small molecule that forms part of the hybrid ligand or forms part of one of the hybrid proteins. The three hybrid assay is based on a similar concept to the two hybrid assay described above, i.e., formation of a complex (in this case a three component complex) triggers the expression of a reporter gene. Expression of the reporter gene is detected using a suitable technique and indicates interaction of the members of the complex. The unknown component is then identified.

In the context of the present invention the three hybrid assay can be used for any one or more of the following purposes: (i) determining the identity of a small molecule capable of direct binding to a known target molecule (e.g., a component of the translation machinery or a component of the MAPK signaling pathway) where the identified small molecule may be suitable as a modulator of translation and/or as a modulator of the activity of a component of the MAPK signaling pathway; (ii) determining the identity of a small molecule capable of binding competitively to a known target molecule (e.g., a component of the translation machinery or a component of the MAPK signaling pathway) in the presence of a hybrid molecule so as to inhibit the binding between the target and a second small molecule that forms part of the hybrid molecule (e.g., the second small molecule may be a known ligand for the target molecule); (iii) establishing screening assays, e.g., high throughput assays, in any of a variety of cell types and/or organisms to screen for candidate active agents that modulate translation and/or modulate the MAPK signaling pathway.

### H. Animal Models for Testing Candidate Modulators of Cognitive Function

The screening methods described above offer a number of advantages in that they are rapid and can be used to efficiently screen a large number of substances, often in a quantitative manner. Agents identified using the inventive screening methods may be further tested in a variety of animal models (e.g., rodents, primates) that are commonly employed in the study of learning and memory and in screens to identify compounds of use in the treatment or prevention of memory impairment. Certain appropriate tests for use in rodents are described in (Tang 1999) and include novel-object-recognition tasks, contextual and cued fear conditioning, fear-extinction, eyeblink conditioning test, holeboard test, inhibitory avoidance, visual delay non-match to sample, spatial delay non-match to sample, visual discrimination, Barnes circular maze, Morris water maze and Radial arm maze tests. Additional suitable tests and further details are described in U.S. Pat. No. 6,632,806 and in US Publication No. 20030166555. The ability of a candidate agent to modulate cognitive function can be tested in chicks using, e.g., a taste discrimination test.

In certain embodiments of the invention normal, wild-type animals are used. In other embodiments aged animals (e.g., mice greater than 18 months of age) are used. Animals having lesions in various parts of the central nervous system may be used. Preferably such lesions interfere with one or more aspects of cognitive function. See, e.g., US Publication No. 20030166555. A variety of animal models for stroke are known in the art. Such animals can be used to measure the ability of a candidate agent to improve cognition following brain injury. In certain embodiments a transgenic animal such as those described below is used.

### I. Transgenic Animals

The invention provides a transgenic animal that overexpresses one or more (i) proteins that are components of the general translation machinery; (ii) proteins that regulate the activity or abundance of one or more components of the general translation machinery; and/or (iii) proteins that are components of a MAPK pathway. Such animals may be used, for example, to determine whether overexpression of the component affects cognitive function. If overexpression of the component does affect cognitive function, then the component itself, or agents that mimic overexpression of the component (e.g., agonists of the component or inhibitors of negative regulators of the component) are candidate agents for modulation of cognitive function. For example, if overexpression of the component enhances cognitive function, then the component itself, or agents that increase the activity or mimic overexpression of the component (e.g., agonists of the component or inhibitors of negative regulators of the component) are candidate agents for enhancement of cognitive function.

The invention further provides a transgenic knockout animal in which expression of one or more (i) proteins that are components of the general translation machinery; (ii) proteins that regulate the activity or abundance of one or more components of the general translation machinery; and/or (iii) proteins that are components of a MAPK pathway is prevented by modification of the endogenous gene that encodes the protein (e.g., by insertion of a heterologous sequence into the gene, removal of part of the gene by homologous recombination, etc.). Such animals may be used to determine whether reduced expression of the component affects cognitive function. For example, if reduced expression of the component reduces cognitive function, then the component itself, or agents that increase the activity or mimic overexpression of the component (e.g., agonists of the component or inhibitors of negative regulators of the component) are candidate agents for enhancement of cognitive function. Conversely, if reduced expression of the component enhances cognitive function, then agents that reduce the activity or expression of the component (e.g., antagonists of the component) are candidate agents for enhancement of cognitive function.

The invention further provides a transgenic animal in which one or more (i) proteins that are components of the general translation machinery; (ii) proteins that regulate the activity or abundance of one or more components of the general translation machinery; and/or (iii) proteins that are components of a MAPK pathway is functionally inhibited, e.g., by transgenic expression of a “dominant negative” version of the protein or by transgenic expression of an RNAi agent targeted to a transcript that encodes the protein. Dominant negative versions are inactive versions of a protein (e.g., lacking a modification site that is required for activity, lacking a catalytic domain, having a mutation in an active site etc.). Other means of functionally inhibiting a protein include overexpression of a non-functional substrate, overexpression of an inhibitory protein, etc.

In certain embodiments of the invention transgene expression or knockout of an endogenous gene is conditional, inducible, tissue-specific, or region-specific. For example, a transgene may be overexpressed selectively in a structure or region of the brain. The structure or region may be, for example, the cortex, hippocampus, thalamus, amygdala, etc. The region may be, e.g., the CA1 region of the hippocampus. An endogenous gene may be knocked out selectively in such a structure or region. In certain embodiments, for example, expression of the transgene is limited to certain subsets or types of cells, tissues or developmental stages. This may be achieved by using cis-acting sequences that control expression to achieve a desired spatial, temporal, or developmentally restricted pattern. Conditional recombination systems may be used. Methods for producing transgenic non-human animals are well known in the art. Typically such animals are produced by introducing a transgene into the germline of the animal. Accurate gene targeting is achieved using homologous recombination and selection for desired recombinants.

### J. Testing Candidate Agents in Human Subjects

Candidate agents may be further screened in humans using, for example, any of a variety of tests of memory and/or learning ability such as are widely used in psychology and medicine, e.g., the Clinician's Interview-Based Impression of Change Plus Caregiver Input (CIBIC-Plus), the Alzheimer's Disease Cooperative Study Activities of Daily Living Inventory modified for severe dementia (ADCS-ADLsev), the Severe Impairment Battery, etc. (Reisberg 2003), and various other tests of cognitive function mentioned above. Diagnosis of a condition associated with cognitive impairment, e.g., Alzheimer's disease, dementia, etc., may be performed in accordance with diagnostic criteria set forth in Diagnostic and Statistical Manual of Mental Disorders DSM-IV-TR (Text Revision) American Psychiatric Association; 4th edition (June 2000).

### K. Agents Suitable for Screening and Use

Agents suitable for screening and for use in the compositions and methods of the present invention include small molecules, natural products, polypeptides, peptides, nucleic acids, etc. Sources include natural product extracts, collections of synthetic compounds, and compound libraries generated by combinatorial chemistry. Agents may be selected using phage display technology, aptamer technology, etc.

Libraries of compounds are well known in the art. One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., “Stereoselective Synthesis of Over Two Million Compounds Having Structural Features Both Reminiscent of Natural Products and Compatible with Miniaturized Cell-Based Assays”, Am. Chem. Soc. 120, 8565-8566, 1998; Floyd C D, Leblanc C, Whittaker M, Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. One of ordinary skill in the art will readily be able to identify further sources of compounds to screen. In addition, one of skill in the art of medicinal chemistry will be able to modify compounds in the classes discussed above to develop analogs that exhibit superior properties. Such enhancement may increase the activity of the agent, increase its bioavailability, decrease its metabolism to a less active form, etc.

Molecular modeling can be used to identify a pharmacophore for a particular target i.e., the minimum functionality that a molecule must have to possess activity at that target. Such modeling can be based, for example, on a predicted or known structure for the target (e.g., a two-dimensional or three-dimensional structure). Software programs for identifying such potential lead compounds are known in the art, and once a compound exhibiting activity is identified, standard methods may be employed to refine the structure and thereby identify more effective compounds. Structures of a number of the receptors and other targets described herein are known in the art. In addition, known agonists and/or antagonists can be modified, and the effects of the modified compounds on translation, phosphorylation, synaptic plasticity, memory, etc., can be assessed. Thus any of the above screening methods may be performed using a modified version of an agent identified using one of the inventive screens or otherwise identified as having activity towards one of the targets mentioned herein.

### L. Database

The invention includes a database stored on a computer-readable medium (e.g., a hard disk, floppy disk, compact disk, zip disk, flash memory, magnetic memory, etc.) containing information related to any of the screening methods described above. The database may include descriptions or names of tests performed, lists of compounds tested, amounts used, results of tests, etc. The invention also includes a method comprising the step of electronically sending or receiving information related to any of the screening methods described above.

### VI. Applications

Age-associated decreases in memory have been given a variety of names, including “benign senescent forgetfulness”, “age-associated memory impairment”, “age-associated cognitive decline”, etc. (Petersen 2001; Burns 2002). These terms are intended to reflect the extremes associated with normal aging rather than a precursor to pathologic forms of memory impairment. For example, age-associated memory impairment has been described as requiring performance at least one standard deviation below the performance of young adults on certain tests indicative of memory function. Attention has recently focused on a condition referred to as “mild cognitive impairment” (or, more specifically, “amnestic mild cognitive impairment”). This term describes individuals with memory impairment more severe than those associated with normal aging but who do not meet the criteria for diagnosis of clinically probable AD. These individuals progress to clinically probable AD at an accelerated rate compared with healthy, age-matched controls (Petersen 2001).

The methods and compositions of the invention are suitable for a number of different therapeutic purposes. They may be administered to individuals (subjects) suffering from any of a variety of conditions in which cognitive function, e.g., memory and/or learning is impaired. The compositions are also useful to prevent the onset of such conditions. These conditions include, but are not limited to, those known as “benign senescent forgetfulness”, “age-associated memory impairment”, “age-associated cognitive decline”, “mild cognitive impairment”, Alzheimer's disease, dementias (associated with any of a number of causes), attention-deficit disorder, etc. The compositions and methods of the invention may also find use to enhance the cognitive function, e.g., memory and/or learning capacity of normal individuals, i.e., individuals not suffering from any clinically recognized condition or disorder. They may be useful on a short-term basis or may be administered chronically. They may be administered daily, multiple times per day, or at intervals greater than a day.

In addition, it has been observed that in its early stages Alzheimer's disease characteristically features an impairment of memory and a relative absence of other symptoms of cognitive malfunction that typically occur later in the disease. Mounting evidence suggests that this syndrome begins with subtle alterations of hippocampal synaptic efficacy prior to frank neuronal degeneration (Selkoe 2002).

Also within the scope of the invention is treatment of various conditions associated with cognitive impairment due to causes such as stroke (either ischemic or hemorrhagic), neoplastic disorders of the CNS, degenerative conditions, or any other condition in which enhanced plasticity and/or memory is desired.

Accordingly, the invention provides a method of modulating cognitive function in a mammalian subject comprising steps of: (i) providing a mammalian subject in need of modulation of cognitive function; and (ii) administering to the subject a composition comprising an agent that modulates translation. The invention further provides a method of modulating cognitive function in a mammalian subject comprising steps of: (i) providing a mammalian subject in need of modulation of cognitive function; and (ii) administering to the subject a composition that modulates a MAPK signaling pathway. “Administering”, as used herein, refers to prescribing the composition or making the composition to available to the subject for self-administration in addition to, or instead of physically introducing the composition into or onto the subject's body.

Preferred compositions enhance cognitive function. The compositions may be administered for short periods of time such as days or a few weeks, e.g., to provide short term enhancement of learning ability or memory. However, it is anticipated that the inventive compositions may be administered on a chronic basis, e.g., for many weeks, for months, for years, or indefinitely. The subject may be suffering from or at risk of memory impairment from any of a variety of causes. In particular, the subject may be at risk of or suffering from age-associated memory impairment, mild cognitive impairment, or Alzheimer's disease. The subject may have suffered a brain injury, e.g. from trauma or stroke, may have amnesia (i.e., a specific defects in declarative memory) or may have a developmental disorder associated with impaired cognitive ability. Amnesia may be long-lasting or of short duration. Various types of amnesia and causes thereof are known and may be treated in accordance with the invention, e.g., amensias due to alcohol or drug intoxication, seizures, migraines, trauma, infection, etc.

The agents and methods are generally useful for modulating one or more aspects of cognitive function in a subject. The methods of the invention may be applied to modulate any aspect of cognitive function, e.g., learning and/or memory, within a subject. For example, the methods may be applied to a prevent or reduce loss of stored memories, to treat an inability or reduced ability to form new memories, to enhance memory formation, etc. The subject may be a patient at risk of or suffering from a condition or disorder associated with memory impairment, such as those mentioned above. The compounds may be administered during all or part of the period during which enhancement is desired. Preferably the compounds are administered at intervals during the time over which enhancement is desired. For example, the compounds can be administered 3-4 times daily, 1-2 times daily, every other day, weekly, etc. It may be preferred to maintain an effective concentration within the body over a time period during which cognitive enhancement is desired. Since, in general, it is desirable to maintain cognitive function throughout life, the compounds may be administered indefinitely.

Without wishing to be bound by any theory, enhancement of translation, cognitive function, and/or synaptic plasticity may be facilitated by neuronal activity. Therefore certain embodiments of the invention involve stimulating neuronal activity in a subject to whom an agent of the invention is administered. In certain embodiments of the invention neuronal activity is stimulated in conjunction with administration of an agent of the invention, e.g., an agent that modulates translation, an agent that modulates a MAPK signaling pathway, etc. Neuronal activity can be stimulated in a variety of ways. For example, electrical stimulation (e.g., using implanted electrodes) or chemical stimulation can be employed. In certain embodiments of the invention the subject to whom a composition of the invention is administered is engaged in a program of rehabilitative therapy or training. The program may stimulate neuronal activity in one or more regions of the nervous system. Such programs are typically a part of therapy after injury or stroke, but also include programs of remediation and training in a variety of disorders of developmental or adult onset, e.g., dyslexia, autism, Asperger's Syndrome, Pervasive Developmental Disorders—Not Otherwise Specified, Tourette's Syndrome, Personality Disorders, Schizophrenia and related disorders. See, e.g., Diagnostic and Statistical Manual of Mental Disorders, 4th Ed. (DSM-IV) (American Psychiatric Association. (1994) Diagnostic and Statistical Manual (Am. Psychiatric Assoc., Washington, D.C.) for discussion of these disorders. Numerous rehabilitation programs for victims of stroke, spinal cord injury, and other forms of nervous system damage, are known to those skilled in the art, and the subject can be engaged in any such program. See, e.g.,; Gillen, G. and Burkhardt, A. (eds.), Stroke Rehabilitation: A Function-Based Approach, 2^{nd} ed., C. V. Mosby, 2004, for a discussion of suitable programs for victims of stroke. Similar programs may be used for victims of other forms of damage to the brain. The program may entail a practice regimen that engages one or more specific regions in the brain, e.g., a region that suffered damage, a region involved in memory formation or consolidation, etc.

In certain embodiments of the invention the agent is administered or released in a defined temporal relation to stimulation and/or rehabilitative therapy, e.g., during, prior to, or following neuronal stimulation and/or engagement of the subject in one or more rehabilitative activities. The agent may, for example, be administered up to 5 minutes-12 hours prior to the activity, up to 5 minutes-12 hours after the activity, during the activity, or immediately prior to or immediately following neuronal stimulation or the start of a therapy session, e.g., up to 5 minutes prior to the beginning of a therapy session or up to 5 minutes following the start of a therapy session. By “therapy session” is meant any period of time in which the subject is engaged in performing activities that have been suggested or prescribed by a health care provider for purposes of assisting the recovery and/or improvement of the subject's cognitive function and/or memory. The health care provider need not be present during the therapy session, e.g., the subject may perform the activities independently or with the assistance of personnel other than a health care provider.

A variety of criteria may be used to determine whether or not a particular individual suffers from or is at risk of developing any of the conditions discussed herein. See, e.g., discussion in (Petersen 2001; Burns 2002; Clark and Karlawish 2003; and Karlawish and Clark 2003) and references listed in these articles. In particular, AD may be diagnosed according to the National Institute of Neurological and Communicative Disorders and Stroke—Alzheimer's Disease and Related Disorders Association criteria for a clinical diagnosis of probable Alzheimer's disease. Imaging and various biomarkers (e.g., levels of tau protein in cerebrospinal fluid). In addition, individuals with dominant mutations in the amyloid precursor protein, PS1, or PS2 genes are at increased risk of AD. It has also been found that the risk of developing AD is greater in individuals with the ε4 allele of the gene encoding ApoE. Such individuals may be particularly appropriate candidates for therapy with the compositions described herein.

In addition to the various therapeutic applications described above, the agents and methods may also be used for enhancing cognition, L-LTP, and/or synaptic plasticity in an animal or in a biological system such as hippocampal slice or a cultured neuronal system for research purposes, to compare the effects with those of accepted treatments.

### VII. Pharmaceutical Compositions and their Administration

Compositions of this invention may be formulated for delivery by any available route including, but not limited to oral, parenteral, intradermal, subcutaneous, by inhalation, transdermal (topical), transmucosal, rectal, and vaginal. The term “parenteral” as used herein includes subcutaneous, intravenous, intramuscular, intra-articular, intra-synovial, intrasternal, intrathecal, intrahepatic, intralesional and intracranial injection or infusion techniques. Inventive pharmaceutical compositions typically include one or more compounds of the classes discussed above in combination with a pharmaceutically acceptable carrier, adjuvant, or vehicle. As used herein the language “pharmaceutically acceptable carrier, adjuvant, or vehicle” includes solvents, dispersion media, coatings, antibacterial and antifungal agents, isotonic and absorption delaying agents, and the like, compatible with pharmaceutical administration. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Solvents, dispersion media, coatings, antibacterial and antifungal agents, isotonic and absorption delaying agents, and the like, compatible with pharmaceutical administration may be included. Supplementary active compounds, e.g., compounds independently active against the disease or clinical condition to be treated, or compounds that enhance activity of a compound that is active against the disease or clinical condition being treated, can also be incorporated into the compositions. Compounds useful for modulating translation and/or for modulating activity or expression of one or more components of the MAPK signaling pathway may be administered as single agents or in combination. For example, it may be desirable to activite multiple components of the MAPK signaling pathway. If administered in combination they may be administered individually or as part of a single composition. If administered individually they may be administered sequentially or concurrently.

Further provided are pharmaceutically acceptable compositions comprising a pharmaceutically acceptable derivative (e.g., a prodrug) of any of the compounds of the invention, by which is meant any non-toxic salt, ester, salt of an ester or other derivative of a compound of this invention that, upon administration to a recipient, is capable of providing, either directly or indirectly, a compound of this invention or an active metabolite or residue thereof. As used herein, the term “active metabolite or residue thereof” means that a metabolite or residue thereof is also able to enhance cognitive function and/or synaptic plasticity.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, chloride, bromide, iodide, hydrochloride, hydrobromide, hydroiodide, hydroxide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C1-4 alkyl)4 salts. This invention also envisions the quaternization of any basic nitrogen-containing groups of the compounds disclosed herein. Water or oil-soluble or dispersible products may be obtained by such quaternization.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. A parenteral preparation can be enclosed in ampules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive compositions are preferably delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from neuronal culture assays. A dose can be formulated in animal models to achieve a circulating plasma concentration range that includes the IC₅₀ (i.e., the concentration of the test compound which achieves a half-maximal inhibition of symptoms) as determined in neuronal cell culture. Such information can be used to more accurately determine useful doses in humans. Levels in plasma can be measured, for example, by high performance liquid chromatography.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. For certain conditions it may be necessary to administer the therapeutic composition on an indefinite basis to keep the disease under control. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an inventive composition as described herein, can include a single treatment or, in many cases, can include a series of treatments.

Exemplary doses include milligram or microgram amounts of the inventive composition per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) It is furthermore understood that appropriate doses may optionally be tailored to the particular recipient, for example, through administration of increasing doses until a preselected desired response is achieved. It is understood that the specific dose level for any particular subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

In the case of compounds that are currently approved for use in humans (e.g., compounds approved by the U.S. Food and Drug Administration or a comparable agency in another country), in certain embodiments of the invention the dose of such a compound in the compositions of the present invention is preferably less than or equal to the maximum tolerated therapeutic dose of the compound for the treatment of diseases or conditions in which its clinical efficacy is recognized. In certain embodiments of the invention, the dose of such a compound in the inventive compositions may be less than the minimum recommended therapeutic dose of the compound, less than 90%, 80%, 70%, 60%, 50%, 40%, 30%, 20%, 10%, or 5% of the minimum recommended dose. One of ordinary skill in the art will readily be able to determine the maximum tolerated dose and/or minimum recommended dose of any given compound by consulting works such as the Physician's Desk Reference, pharmacology books such as Goodman and Gilman, supra, and/or the scientific/medical literature. Furthermore, it will be appreciated that total dose depends both on the amount of compound that is administered at each dosing and on the interval between doses. Therefore, when a compound useful in the practice of the present invention is administered at a dosing interval that differs from the dosing intervals typically employed when the compound is used for therapy of a disease or condition in which its efficacy has been recognized, the amount of each dose may be adjusted so that the total dose is less than the maximum tolerated dose of the compound.

It will further be appreciated that where compositions comprising multiple different compounds are used, it will generally be desirable to limit the total dose of the composition in addition to limiting the doses of individual constituents of the composition.

Inventive pharmaceutical compositions can be included in a container, pack, or dispenser together with instructions for administration.

The invention includes a unit dosage form of any of the compounds referred to herein (or combinations thereof), wherein the unit dosage is selected to modulate translation so as to modulate cognitive function in a desired manner. The invention further includes a unit dosage form of any of the compounds referred to herein, wherein the unit dosage is selected to modulate activity of the MAPK signaling pathway or a component thereof so as to modulate cognitive function in a desired manner.

In certain embodiments of the invention a pharmaceutical composition is delivered locally to the CNS, e.g., by implantation, injection, intrathecal catheter, implantable or external pump, surgery, etc. For example, the agent may be incorporated into a biocompatible polymeric matrix, which is preferably biodegradable. The resulting drug delivery device is delivered to or implanted into the body within the CNS, e.g., within the brain. The agent is released from the polymeric matrix over a period of time, e.g. by diffusion out of the matrix or release into the extracellular environment as the matrix degrades. Methods for incorporating therapeutically active agents including proteins and peptides into polymeric matrices are known in the art, and a number of different agents have been delivered to the CNS using such matrices.

Preferably the polymeric matrix is made of a biodegradable material, by which is meant a material capable of being broken down physically and/or chemically within the body of a subject, e.g., by hydrolysis under physiological conditions, by natural biological processes such as the action of enzymes present within the body, etc., to form smaller chemical species which can be metabolized and/or excreted. Suitable biocompatible, biodegradable polymers include, for example, poly(lactides), poly(glycolides), poly(lactide-co-glycolides), poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetals, polycyanoacrylates, polyetheresters, poly(dioxanone)s, poly(alkylene alkylates)s, polyhydroxyalkanoates, poly(glycerol-sebacate)s, copolymers of polyethylene glycol and polyorthoesters, biodegradable polyurethanes, blends and copolymers thereof. The material, and methods used in making the implant, should be compatible with protein stability. For purposes of the present invention, any method that achieves delivery of an agent to the CNS, particularly intracranially, without requiring transport via the vascular system from a site outside the skull or meninges (the membranes that cover the brain and the spinal cord), is considered to achieve local delivery of the agent.

In certain embodiments of the invention an agent or method that increases the permeability of the blood-brain barrier is used in conjunction with administration of an agent that modulates cognitive function. Targeted delivery may also be used.

In certain embodiments of the inventions gene therapy is used to modulate translation. Gene therapy encompasses delivery of nucleic acids comprising templates for synthesis of a therapeutic molecule, e.g., a therapeutic polynucleotide or polypeptide, to a subject. The nucleic acid (or a nucleic acid derived from the nucleic acid as, for example, by reverse transcription) may be incorporated into the genome of a cell or remain permanently in the cell as an episome. However, gene therapy also encompasses delivery of nucleic acids that do not integrate or remain permanently in a cell to which they are delivered. Such approaches permit temporary or transient synthesis of a molecule of interest.

Therapeutic nucleic acids may reduce expression of a target gene, e.g., a gene whose expression product inhibits or reduces translation or negatively regulates a component of the general translation machinery. In certain embodiments of the invention the target gene is one whose expression product is a component of the translation machinery or activates or enhances translation. Such nucleic acids include, but are not limited to, RNAi agents, antisense oligonucleotides, and ribozymes. For example, the nucleic acid may, but need not be, be a vector such as an RNAi vector.

Antisense nucleic acids are generally single-stranded nucleic acids (DNA, RNA, modified DNA, modified RNA, or peptide nucleic acids) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Antisense technology and its applications are well known in the art and are described in Phillips, M. I. (ed.) Antisense Technology, Methods Enzymol., Volumes 313 and 314, Academic Press, San Diego, 2000, and references mentioned therein. See also Crooke, S. (ed.) “Antisense Drug Technology: Principles, Strategies, and Applications” (1^{st} ed), Marcel Dekker; ISBN: 0824705661; 1st edition (2001) and references therein.

Ribozymes (catalytic RNA molecules that are capable of cleaving other RNA molecules) represent another approach to reducing gene expression. Such ribozymes can be designed to cleave specific mRNAs corresponding to a gene of interest. Their use is described in U.S. Pat. No. 5,972,621, and references therein. Extensive discussion of ribozyme technology and its uses is found in Rossi, J. J., and Duarte, L. C., Intracellular Ribozyme Applications: Principles and Protocols, Horizon Scientific Press, 1999.

Nucleic acids can be delivered to a subject as a pharmaceutical composition, e.g., with pharmaceutically acceptable carriers, excipients, etc. In certain embodiments a nucleic acid is complexed with a delivery-enhancing or stabilizing component, e.g., a biocompatible, preferably biodegradable polymer such as those mentioned above.

In general, gene therapy involving administration of a nucleic acid encoding a particular protein may be used similarly to administration of an agonist of the protein as described herein for modulation of cognitive function. Similarly, gene therapy involving administration of a nucleic acid that reduces expression of a target gene may be used similarly to administration of an antagonist of the protein encoded by the gene as described herein for modulation of cognitive function.

### EXAMPLES

### Example 1

### Generation and Characterization of Conditional Transgenic Mice Expressing Dominant-Negative MEK1 in the Postnatal Forebrain

Materials and Methods

Plasmid constructions. The conditional transgene vector pCLSL contains a floxed transcriptional and translational ‘stop’ cassette (Lakso et al., 1992) inserted downstream of promoter sequences derived from the chicken β-actin gene. A dominant-negative MEK1 cDNA bearing a K97M mutation and the SV40 late polyadenylation signal was derived from pMCL-dnMEK1 (gift of N. Ahn) and inserted downstream of the stop cassette in pCLSL to generate pCLSL-dnMEK1. pCMV-EGFP-CKUTR was derived from pEGFP-N1 by deletion of the SV40 polyadenylation signal and insertion of a 160-bp PCR fragment encoding the distal sequences of the αCaMKII 3′ UTR (including both CPEs and the hexamer sequence (Wu et al., 1998)). The αCaMKII 3′ UTR fragment was amplified from a rat brain Marathon cDNA library (Clontech). CPE mutations were introduced using previously described primers (Wu et al., 1998). The hexamer mutation AAGAAA was similarly introduced by PCR.

Generation of dnMEK1 Mice. dnMEK1 transgenic mice were prepared by pronculear injection of the linearized dnMEK1 transgenic construct, derived from pCLSL-dnMEK1 by excision of the vector backbone, in C57BL/6J embryos, using standard methods.

In situ hybridization. Transgene expression was analyzed using a ³³P-labeled RNA oligonucleotide specific for the transgene 5′ UTR. In situ hybridization was performed on sagittal cryosections as described (Zeng et al., 2001). Fluorescent in situ hybridization of hippocampal neurons was performed with a digoxigenin-labeled cRNA probe derived from the EGFP coding region. Fluorescence intensities were quantified with ImageJ (NIH). Relative mRNA levels were expressed as the mean fluorescence intensity of randomly selected neurons.

Western analysis. Homogenates were prepared in cold RIPA buffer containing protease inhibitors and phosphatase inhibitors. Hippocampal neurons were homogenized 8 minutes after stimulation. Hippocampal slices were frozen on dry ice 10 minutes after tetanization, and the CA1 and CA3 fields were microdissected and homogenized. Western analysis was performed with rabbit polyclonal antisera against dually-phosphorylated ERK1/2, phospho-S6 (S235/S236), phospho-eIF4E (S209), and phospho-4E-BP (S65) (Cell Signaling). The antibody did not distinguish between 4E-BP1, 4E-BP2, and 4E-BP3. Therefore, since 4E-BP2 is the most abundantly expressed 4E-BP in the brain, it is likely that the results reflected 4E-BP2 levels and phosphorylation state. Blots were stripped and reprobed with antisera directed against total ERK1/2, S6, eIF4E, and 4E-BP (Cell Signaling). Results were quantified with ImageJ (NIH), calculated as the ratio of phosphorylated species to total ERK, and then normalized to the untreated control condition Staining of total protein with Ponceau-S confirmed equal loading.

Results

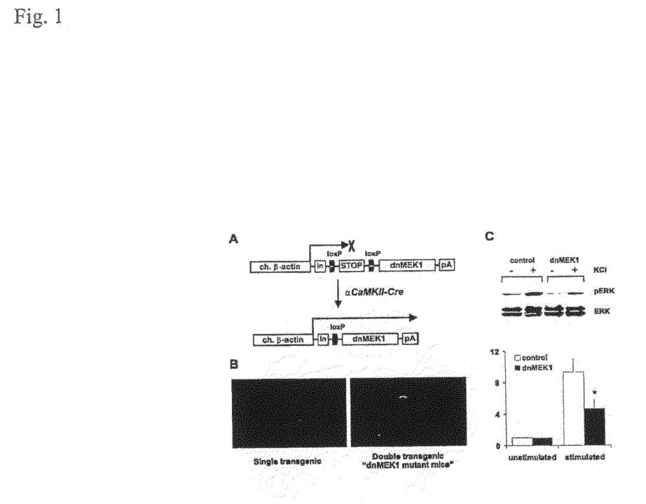

To investigate the possible involvement of ERK signaling in the protein synthesis-dependent phases of memory and LTP, we generated mutant mice in which a dominant-negative form of the ERK kinase MEK1 (dnMEK1) is expressed selectively in the postnatal forebrain (FIG. 1A). This dominant-negative form of MEK1 bears a K→M substitution in the ATP-binding site, abolishing its kinase activity but preserving its ability to interact with ERK1 and 2, thereby inhibiting their MEK-dependent activation (Mansour et al., 1994). The conditional approach to restrict the spatiotemporal pattern of transgene expression required the generation of transgenic mice in which a floxed (flanked by loxP sites) transcriptional/translational stop cassette prevents expression of the dnMEK1 cDNA from the chicken β-actin promoter. In the absence of Cre recombinase, the transgene was not detectably expressed in the brains of these “foxed” single transgenic mice (FIG. 1B, left). Floxed single transgenic mice were then crossed to αCaMKII-Cre transgenic mice previously shown to mediate preferential excision of floxed sequences in a subset of excitatory neurons in the postnatal forebrain (Tsien et al., 1996; Zeng et al., 2001). Expression of the dnMEK1 transgene in the brains of the resulting double transgenic mice (designated “dnMEK1 mice”) was largely restricted to hippocampal area CA1 and the neocortex (FIG. 1B, right). Consistent with the prior reports employing the same αCaMKII-Cre mice, expression was undetectable in hippocampal area CA3 (Tsien et al., 1996; Zeng et al., 2001).

Although ERK signaling has been reported to promote the survival of cultured neurons under conditions of nutrient or growth factor deprivation (Xia et al., 1995), we detected no evidence of compromised neuronal survival in dnMEK1 mice (data not shown). To examine the effects of transgene expression on ERK activation in the brains of adult transgenic mice, we stimulated acute hippocampal slices from control and mutant mice with membrane depolarization, a procedure shown to result in robust ERK activation (Wu et al., 2001). Stimulation produced strong ERK activation in control slices, as measured by levels of dually-phosphorylated ERK1/2 (FIG. 1C), but levels of ERK activation were significantly reduced in mutant slices. We subsequently found that ERK activation was also significantly reduced in the dnMEK1 hippocampus in response to L-LTP induction and contextual memory formation (see below).

### Example 2

### Inhibiting ERK Signaling Causes Impaired Spatial Reference Memory

Materials and Methods

Mouse behavioral studies. The Morris water maze and fear conditioning tasks were conducted essentially as described (Tsien et al., 1996; Zeng et al., 2001). The training phase for the hidden platform task of the Morris water maze consisted of two blocks of four 60-sec. trials per day for a total of five consecutive days. Probe trials (60 sec.) were administered following the completion of training. The training sessions for contextual and cued fear conditioning consisted of a three-minute exploration period followed by three CS-US pairings separated by one minute each (foot-shock intensity 0.75 mA, duration 0.5 sec; tone 75 db white noise, 30-sec. duration.). Context tests were performed in the training chamber after retention delays of 60 minutes and 24 hours. Tone tests were performed in a distinct chamber located in a different room; baseline freezing was monitored (2 min.) prior to phasic presentation of the tone (75 db white noise, 3 min. duration). Control groups contained equivalent numbers of single transgenic ‘foxed’ and αCaMKII-Cre mice. Control and mutant groups consisted of age-matched littermates (12-20 weeks of age) for each analysis.

Results

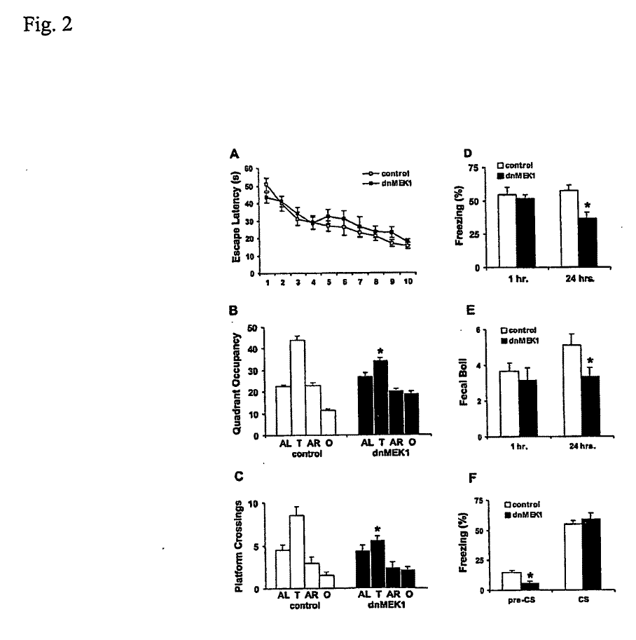

To determine whether inhibition of ERK activation in the hippocampus and neocortex compromises long-term learning and memory, we subjected single transgenic control and double transgenic mutant mice to the hidden-platform version of the Morris water maze, a hippocampus-dependent reference memory task (Morris et al., 1982). The performance of both control and mutant mice improved during the course of training, with a trend toward longer escape latencies in the mutant group (FIG. 2A). Since escape latencies are an insensitive measure of reference memory, probe trials were performed upon the completion of training. Both groups displayed a preference for the pool quadrant in which the platform was located during training, but the mutant mice spent significantly less time than control mice searching in the target quadrant (FIG. 2B). In addition, mutant mice were significantly less accurate in identification of the precise platform location, as indicated by a reduced number of platform crossings (FIG. 2C). No significant differences in swimming speed (controls 16.6±0.8 cm/sec., mutants 16.7±0.7 cm/sec., p>0.05) or thigmotaxis (swimming near the pool perimeter; controls 30.9±6.0%, mutants 30.8±3.8%, p>0.05) were observed between the two groups in the hidden-platform task, and both groups performed similarly in the visible platform version of the task (escape latency, controls 9.5±1.5 sec., mutants 10.5±1.4 sec., p>0.05), indicating that the impairments observed in mutant mice reflect a specific spatial memory deficit.

### Example 3

### Inhibiting ERK Signaling Causes Selective Impairment in Long-Term Contextual Memory

Materials and Methods. See Example 2.

Results

To examine the process of memory consolidation more closely, we turned to contextual fear conditioning, a hippocampus-dependent behavioral paradigm in which robust long-term memory for an experimental context is established following a single training session (Kim and Fanselow, 1992; Phillips and LeDoux, 1992). Administration of protein synthesis inhibitors prior to fear conditioning in rodents has been shown to disrupt long-term memory within 24 hours following training, while short-term memory remained intact (Abel et al., 1997; Schafe et al., 1999). Groups of control and mutant mice were therefore subjected to contextual fear conditioning and tested for memory of the experimental context after retention delays of one hour and 24 hours. Both control and mutant mice exhibited equivalent levels of freezing during the training session and after a one-hour retention delay. In contrast, mutant mice exhibited significantly reduced levels of freezing after a retention delay of 24 hours (FIG. 2D; controls 57.5±4.1%, mutants 36.6±4.7%, p<0.05). Similar results were obtained when defecation was monitored as an independent measure of conditioned fear (FIG. 2E) (Antoniadis and McDonald, 2000; Godsil et al., 2000).

We then evaluated the responses of control and mutant mice to cued fear conditioning, a hippocampus-independent version of the task in which a tone constitutes the conditioned stimulus. When tested in a distinct context after a 48-hour retention delay, mutant mice exhibited normal associative memory for the tone (FIG. 2F). Consistent with their impairment in contextual memory consolidation, the low level of freezing prior to tone presentation was also reduced in mutant mice. A similar reduction in low-level contextual generalization has been observed as a consequence of pre-training hippocampal lesions and anisomycin infusion (Abel et al., 1997; Frankland et al., 1998). Importantly, the normal short-term contextual memory and normal long-term noncontextual memory exhibited by dnMEK1 mice exclude the possibility of any general defect in fear responses. These findings demonstrate a specific impairment in the protein-synthesis dependent phase of hippocampus-dependent contextual memory in dnMEK1 mice.

### Example 4

### Inhibiting ERK Signaling Causes Selective Impairment in the Translational Component of Hippocampal L-LTP

Materials and Methods

Electrophysiology. Transverse hippocampal slices were prepared from age-matched littermates (8-16 weeks of age) and maintained in an immersion chamber perfused with oxygenated artificial cerebrospinal fluid (Kang et al., 2001). Extracellular fEPSPs were evoked by stimulation of the Schaeffer collateral pathway afferents and were recorded in the CA1 stratum radiatum. For LTP studies, stimulation was applied at 0.033 Hz using an intensity that produced ˜35% of the maximal fEPSP slope. Tetanic stimulation was delivered in 1 sec. trains at 100 Hz, with 2 trains separated by 30 sec. used to induce E-LTP, and 4 trains separated by 5 minutes each used to induce L-LTP. Anisomycin (40 μM) and actinomycin-D (40 μM) were added to the perfusate 30 minutes prior to tetanization.

Primary hippocampal neuronal culture and reporter mRNA transfection. High-density hippocampal pyramidal cell (CA1-CA3) cultures were prepared from P1-P2 rat pups as previously described (Liu and Tsien, 1995). Capped reporter mRNAs were synthesized in vitro using T7 Message Machine (Ambion). Templates were generated by PCR amplification of EGFP-αCaMKII 3′ UTR fragments from pCMV-EGFP-CKUTR using primers encoding an upstream T7 promoter and downstream oligo(dT) stretches of specific lengths. Reporter mRNA generated by enzymatic tailing contained >150 poly(A) residues. Reporter mRNAs were transfected on DIV 8 (TransMessenger reagent, Qiagen). Neurons were pre-treated with pharmacological inhibitors (1 μM tetrodotoxin, 100 μM APS, 10 μM DNQX, 20 μM U0126) for 12 hours prior to transfection. Stimulations (100 ng/mL BDNF for 4 hours; 40 μM bicuculline for 8 min.; 90 mM KCl for 3 min. 4 times spaced by 10 min.) were applied immediately following transfection. Coverslips were fixed for analysis 4 hours following transfection. Reporter translation was quantified as the total number of EGFP-positive neurons.

Results

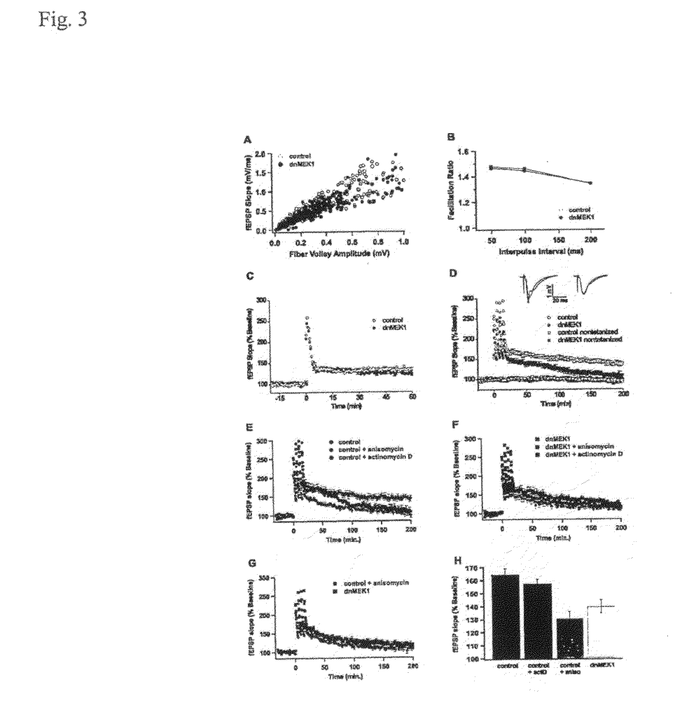

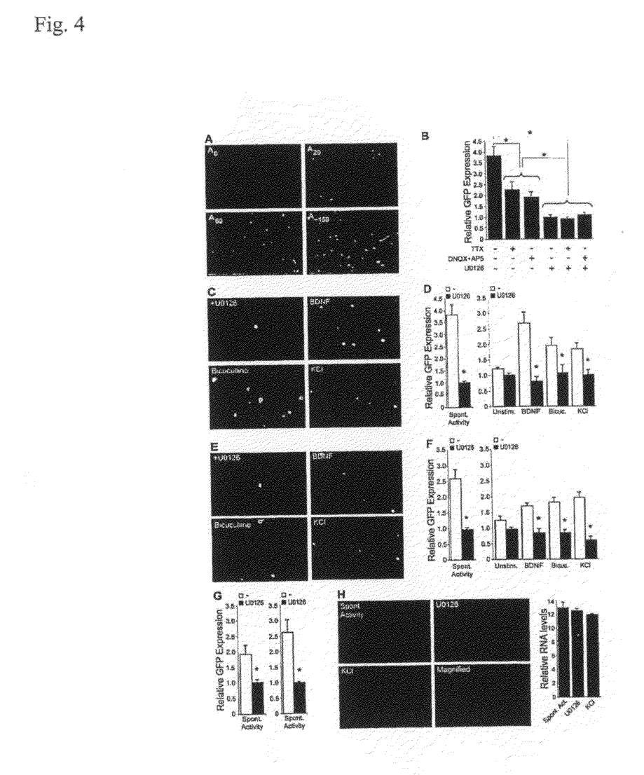

We next investigated hippocampal synaptic transmission and LTP at Schaeffer collateral (SC)—CA1 synapses, hypothesizing that defects in L-LTP might be associated with impaired hippocampal memory consolidation in dnMEK1 mice. Any observed impairments at these synapses should be referable to the postsynaptic neurons, since the dnMEK1 transgene is expressed in area CA1 neurons but not in area CA3 neurons. Basal synaptic transmission was normal in mutant mice, as evidenced by similar synaptic input-output relationship in control and mutant slices (FIG. 3A). Paired-pulse facilitation (PPF), a presynaptic form of short-term synaptic plasticity, was also normal in mutant mice at multiple interpulse intervals (FIG. 3B).

LTP was next induced with two trains of tetanic stimulation separated by 20 seconds, a procedure that induces protein synthesis-independent E-LTP (Winder et al., 1998). Stable potentiation was induced in both the control and mutant groups, with the magnitude of potentiation essentially identical throughout the 60-minute recording (FIG. 3C; E-LTP magnitude at 30 minutes post-tetanization, controls 131.3±4.6%, mutants 128.1±3.2%, p>0.05). We then applied four trains of tetanic stimulation separated by 5-minute intervals, a protocol that induces long-lasting, protein synthesis-dependent L-LTP (Huang and Kandel, 1994). This procedure elicited long-lasting potentiation in control slices that persisted for at least 3 hours after the onset of tetanization. In contrast, mutant slices exhibited an unstable potentiation that progressively decayed throughout the duration of recording, with fEPSP slopes returning near unstimulated levels by 3 hours post-tetanization (FIG. 3D; L-LTP magnitude at 200 minutes, controls 138.6±5.1%, mutants 108.8±4.8%; p<0.05).

Previous publications have suggested that transcriptional and translational inhibitors produce distinct kinetic patterns of L-LTP impairment, with transcriptional inhibition producing a delayed decay of L-LTP (typically beginning more than one hour after the onset of tetanization), and translational inhibition producing an early, progressive decay of L-LTP (Frey et al., 1996; Frey et al., 1988; Frey and Morris, 1997; Nguyen et al., 1994). The kinetics of L-LTP decay in mutant mice strongly resembled the reported effects of translational inhibition on L-LTP. To examine this relationship more closely, we performed an additional series of L-LTP experiments with the transcriptional inhibitor actinomycin-D and the translational inhibitor anisomycin. Consistent with prior reports, treatment of control slices with anisomycin prior to tetanization caused a progressive inhibition of L-LTP similar to that observed in mutant slices, whereas treatment of control slices with actinomycin-D produced a distinct, delayed pattern of inhibition (FIG. 3E). Specifically, L-LTP magnitude in slices treated with actinomycin-D remained indistinguishable from that in untreated slices for approximately 75 minutes after the onset of tetanization (LTP magnitude at 60 min. post-tetanization, untreated controls 164±5%, actinomycin-D-treated controls 157±4%, p>0.05), followed by a progressive decay to anisomycin-treated levels thereafter (LTP magnitude at 200 minutes, untreated controls 143±6%, actinomycin-D-treated controls 107±4%, anisomycin-treated controls 108±5%, p<0.05 for treated relative to untreated conditions). This difference in the kinetic patterns of inhibition by actinomycin-D and anisomycin defines a transcription-independent, translation-dependent phase of L-LTP during the first 60-90 minutes following tetanization.

The inhibitory effects of both agents were occluded in mutant slices, as treatment with either actinomycin-D or anisomycin did not produce any additional decrement in L-LTP (FIG. 3F; L-LTP magnitude at 200 minutes, untreated mutants 115±6%, actinomycin-D-treated mutants 122±5%, anisomycin-treated mutants 113±5%, p>0.05). These results indicate the presence of a translational defect in mutant slices, since a transcriptional defect alone would not have occluded the inhibitory effect of anisomycin. Further supporting this interpretation, the kinetics of L-LTP decay were indistinguishable between the anisomycin-treated control group and the untreated mutant group throughout the duration of the recording (FIG. 3G; L-LTP magnitude at 200 minutes, anisomycin-treated controls 108±5%, untreated mutants 115±6%, p>0.05). In contrast, the time course of L-LTP during the transcription-independent, translation dependent phase differed significantly between the actinomycin-D-treated control and the untreated mutant groups (FIG. 3H; L-LTP magnitude at 60 minutes, actinomycin-D-treated controls 157±3%, untreated mutants 139±5%, p<0.05). Taken together, these results demonstrate maximal blockade of the translational component of L-LTP in dnMEK1 mice.

### Example 5

### ERK Signaling Regulates Translation in Response to Multiple Forms of Neuronal Activity through a Polyadenylation-Independent Mechanism

Materials and Methods. See Example 1.

Results

To investigate a possible role for ERK activation in the regulation of neuronal protein synthesis, we developed a translation reporter assay in cultured primary hippocampal neurons. This assay relies on transfection of cultured neurons with synthetic mRNAs, which permits isolation of translational regulation from regulation of transcription or mRNA processing and transport. Furthermore, this method allows an assessment of the contribution of cis-acting elements and the polyadenylation state of transfected mRNAs to translational regulation. Synthetic reporter mRNAs contained an EGFP reporter coding sequence, preceded by a 5′ m⁷GpppG cap and a ˜30 nt synthetic 5′ UTR. Given the important role of αCaMKII in synaptic plasticity and the proposed role of its 3′ UTR in translational regulation (Soderling, 2000), the EGFP sequences in our reporter mRNAs were appended with a distal ˜160 nt segment of the αCaMKII 3′ UTR (Wu et al., 1998), which contained both CPE elements as well as the hexamer sequence (AAAUAA) required for polyadenylation.

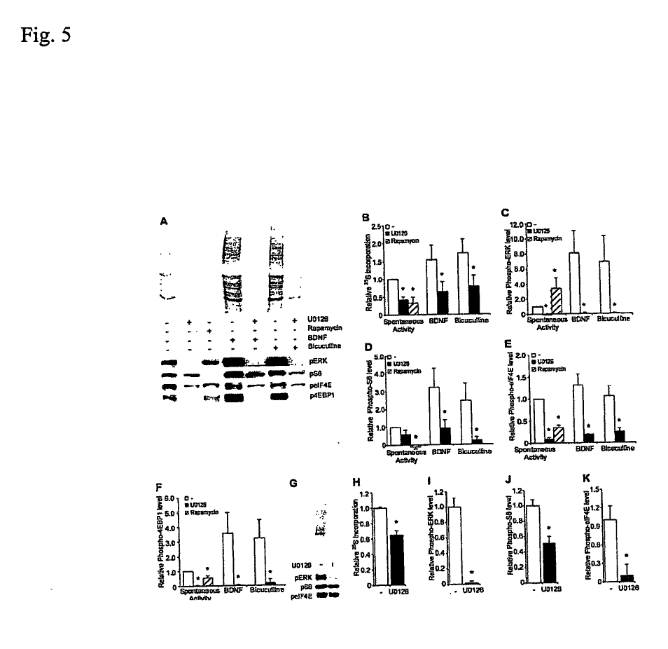

Translation of the transfected EGFP-αCaMKII 3′ UTR reporter mRNA was absolutely dependent on the presence of a minimal poly(A) tail (20 nt), and translational efficiency was progressively enhanced by increasing poly(A) tail lengths (FIG. 4A). In order to bypass any minimal requirement for polyadenylation, we then analyzed the neuronal activity-dependent translation of reporter mRNA appended with a 20-nt poly(A) tail. Moderate levels of basal translation of EGFP-αCaMKI₁-A₂₀ mRNA were observed in the presence of spontaneous neuronal activity, but translation was significantly inhibited by pretreatment with tetrodotoxin (TTX) or the ionotropic glutamate receptor antagonists AP5 and DNQX (FIG. 4B). Reporter mRNA translation was still more strongly inhibited by pretreatment with the specific MEK inhibitor U0126, which blocks ERK activation (FIG. 4B). This effect was specific to the pharmacologic action of U0126 on MEK, since the inactive conformer U0124 had no significant effect (data not shown). Combined pretreatment with U0126 and either TTX or AP5/DNQX produced no additional inhibition (FIG. 4B), suggesting that the translational effects of spontaneous neuronal activity may be largely mediated through the ERK signaling pathway. To evaluate the effects of different forms of neuronal activity, we stimulated hippocampal neurons with BDNF, the GABA-A receptor inhibitor bicuculline and membrane depolarization. In order to reduce background EGFP expression, neurons were incubated with U0126 for 12 hours and then washed just prior to transfection. All three forms of stimulation produced significant increases in EGFP-αCaMKII-A₂₀ mRNA translation (FIG. 4C-D). In all cases, concurrent treatment with U0126 significantly attenuated the effects of stimulation.

We next examined the role of the cis-acting elements in the αCaMKII 3′ UTR in translational induction. Neuronal activity-induced stimulation of reporter mRNA translation and its sensitivity to inhibition by U0126 persisted after mutation of the CPEs (FIG. 4E-F) and/or the hexamer (AAUAAA) sequence (FIG. 4G). These observations indicate that cytoplasmic polyadenylation is not essential for neuronal activity-induced ERK-dependent translation, since identical mutations in either of these elements have been shown to prevent cytoplasmic polyadenylation (Mendez and Richter, 2001). Consistent with this interpretation, translation of reporter mRNAs with differing poly(A)-tail lengths exhibited comparable sensitivity to U0126 (unpublished observations). Parallel analysis by in situ hybridization revealed no detectable differences in reporter mRNA stability or localization, transfection efficiency or neuronal survival (FIG. 4H), arguing that the observed effects are specific to translation.

### Example 6

### ERK Signaling Mediates Induction of Neuron-Wide and Local Protein Synthesis through Regulation of Multiple Translation Initiation Factors

Materials and Methods

Metabolic Pulse Labeling. Hippocampal neurons (DIV8) were preincubated in sulfur-free MEM for 1 hr. prior to stimulation. ³⁵S-methionine (0.2 mCi/mL) was added to the culture medium at the onset of stimulation. Synaptoneurosomes were incubated for 30 min. at 37° C. in Tyrode solution supplemented with ³⁵S-methionine (0.2 mCi/mL), protease inhibitors and RNase inhibitor. Hippocampal slices were perfused with aCSF supplemented with ³⁵S-methionine (1 mCi/ml) for 30 min. after delivery of the last tetanus. Pulse labeling was conducted in the presence of actinomycin-D (Calbiochem, 40 μM) and chloramphenicol (Sigma, 200 μg/ml). Equal amounts of protein from each sample were subjected to SDS-PAGE and transferred to nitrocellulose membranes. Autoradiography was performed on the dried membranes Staining with Ponceau-S confirmed equal loading.

Synaptoneurosomes. Synaptoneurosomes were prepared from cultured hippocampal neurons by passage through PTFE filters (Millipore) of decreasing pore size, as previously described (Scheetz et al., 2000).

Results

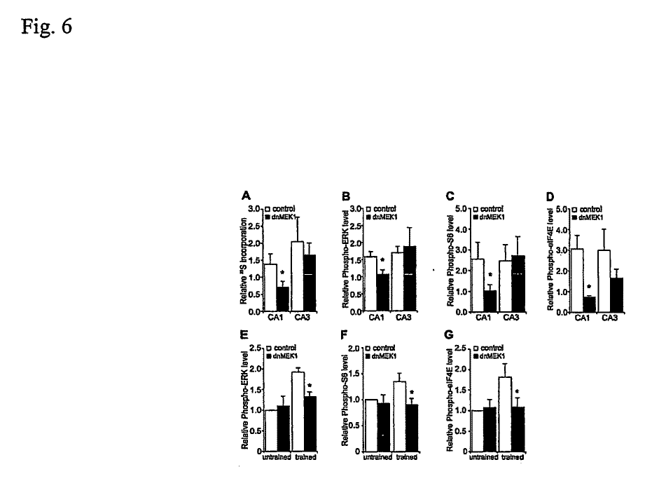

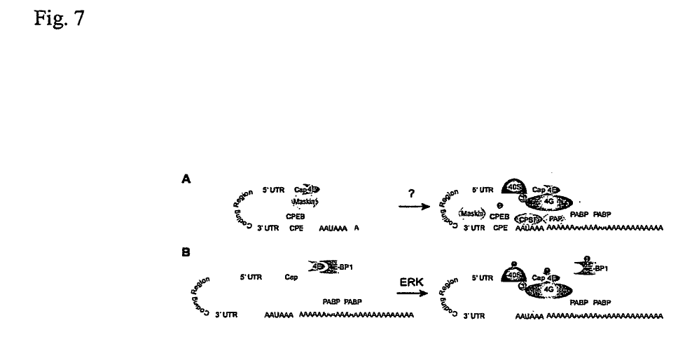

In the above analysis, reporter mRNAs remained responsive to ERK-dependent translational stimulation despite mutation of the CPE and hexamer sequences. This mutant reporter contains no other known cis-acting elements in the short 5′ and 3′ UTR segments and hence represents a “generic” mRNA. Thus, ERK signaling may provide a general pathway regulating translation of the majority of neuronal mRNAs. To test this prediction, we conducted metabolic pulse labeling in cultured hippocampal neurons. The presence of actinomycin-D and chloramphenicol precluded any confounding effects of transcription or mitochondrial translation. As shown in FIG. 5A-B, the results were remarkably similar to those obtained with transfected reporter mRNAs. Bulk translation of endogenous transcripts in response to spontaneous neuronal activity, BDNF and bicuculline was strongly inhibited by U0126 in each case. Similar levels of inhibition were obtained with the structurally distinct MEK inhibitor PD98059 (data not shown). Electrophoretic separation of radiolabeled translation products confirmed that ERK-dependent stimulation was not limited to one or few predominant protein species, but rather extended across the entire range of resolved molecular weights (FIG. 5A). The inhibitory effect of rapamycin was comparable to that of U0126, supporting a dual requirement for ERK and mTOR signaling in neuronal activity-dependent translation.

These results indicate that ERK-dependent translational modulation is a general rather than gene-specific phenomenon, suggesting that the relevant target(s) of the ERK pathway may reside in the general translation machinery. Inducible phosphorylation of specific residues in the cap-binding factor eIF4E (Ser209), its inhibitor 4E-BP (Ser65) and ribosomal protein S6 (Ser235/236) are associated with enhanced rates of translation initiation in mitotic cells (Raught et al., 2000). In parallel with metabolic labeling studies, we therefore performed Western analysis with antisera specific for the phosphorylated forms of these translation factors. Specific phosphorylation of S6, eIF4E and 4E-BP occurred in response to multiple forms of neuronal activity, and phosphorylation was significantly inhibited by U0126 treatment in each case (FIG. 5D-F). Phosphorylation of all three translation factors under conditions of spontaneous neuronal activity was also significantly inhibited by rapamycin, with rapamycin exerting the stronger effect on S6 phosphorylation, and U0126 exerting relatively stronger effects on eIF4E and 4E-BP phosphorylation. Analysis of phospho-ERK levels confirmed that ERK phosphorylation was stimulated by neuronal activity and entirely abolished by U0126 treatment (FIG. 5C).

The results described above establish an important role for ERK signaling in neuron-wide translational processes. In order to determine whether ERK signaling might also be important in protein synthesis in the vicinity of synaptses, we examined the ERK-dependence of protein synthesis and translation factor phosphorylation in synaptoneurosomes prepared from hippocampal neurons under conditions of spontaneous activity. Translation of endogenous synaptodendritic mRNAs and phosphorylation of ERK, S6 and eIF4E in synaptoneurosomes were all significantly reduced by U0126 treatment (FIG. 5G-K), indicating a similarly important role for the ERK pathway in synaptodendritic protein synthesis.

### Example 7

### Inhibiting ERK Signaling Impairs Translational Induction during L-LTP and Hippocampal Memory Formation in dnMEK1 Mice

Materials and Methods See Examples 1 and 6.

Results

To confirm the relevance of ERK-dependent translational regulation to the phenotypes observed in dnMEK1 mice, we analyzed translational activity in the context of hippocampal L-LTP and memory formation. First, we assessed changes in translational activity occurring in areas CA1 and CA3 of control and mutant hippocampal slices in response to the pattern of repeated tetanization used to induce L-LTP. Given the restricted hippocampal expression pattern of the dnMEK1 transgene, analysis of area CA3 of mutant slices provides an internal control for the specificity of transgene-dependent inhibition in area CA1. Metabolic pulse labeling in the presence of actinomycin-D revealed increased translation in both areas CA1 and CA3 of control slices following tetanization in the CA1 stratum radiatum, relative to slices that received only low-frequency stimulation (FIG. 6A). In contrast, while the same tetanic stimulation induced ³⁵S-methionine incorporation in area CA3 of mutant slices, it failed to do so in area CAL Electrophoretic separation revealed decreased labeling across the entire range of resolved molecular weights in mutant slices (data not shown). Stimulation of ERK phosphorylation following tetanization was also selectively abolished in area CA1 of mutant slices, indicating that the translational blockade in area CA1 of mutant slices correlates with a corresponding blockade of ERK activation (FIG. 6B). The stimulation of ERK phosphorylation and translation observed in both areas CA1 and CA3 of control slices presumably reflects the ability of tetanic stimulation in the CA1 stratum radiatum to induce both SC-CA1 and C/A-CA3 LTP through activation of the Schaeffer collateral and commissural/associational projections of CA3 neurons, respectively (Chattarji et al., 1989; Williams and Johnston, 1996).

We next addressed whether neuronal activity-induced ERK-dependent protein synthesis in the context of L-LTP operates via regulation of the translation initiation process. Specific phosphorylation of the translation factors S6 and eIF4E in mutant slices following tetanic stimulation exhibited a similar selective pattern of inhibition; phosphorylation of both factors in mutant slices was stimulated to control levels in area CA3, but was not stimulated in area CA1 (FIG. 6C-D). These observations suggest that ERK signaling regulates the translational events required for long-lasting synaptic plasticity in the adult hippocampus, and provide further evidence that a translational defect underlies the L-LTP deficit in dnMEK1 mice.

We next examined translational activity in the hippocampus of control and dnMEK1 mice during hippocampal memory formation. The results described above indicate that eIF4E and S6 phosphorylation provide a reliable index of translational efficiency. We therefore monitored the phosphorylation of ERK, eIF4E and S6 in hippocampal homogenates prepared from control and dnMEK1 mice 30 minutes following either contextual fear conditioning or exposure to the experimental environment without conditioning. Increases in the specific phosphorylation of ERK, S6 and eIF4E were observed as a consequence of fear conditioning in control mice, and these increases were significantly reduced in dnMEK1 mice (FIG. 6E-G). These observations further support the hypothesis that a translational defect contributes to the selective impairment in memory consolidation in dnMEK1 mice.

### Example 8

### Activation of Diverse Pathways Increases Translation in Hippocampal Neurons

Materials and Methods. Preparation of hippocampal cultures, measurement of translation, and measurement of eIF4E and S6 phosphorylation were performed as described above.

Results

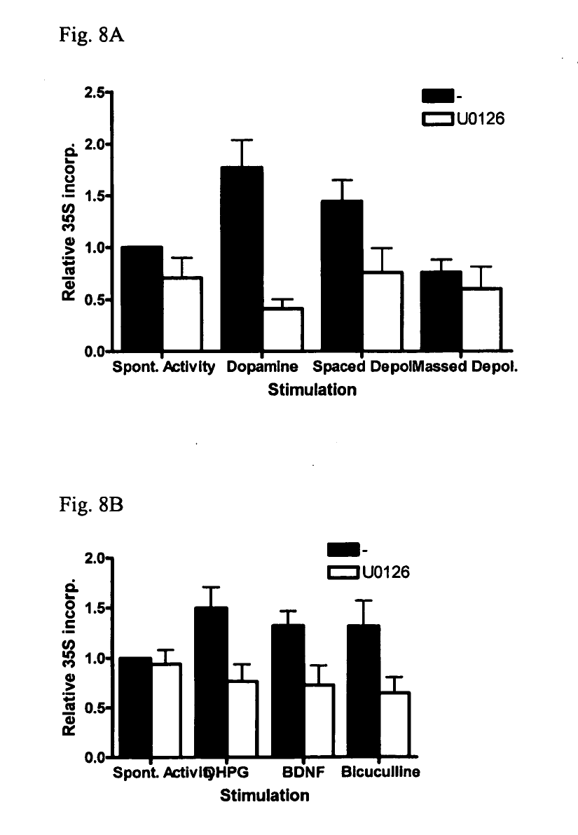

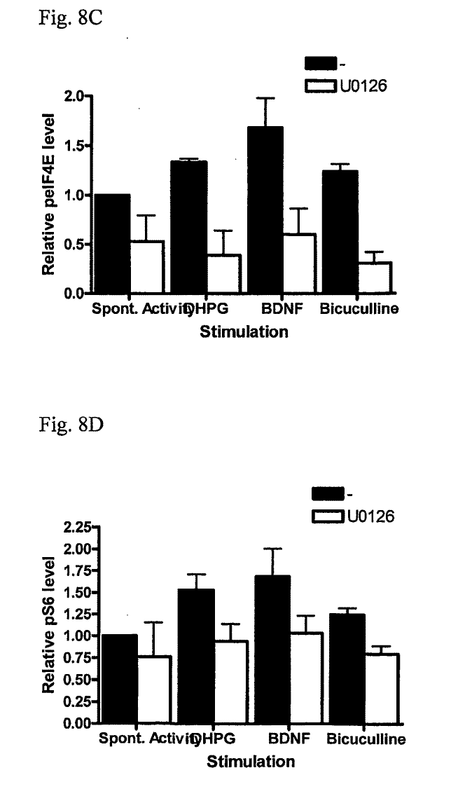

We exposed hippocampal neuronal cultures to a number of agents and known to activate a diverse set of signal transduction pathways and performed metabolic labeling with ³⁵S to measure their effect on translation. We first measured the effect of dopamine, which PKA pathway following binding to dopamine receptors D1 and D5. We compared the effect of dopamine with that of spaced depolarization, which is known to act on the MAPK pathway. As shown in FIG. 8A, dopamine caused a 77%+/−26% increase in translation levels, and this increase is blocked by the MEK inhibitor U0126. Spaced depolarization increases translation by 44%+/−20%, and this increase is also blocked by U0126.

We next examined the effect of the mGluR type I agonist DHPG and found that this agent increases translation rates in hippocampal cultured cells by 50%+/−20%, and this increase is also blocked by U0126 (FIG. 8B). Similar results were obtained with the GABA receptor antagonist bicucculine and brain derived neurotrophic factor (BDNF). Western Blot analysis showed that phosphorylation of eIF4E (FIG. 8C) and S6 (FIG. 8D) are also increased by DHPG treatment in a MEK-dependent manner (33%+/−3% and 52%+/−18% respectively). Stimulation of the adenyl cyclase/PKA pathway by exposing neurons to Sp-cAMPS also resulted in increased translation (data not shown). These results indicate that a broad spectrum of agents, acting via a diverse set of pathways, can cause an increase in neuronal translation. These results demonstrate that diverse stimuli all recruit enhanced translation, suggesting that this effect is important for their mechanism of action. In addition, the data shows that these agents all activate translation through the ERK pathway since the effect is blocked in all cases by U0126. Thus, diverse stimuli converge on the translational machinery through a common pathway, i.e. the ERK pathway.

### Example 9

### A Variety of Neuromodulatory Agents Increase Translation in Neurons in an ERK-dependent Manner

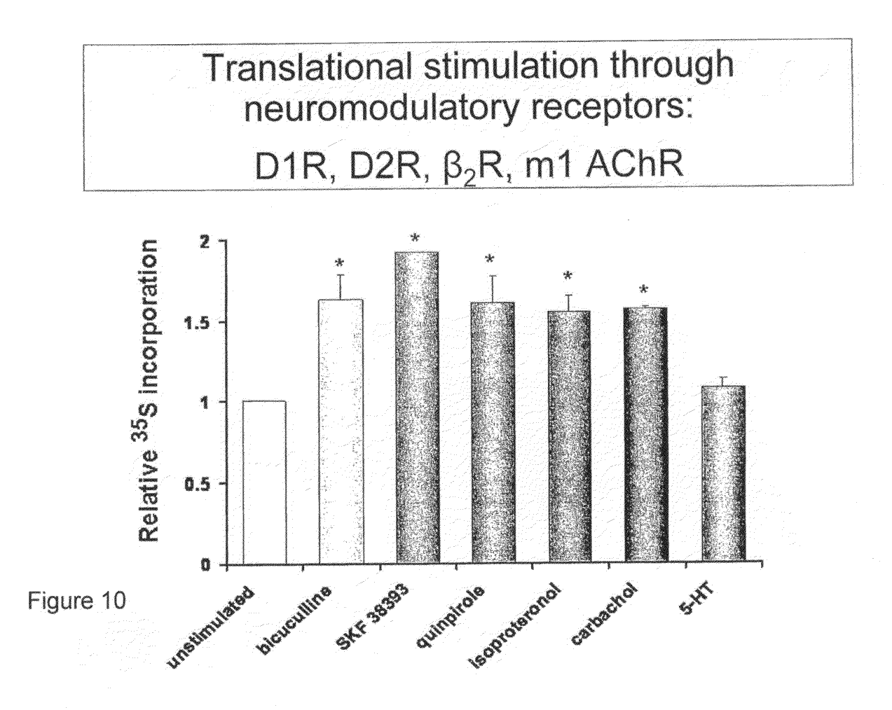

As shown in FIGS. 9 and 10, we have found that a variety of neuromodulatory agents stimulate neuronal protein synthesis in an ERK-dependent manner. Specifically, agonists of dopaminergic (SKF38393, quinpirole), beta 2 adrenergic (isoproteronol) and m1 muscarinic acetylcholine (carbachol) receptors stimulate translation of endogenous neuronal mRNAs, as indicated by enhanced metabolic labeling of newly synthesized proteins. Each agonist was employed at a final concentration of 10 uM or 50 uM, and dose-dependent sitmulations of protein synthesis were observed. In all cases, the observed increases in neuronal protein synthesis were blocked by inhibition of the ERK signaling cascade by pretreatment with the specific MEK inhibitor U0126. FIG. 9 shows results of experiments performed on hippocampal brain slices, and FIG. 10 shows results of experiments performed in cultured hippocampal neurons.

### ADDITIONAL EMBODIMENTS, EQUIVALENTS, AND SCOPE

The foregoing description is to be understood as being representative only and is not intended to be limiting. Alternative methods for modulating translation and/or MAP kinase pathways are intended to be included within the accompanying claims. In particular, compositions and methods of treatment involving drugs that act on pathways and by mechanisms discussed herein are encompassed even if not explicitly listed. One of ordinary skill in the art will readily be able to identify such known compounds by referring to, e.g., Goodman and Gilman, supra, and/or the scientific and/or patent literature.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the following claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context.

Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more limitations found in any other claim that is dependent on the same base claim. In addition, it is to be understood that any one or more embodiments or elements may be explicitly excluded from the claims even if the specific exclusion is not set forth explicitly herein.

The inclusion of a “providing a mammalian subject.” step in certain methods of the invention is intended to indicate that the composition is administered to a mammalian subject in whom modulation of cognitive function is desirable and that the composition is administered at least in part for the purpose of modulating cognitive function. Thus the subject will typically have or be at risk of developing a disease, disorder, or condition characterized by cognitive impairment and/or loss of cognitive function or whose cognitive function is recognized as being abnormal or deficient in one or more respects. The composition is administered to treat the disease, disorder, or condition and/or to ameliorate the abnormality or deficiency in cognitive function, typically upon the sound recommendation of a medical or surgical practitioner, e.g., who may or may not be the same individual who administers the composition. The invention includes embodiments in which a step of providing is not explicitly included and embodiments in which a step of providing is included. The invention also includes embodiments in which a step of identifying the subject as being in need of modulation of cognitive function is included. For example, in certain embodiments the subject is identified as being at risk of or suffering from Alzheimer's disease, age-associated memory impairment, mild cognitive impairment, trauma-associated cognitive impairment, toxin-associated cognitive impairment, or dementia. In other embodiments of the invention the subject is identified as being at risk of or suffering from mental retardation, fragile X syndrome, tuberous sclerosis, or autism.

### REFERENCES

Abel, T., Nguyen, P. V., Barad, M., Deuel, T. A., Kandel, E. R., and Bourtchouladze, R. (1997). Genetic demonstration of a role for PKA in the late phase of LTP and in hippocampus-based long-term memory. Cell 88, 615-626.
Antoniadis, E. A., and McDonald, R. J. (2000). Amygdala, hippocampus and discriminative fear conditioning to context. Behav Brain Res 108, 1-19.
Barco, A., Alarcon, J. M., and Kandel, E. R. (2002). Expression of constitutively active CREB protein facilitates the late phase of long-term potentiation by enhancing synaptic capture. Cell 108, 689-703.
Burns, A. & Zaudig, M (2002). Mild cognitive impairment in older people. The Lancet 360, 1963-1965.
Casadio, A., Martin, K. C., Giustetto, M., Zhu, H., Chen, M., Bartsch, D., Bailey, C. H., and Kandel, E. R. (1999). A transient neuron-wide form of CREB-mediated long-term facilitation can be stabilized at specific synapses by local protein synthesis. Cell 99, 221-237.
Chang, L., and Karin, M. (2001). Mammalian MAP kinase signalling cascades. Nature 410, 37-40.
Chattarji, S., Stanton, P. K., and Sejnowski, T. J. (1989). Commissural synapses, but not mossy fiber synapses, in hippocampal field CA3 exhibit associative long-term potentiation and depression. Brain Res 495, 145-150.
Clark, C. M. & Karlawish, J. H. T. (2003). Alzheimer disease: current concepts and emerging diagnostic and therapeutic strategies. Ann. Intern. Med. 138: 400-410.
Davis, H. P., and Squire, L. R. (1984). Protein synthesis and memory: a review. Psych Bull 96, 518-559.
Frankland, P. W., Cestari, V., Filipkowski, R. K., McDonald, R. J., and Silva, A. J. (1998). The dorsal hippocampus is essential for context discrimination but not for contextual conditioning. Behav Neurosci 112, 863-874.
Frey, J. U. (2001). Long-lasting hippocampal plasticity: cellular model for memory consolidation? Results Probl Cell Differ 34, 27-40.
Frey, U., Frey, S., Schollmeier, F., and Krug, M. (1996). Influence of actinomycin D, a RNA synthesis inhibitor, on long-term potentiation in rat hippocampal neurons in vivo and in vitro. J Physiol 490 (Pt 3), 703-711.
Frey, U., Krug, M., Reymann, K. G., and Matthies, H. (1988). Anisomycin, an inhibitor of protein synthesis, blocks late phases of LTP phenomena in the hippocampal CA region in vitro. Brain Res 452, 57-65.
Frey, U., and Morris, R. G. (1997). Synaptic tagging and long-term potentiation. Nature 385, 533-536.
Gingras, A. C., Raught, B., and Sonenberg, N. (2001). Regulation of translation initiation by FRAP/mTOR. Genes Dev 15, 807-826.
Godsil, B. P., Quinn, J. J., and Fanselow, M. S. (2000). Body temperature as a conditional response measure for pavlovian fear conditioning. Learn Mem 7, 353-356.
Herbert, T. P., Tee, A. R., and Proud, C. G. (2002). The extracellular signal-regulated kinase pathway regulates the phosphorylation of 4E-BP1 at multiple sites. J Biol Chem 277, 11591-11596.
Houx, P., et al., (2002) Testing cognitive function in elderly populations: the PROSPER study, Journal of Neurology Neurosurgery and Psychiatry 73:385-389.
Huang, Y. Y., and Kandel, E. R. (1994). Recruitment of long-lasting and protein kinase A-dependent long-term potentiation in the CA 1 region of hippocampus requires repeated tetanization. Learn Mem 1, 74-82.
Impey, S., Obrietan, K., and Storm, D. R. (1999). Making new connections: role of ERK/MAP kinase signaling in neuronal plasticity. Neuron 23, 11-14.
Jin, P., and Warren, S. T. (2003). New insights into fragile X syndrome: from molecules to neurobehaviors. Trends Biochem Sci 28, 152-158.
Kandel, E. R. (2001). The molecular biology of memory storage: a dialogue between genes and synapses. Science 294, 1030-1038.
Kang, H., Sun, L. D., Atkins, C. M., Soderling, T. R., Wilson, M. A., and Tonegawa, S. (2001). An important role of neural activity-dependent CaMKIV signaling in the consolidation of long-term memory. Cell 106, 771-783.
Kim, J. J., and Fanselow, M. S. (1992). Modality-specific retrograde amnesia of fear. Science 256, 675-677.
Kwiatkowski, D. J., and Manning, B. D. (2005). Tuberous sclerosis: a GAP at the crossroads of multiple signaling pathways. Hum Mol Genet. 14 Spec No. 2, R251-258.
Lakso, M., Sauer, B., Mosinger, B., Jr., Lee, E. J., Manning, R. W., Yu, S. H., Mulder, K. L., and Westphal, H. (1992). Targeted oncogene activation by site-specific recombination in transgenic mice. Proc Natl Acad Sci USA 89, 6232-6236.
Liu, G., and Tsien, R. W. (1995). Synaptic transmission at single visualized hippocampal boutons. Neuropharmacology 34, 1407-1421.
Ma, L., Chen, Z., Erdjument-Bromage, H., Tempst, P., and Pandolfi, P. P. (2005). Phosphorylation and functional inactivation of TSC2 by Erk implications for tuberous sclerosis and cancer pathogenesis. Cell 121, 179-193.
Mansour, S. J., Matten, W. T., Hermann, A. S., Candia, J. M., Rong, S., Fukasawa, K., Vande Woude, G. F., and Ahn, N. G. (1994). Transformation of mammalian cells by constitutively active MAP kinase kinase. Science 265, 966-970.
Martin, K. C., Barad, M., and Kandel, E. R. (2000). Local protein synthesis and its role in synapse-specific plasticity. Curr Opin Neurobiol 10, 587-592.
Mathews, M. B., Sonenberg, N., and Hershey, J. W. B. (2000). Origins and Principles of Translational Control. In Translational Control of Gene Expression, N. Sonenberg, Hershey, J. W. B. and Mathews, M. B., ed. (Cold Spring Harbor, N.Y., Cold Spring Harbor Press), pp. 1-31.
Mazzucchelli, C., and Brambilla, R. (2000). Ras-related and MAPK signalling in neuronal plasticity and memory formation. Cell Mol Life Sci 57, 604-611.
Mazzucchelli, C., Vantaggiato, C., Clamei, A., Fasano, S., Pakhotin, P., Krezel, W., Welzl, H., Wolfer, D. P., Pages, G., Valverde, O., et al. (2002). Knockout of ERK1 MAP kinase enhances synaptic plasticity in the striatum and facilitates striatal-mediated learning and memory. Neuron 34, 807-820.
McAllister, A. K., Katz, L. C., and Lo, D. C. (1999). Neurotrophins and synaptic plasticity. Annu Rev Neurosci 22, 295-318.
McGaugh, J. L. (2000). Memory—a century of consolidation. Science 287, 248-251.
Mendez, R., and Richter, J. D. (2001). Translational control by CPEB: a means to the end. Nat Rev Mol Cell Biol 2, 521-529.
Meyuhas, O. (2000). Synthesis of the translational apparatus is regulated at the translational level. Eur J Biochem 267, 6321-6330.
Morris, R. G. M., Garrud, P., Rawlins, J. N. P., and O'Keefe, J. (1982). Place navigation impaired in rats with hippocampal lesions. Nature 297, 681-683.
Nguyen, P. V., Abel, T., and Kandel, E. R. (1994). Requirement of a critical period of transcription for induction of a late phase of LTP. Science 265, 1104-1107.
Pearson, G., Robinson, F., Beers Gibson, T., Xu, B. E., Karandikar, M., Berman, K., and Cobb, M. H. (2001). Mitogen-activated protein (MAP) kinase pathways: regulation and physiological functions. Endocr Rev 22, 153-183.
Petersen, R. C., Doody, R., Kurz, A., Mohs, R. C., Morris, J. C., Rabins, P. V., Ritchie, K., Rossor, M., Thal, L. & Winblad, B. (2001). Current Concepts in Mild Cognitive Impairment. Arch. Neurol. 58, 1985-1992.
Phillips, A. G., and LeDoux, J. E. (1992). Differential contribution of amygdala and hippocampus to cued and contextual fear conditioning. Behav Neurosci 106, 274-285.
Raught, B., Gingras, A.-C., and Sonenberg, N. (2000). Regulation of Ribosomal Recruitment in Eukaryotes. In Translational Control of Gene Expression, N. Sonenberg, Hershey, J. W. B. and Mathews, M. B., ed. (Cold Spring Harbor, N.Y., Cold Spring Harbor Press), pp. 245-293.
Reisberg, B., Doody, R. Stoffler, A., Scmitt, A., Feris, S., Mobius, H. J., Memantine study group. (2003) Memantine in moderate-to-severe Alzheimer's disease. N Engl J Med. 348(14):1333-41
Richter, J. D., and Lorenz, L. J. (2002). Selective translation of mRNAs at synapses. Curr Opin Neurobiol 12, 300-304.
Schafe, G. E., Nadel, N. V., Sullivan, G. M., Harris, A., and LeDoux, J. E. (1999). Memory consolidation for contextual and auditory fear conditioning is dependent on protein synthesis, PKA, and MAP kinase. Learn Mem 6, 97-110.
Soderling, T. R. (2000). CaM-kinases: modulators of synaptic plasticity. Curr Opin Neurobiol 10, 375-380.
Steward, O., and Schuman, E. M. (2001). Protein synthesis at synaptic sites on dendrites. Annu Rev Neurosci 24, 299-325.
Sweatt, J. D. (2001). The neuronal MAP kinase cascade: a biochemical signal integration system subserving synaptic plasticity and memory. J Neurochem 76, 1-10.
Tang, S. J., Reis, G., Kang, H., Gingras, A. C., Sonenberg, N., and Schuman, E. M. (2002). A rapamycin-sensitive signaling pathway contributes to long-term synaptic plasticity in the hippocampus. Proc Natl Acad Sci USA 99, 467-472.
Treisman, R. (1996). Regulation of transcription by MAP kinase cascades. Curr Opin Cell Biol 8, 205-215.
Tsien, J. Z., Chen, D. F., Gerber, D., Tom, C., Mercer, E. H., Anderson, D. J., Mayford, M., Kandel, E. R., and Tonegawa, S. (1996). Subregion- and cell type-restricted gene knockout in mouse brain. Cell 87, 1317-1326.
Tyler, W. J., Alonso, M., Bramham, C. R., and Pozzo-Miller, L. D. (2002). From acquisition to consolidation: on the role of brain-derived neurotrophic factor signaling in hippocampal-dependent learning. Learn Mem 9, 224-237.
Wang, X., Flynn, A., Waskiewicz, A. J., Webb, B. L., Vries, R. G., Baines, I. A., Cooper, J. A., and Proud, C. G. (1998). The phosphorylation of eukaryotic initiation factor eIF4E in response to phorbol esters, cell stresses, and cytokines is mediated by distinct MAP kinase pathways. J Biol Chem 273, 9373-9377.
Waskiewicz, A. J., Flynn, A., Proud, C. G., and Cooper, J. A. (1997). Mitogen-activated protein kinases activate the serine/threonine kinases Mnk1 and Mnk2. Embo J 16, 1909-1920.
West, A. E., Chen, W. G., Dalva, M. B., Dolmetsch, R. E., Kornhauser, J. M., Shaywitz, A. J., Takasu, M. A., Tao, X., and Greenberg, M. E. (2001). Calcium regulation of neuronal gene expression. Proc Natl Acad Sci USA 98, 11024-11031.
Williams, S. H., and Johnston, D. (1996). Actions of endogenous opioids on NMDA receptor-independent long-term potentiation in area CA3 of the hippocampus. J Neurosci 16, 3652-3660.
Winder, D. G., Mansuy, I. M., Osman, M., Moallem, T. M., and Kandel, E. R. (1998). Genetic and pharmacological evidence for a novel, intermediate phase of long-term potentiation suppressed by calcineurin. Cell 92, 25-37.
Wong, S. T., Athos, J., Figueroa, X. A., Pineda, V. V., Schaefer, M. L., Chavkin, C. C., Muglia, L. J., and Storm, D. R. (1999). Calcium-stimulated adenylyl cyclase activity is critical for hippocampus-dependent long-term memory and late phase LTP. Neuron 23, 787-798.
Wu, G. Y., Deisseroth, K., and Tsien, R. W. (2001). Spaced stimuli stabilize MAPK pathway activation and its effects on dendritic morphology. Nat Neurosci 4, 151-158.
Wu, L., Wells, D., Tay, J., Mendis, D., Abbott, M. A., Barnitt, A., Quinlan, E., Heynen, A., Fallon, J. R., and Richter, J. D. (1998). CPEB-mediated cytoplasmic polyadenylation and the regulation of experience-dependent translation of alpha-CaMKII mRNA at synapses. Neuron 21, 1129-1139.
Xia, Z., Dickens, M., Raingeaud, J., Davis, R. J., and Greenberg, M. E. (1995). Opposing effects of ERK and JNK-p38 MAP kinases on apoptosis. Science 270, 1326-1331.
Zeng, H., Chattarji, S., Barbarosie, M., Rondi-Reig, L., Philpot, B. D., Miyakawa, T., Bear, M. F., and Tonegawa, S. (2001). Forebrain-specific calcineurin knockout selectively impairs bidirectional synaptic plasticity and working/episodic-like memory. Cell 107, 617-629.
Zhang H, Cicchetti G, Onda H, Koon H B, Asrican K, Bajraszewski N, Vazquez F, Carpenter C L, Kwiatkowski D J. (2003) Loss of Tsc1/Tsc2 activates mTOR and disrupts PI3K-Akt signaling through downregulation of PDGFR J Clin Invest. 112(8):1223-33.

We claim:

1. A method of modulating cognitive function in a mammalian subject comprising steps of:
(a) providing a mammalian subject in need of modulation of cognitive function; and
(b) administering to the subject a composition comprising an agent that modulates translation.

2. The method of claim 1, wherein the agent increases translation so that cognitive function is enhanced.

3. The method of claim 2, wherein the agent enhances long-term memory.

4. The method of claim 1, wherein the agent modulates the activity or abundance, or both, of a component of the general translation machinery.

5. The method of claim 4, wherein the component is a translation initiation factor.

6. The method of claim 1, wherein the component is selected from the group consisting of: ribosomal protein S6, eIF4E, and a 4E-BP.

7. The method of claim 1, wherein the agent modulates activity or abundance of a component of a MAPK signaling pathway.

8. The method of claim 7, wherein the agent increases activity or abundance of a component of a MAPK signaling pathway.

9. The method of claim 7, wherein the component of the MAPK signaling pathway is selected from the group consisting of: tyrosine kinase receptors, G protein coupled receptors, PI3 kinase, protein kinase C, protein kinase A, phospholipase B, adenylyl cyclase, CaM kinase II, CaM kinase IV, Src, β-arrestin, β-adrenergic receptors, Src-like protein, RasGEF SOS, Ras GAP, Ras GRP, Ras GRF, SynGap, EPAC, Ras, c-Raf, B-raf, MEK1, and MEK2.

10. The method of claim 1, wherein the agent modulates translation in the hippocampus.

11. The method of claim 1, wherein the composition comprises an agent selected from the group consisting of: a tyrosine kinase receptor agonist, a G protein coupled receptor agonist; a metabotropic glutamate receptor agonist; an NMDA receptor agonist; a GABA receptor antagonist; an ERK pathway activator; an adenylyl cyclase activator; a protein kinase A activator; and a phosphodiesterase inhibitor, and wherein the composition enhances cognitive function.

12. The method of claim 11, wherein the G protein coupled receptor is selected from the group consisting of: a beta-adrenergic receptor, an alpha-adrenergic receptor, a dopamine D1 or D5 receptor.

13. The method of claim 1, wherein the composition comprises an agent that activates the mTOR pathway, and wherein the agent enhances cognitive function.

14. The method of claim 1, wherein the composition comprises an agent that inhibits a negative regulator of the mTOR pathway, and wherein the agent enhances cognitive function.

15. The method of claim 1, wherein the composition comprises an agent that inhibits a component selected from the group consisting of: PTEN phosphatase, TSC1 and TSC2.

16. The method of claim 1, wherein the composition comprises an agent that inhibits expression or activity of a negative regulator of translation, and wherein the composition enhances cognitive function.

17. The method of claim 16, wherein the negative regulator is a 4E-BP.

18. The method of claim 16, wherein the agent is an RNAi agent.

19. The method of claim 1, wherein the composition does not modulate transcription.

20. The method of claim 1, wherein the agent modulates translation in a non gene-specific manner.

21. The method of claim 1, wherein the agent modulates translation in a gene-specific manner.

22. The method of claim 1, wherein the composition modulates translation of at least 10% of the mRNA species in a neuron.

23. The method of claim 1, wherein the composition modulates translation of at least 25% of the mRNA species in a neuron.

24. The method of claim 1, wherein the composition modulates translation of at least 75% of the mRNA species in a neuron.

25. The method of claim 1, wherein the modulation of translation is not spatially restricted to dendrites or synapses.

26. The method of claim 1, wherein the cognitive function is long-term memory.

27. The method of claim 1, wherein the cognitive function is memory formation.

28. The method of claim 1, wherein the cognitive function is memory consolidation.

29. The method of claim 1, wherein the subject is at risk of or suffering from a condition selected from the group consisting of: memory impairment, dementia, cognitive deficit, or attention deficit disorder.

30. The method of claim 1, wherein the subject is at risk of or suffering from a disease, disorder, or condition selected from the group consisting of: Alzheimer's disease, age-associated memory impairment, or mild cognitive impairment.

31. The method of claim 1, wherein the subject has suffered a traumatic brain injury or stroke.

32. The method of claim 1, wherein the subject is at risk of or suffering from a disease, disorder, or condition selected from the group consisting of: fragile X syndrome, tuberous sclerosis, or autism and the composition inhibits or decreases translation.

33. The method of claim 1, further comprising the step of:
(iii) stimulating neuronal activity in the subject's CNS.

34. The method of claim 33, wherein the step of stimulating comprises: engaging the subject in a program of rehabilitation that stimulates neuronal activity, wherein the subject is so engaged during at least part of the time interval during which the agent is administered or during which the agent remains active in the nervous system of the subject.

35. A method of modulating cognitive function in a mammalian subject comprising steps of:
(a) providing a mammalian subject in need of modulation of cognitive function; and
(b) administering to the subject a composition that modulates a MAPK pathway.

36. The method of claim 35, wherein the MAPK signaling pathway is an ERK signaling pathway.

37. The method of claim 35, wherein the subject is in need of cognitive enhancement and the composition increases activity of a MAP kinase.

38. The method of claim 35, wherein the composition comprises an agent that modulates translation in a non gene-specific manner.

39. The method of claim 35, wherein the composition comprises an agent selected from the group consisting of: a tyrosine kinase receptor agonist, a G protein coupled receptor agonist; a metabotropic glutamate receptor agonist; an NMDA receptor agonist; a GABA receptor antagonist; an ERK pathway activator; an adenylyl cyclase activator; a protein kinase A activator; and a phosphodiesterase inhibitor, and wherein the composition enhances cognitive function.

40. The method of claim 35, further comprising the step of:
(iii) stimulating neuronal activity in the subject's CNS.

41. The method of claim 40, wherein the step of stimulating comprises: engaging the subject in a program of rehabilitation that stimulates neuronal activity, wherein the subject is so engaged during at least part of the time interval during which the agent is administered or during which the agent remains active in the nervous system of the subject.

42. A method of modulating cognitive function in a mammalian subject comprising steps of:
(a) providing a mammalian subject in need of modulation of cognitive function; and
(b) administering to the subject a composition that modulates the mTOR pathway.

43. The method of claim 42, wherein the subject is in need of cognitive enhancement and the composition increases activity of the mTOR pathway.

44. The method of claim 42, wherein the composition comprises an agent that modulates the activity or abundance of a component selected from the group consisting of: PI3K, PDK1, Akt, mTOR, p70 S6 kinase 1, and p70 S6 kinase 2.

45. The method of claim 42, wherein the composition comprises an agent that inhibits a component selected from the group consisting of: PTEN phosphatase, TSC1 and TSC2.

46. The method of claim 42, further comprising the step of:
(c) stimulating neuronal activity in the subject's CNS.

47. The method of claim 46, wherein the step of stimulating comprises: engaging the subject in a program of rehabilitation that stimulates neuronal activity, wherein the subject is so engaged during at least part of the time interval during which the agent is administered or during which the agent remains active in the nervous system of the subject.

48. A method of identifying an agent that modulates cognitive function comprising:
(a) providing a biological system for detecting an increase or decrease in translation;
(b) contacting the system with a candidate agent;
(c) determining whether the agent increases or decreases translation; and
(d) identifying the agent as a candidate modulator of cognitive function if translation is increased or decreased.

49. The method of claim 48, wherein the biological system is a cell-free translation system.

50. The method of claim 48, wherein the biological system is a neuronal culture.

51. The method of claim 48, wherein the culture comprises hippocampal neurons.

52. The method of claim 48, wherein the biological system is a hippocampal slice.

53. The method of claim 48, wherein the biological system comprises a MAP kinase.

54. The method of claim 53, wherein the MAP kinase is an ERK.

55. The method of claim 48, wherein the biological system comprises a MAP kinase, a MEK, and a MEKK.

56. The method of claim 48, further comprising the steps of:
determining whether the agent increases or decreases L-LTP in a hippocampal slice, in an animal, or both.

57. The method of claim 56, further comprising the step of:
(a) administering the agent to an animal; and
(b) subjecting the animal to a behavioral test that assesses cognition, long-term memory, or both.

58. The method of claim 48, further comprising the step of:
(a) administering the agent to an animal; and
(b) subjecting the animal to a behavioral test that assesses cognition, long-term memory, or both.

59. A method of treating a subject in need of modulation of cognitive function comprising steps of:
(a) providing a subject in need of modulation of cognitive function; and
(b) administering an agent identified according to the method of claim 48 to the subject.

60. The method of claim 59, wherein the cognitive function is long-term memory.

61. A method of identifying an agent that modulates cognitive function comprising:
(a) providing a biological system for detecting an increase or decrease in activity of a MAP kinase;
(b) contacting the system with a candidate agent;
(c) determining whether the agent increases or decreases activity of the MAP kinase; and
(d) identifying the agent as a candidate modulator of cognitive function if activity of the MAP kinase is increased or decreased.

62. The method of claim 61, wherein the biological system is a cell-free translation system.

63. The method of claim 61, wherein the biological system is a neuronal culture.

64. The method of claim 61, wherein the culture comprises hippocampal neurons.

65. The method of claim 61, wherein the biological system is a hippocampal slice.

66. The method of claim 61, further comprising the steps of:
determining whether the agent increases or decreases L-LTP in a hippocampal slice, in an animal, or both.

67. The method of claim 66, further comprising the step of:
(a) administering the agent to an animal; and
(b) subjecting the animal to a behavioral test that assesses cognition, long-term memory, or both.

68. The method of claim 61, further comprising the step of:
(a) administering the agent to an animal; and
(b) subjecting the animal to a behavioral test that assesses cognition, long-term memory, or both.

69. A method of treating a subject in need of modulation of cognitive function comprising steps of:
(a) providing a subject in need of modulation of cognitive function; and
(b) administering an agent identified according to the method of claim 61 to the subject.

70. The method of claim 69, wherein the cognitive function is long-term memory.

71. A method of identifying an agent that modulates cognitive function comprising:
(a) providing a biological system for detecting an increase or decrease in activity of a MAP kinase pathway component;
(b) contacting the system with a candidate agent;
(c) determining whether the agent increases or decreases activity of the MAP kinase pathway component; and
(d) identifying the agent as a candidate modulator of cognitive function if activity of the MAP kinase pathway component is increased or decreased.

72. The method of claim 71, wherein the biological system is a cell-free translation system.

73. The method of claim 71, wherein the biological system is a neuronal culture.

74. The method of claim 71, wherein the culture comprises hippocampal neurons.

75. The method of claim 71, wherein the biological system is a hippocampal slice.

76. The method of claim 71, wherein the biological system comprises a MAP kinase.

77. The method of claim 76, wherein the MAP kinase is an ERK.

78. The method of claim 71, wherein the biological system comprises a MAP kinase, a MEK, and a MEKK.

79. The method of claim 71, further comprising the steps of:
determining whether the agent increases or decreases L-LTP in a hippocampal slice, in an animal, or both.

80. The method of claim 79, further comprising the step of:
(a) administering the agent to an animal; and
(b) subjecting the animal to a behavioral test that assesses cognition, long-term memory, or both.

81. The method of claim 71, further comprising the step of:
(a) administering the agent to an animal; and
(b) subjecting the animal to a behavioral test that assesses cognition, long-term memory, or both.

82. The method of claim 71, wherein the MAP kinase pathway component is selected from the group consisting of: tyrosine kinase receptors, G protein coupled receptors, PI3 kinase, protein kinase C, protein kinase A, phospholipase B, adenylyl cyclase, CaM kinase II, CaM kinase IV, Src, β-arrestin, β-adrenergic receptors, Src-like protein, RasGEF SOS, Ras GAP, Ras GRP, Ras GRF, SynGap, EPAC, Ras, c-Raf, B-raf, MEK1, and MEK2.

83. The method of claim 71, wherein the agent is identified as a candidate enhancer of cognitive function if activity of the component is increased.

84. The method of claim 71, wherein the agent is identified as a candidate enhancer of cognitive function if activity of the component is decreased.

85. A method of treating a subject in need of modulation of cognitive function comprising steps of:
(a) providing a subject in need of modulation of cognitive function; and
(b) administering an agent identified according to the method of claim 71 to the subject.

86. The method of claim 85, wherein the cognitive function is long-term memory.

87. A method of identifying an agent that modulates cognitive function comprising:
(a) providing a biological system for detecting an increase or decrease in activity of regulator of a component of the general translation machinery;
(b) contacting the system with a candidate agent;
(c) determining whether the agent increases or decreases activity of a regulator of a component of the general translation machinery; and
(d) identifying the agent as a candidate modulator of cognitive function if activity of a regulator of a component of the general translation machinery is increased or decreased.

88. The method of claim 87, wherein the regulator is selected from the group consisting of: insulin receptor, PI3 kinase, PTEN phosphatase, PKD1, PKD2, Akt, PKCγ, P38 MAPK, ERK 1, ERK2, MNK, mTOR, PKCδ, Raptor, S6K1, S6K2, PP2A, TSC1, TSC2, ATM, MNK1, and MNK2.

89. The method of claim 87, wherein the agent is identified as an enhancer of cognitive function if activity of the regulator is increased.

90. The method of claim 87, wherein the agent is identified as a candidate enhancer of cognitive function if activity of the regulator is decreased.

91. The method of claim 87, wherein the component is selected from the group consisting of: eIF1A, eIF2a, eIF2b, eIF3, eIF4A, eIF4B, eIF4E, eIF4F, eIF4G, eIF5, eIF5B, PABP, 4E-BP, CPEB, MNK1, MNK2, and Maskin.

92. The method of claim 87, wherein the biological system is a cell-free translation system.

93. The method of claim 87, wherein the biological system is a neuronal culture.

94. The method of claim 87, wherein the culture comprises hippocampal neurons.

95. The method of claim 87, wherein the biological system is a hippocampal slice.

96. The method of claim 87, wherein the biological system comprises a MAP kinase.

97. The method of claim 96, wherein the MAP kinase is an ERK.

98. The method of claim 87, wherein the biological system comprises a MAP kinase, a MEK, and a MEKK.

99. The method of claim 87, further comprising the steps of:
determining whether the agent increases or decreases L-LTP in a hippocampal slice, in an animal, or both.

100. The method of claim 99, further comprising the step of:
(a) administering the agent to an animal; and
(b) subjecting the animal to a behavioral test that assesses cognition, long-term memory, or both.

101. The method of claim 87, further comprising the step of:
(a) administering the agent to an animal; and
(b) subjecting the animal to a behavioral test that assesses cognition, long-term memory, or both.

102. A method of treating a subject in need of modulation of cognitive function comprising steps of:
(a) providing a subject in need of modulation of cognitive function; and
(b) administering an agent identified according to the method of claim 87 to the subject.

103. The method of claim 102, wherein the cognitive function is long-term memory.

104. A method of identifying an agent that modulates cognitive function comprising:
(a) providing a biological system for detecting an increase or decrease in phosphorylation of a component of the general translation machinery;
(b) contacting the system with a candidate agent;
(c) determining whether the agent increases or decreases phosphorylation of the component; and
(d) identifying the agent as a candidate modulator of cognitive function if phosphorylation of the component is increased or decreased.

105. The method of claim 104, wherein the component is selected from the group consisting of: eIF4E, a 4E-BP, and S6.

106. The method of claim 104, wherein the biological system is a cell-free translation system.

107. The method of claim 104, wherein the biological system is a neuronal culture.

108. The method of claim 104, wherein the culture comprises hippocampal neurons.

109. The method of claim 104, wherein the biological system is a hippocampal slice.

110. The method of claim 104, wherein the biological system comprises a MAP kinase.

111. The method of claim 110, wherein the MAP kinase is an ERK.

112. The method of claim 104, wherein the biological system comprises a MAP kinase, a MEK, and a MEKK.

113. The method of claim 104, further comprising the steps of:
determining whether the agent increases or decreases L-LTP in a hippocampal slice, in an animal, or both.

114. The method of claim 113, further comprising the step of:
(a) administering the agent to an animal; and
(b) subjecting the animal to a behavioral test that assesses cognition, long-term memory, or both.

115. The method of claim 104, further comprising the step of:
(a) administering the agent to an animal; and
(b) subjecting the animal to a behavioral test that assesses cognition, long-term memory, or both.

116. A method of treating a subject in need of modulation of cognitive function comprising steps of:
(a) providing a subject in need of modulation of cognitive function; and
(b) administering an agent identified according to the method of claim 104 to the subject.

117. The method of claim 116, wherein the cognitive function is long-term memory.

118. A method of identifying an agent that modulates cognitive function comprising:
(a) contacting a biological system with an agent selected from the group consisting of: a tyrosine kinase receptor agonist, a G protein coupled receptor agonist or antagonist, a metabotropic glutamate receptor agonist or antagonist, an NMDA receptor agonist or antagonist, a GABA receptor agonist or antagonist, an ERK pathway activator or inhibitor, an adenylyl cyclase activator or inhibitor, a protein kinase A activator or inhibitor, and a phosphodiesterase activator or inhibitor; and
(b) determining whether the compound causes an increase or decrease in translation in the biological system; and
(c) identifying the agent as a modulator of cognitive function if translation is increased or decreased.

119. The method of claim 118, wherein the G protein coupled receptor is selected from the group consisting of: dopamine receptor D1, dopamine receptor D2, dopamine receptor D5, and β-adrenergic receptors.

120. The method of claim 118, wherein the biological system comprises cells.

121. The method of claim 118, wherein the biological system comprises neurons.

122. The method of claim 118, wherein the biological system comprises hippocampal neurons.

123. The method of claim 118, wherein the biological system comprises a hippocampal slice.

124. The method of claim 118, wherein the biological system comprises a brain region.

125. The method of claim 118, further comprising the steps of:
determining whether the agent increases or decreases L-LTP in a hippocampal slice, in an animal, or both.

126. The method of claim 118, further comprising the step of:
(a) administering the agent to an animal; and
(b) subjecting the animal to a behavioral test that assesses cognition, long-term memory, or both.

127. A method of treating a subject in need of modulation of cognitive function comprising steps of:
(a) providing a subject in need of modulation of cognitive function; and
(b) administering an agent identified according to the method The method of claim 118, to the subject.

128. The method of claim 127, wherein the cognitive function is long-term memory.

129. The method of claim 127, wherein the agent is selected from the group consisting of: a tyrosine kinase receptor agonist, a G protein coupled receptor agonist, a metabotropic glutamate receptor agonist, an NMDA receptor agonist, a GABA receptor antagonist, an ERK pathway activator, an adenylyl cyclase activator, a protein kinase A activator, and a phosphodiesterase inhibitor, and wherein the identifying step comprises identifying the agent as an enhances of cognitive function.

130. A method of identifying an agent that modulates cognitive function comprising:
(a) providing a biological system for detecting an increase or decrease in activity of a component selected from the group consisting of: a G protein coupled receptor, a receptor tyrosine kinase, a phosphatase, a phosphodiesterase, a guanine nucleotide exchange factor, a guanine nucleotide release factor, adenylyl cyclase, and a GTPase;
(b) contacting the system with a candidate agent;
(c) determining whether the agent increases or decreases activity of the component;
(d) identifying the agent as a candidate modulator of translation if activity of the component is increased or decreased;
(e) contacting a biological system comprising neurons with an agent identified in step (d) as a candidate modulator of translation;
(f) determining whether the agent causes an increase or decrease in translation; and
(g) identifying the agent as a modulator of cognitive function if the agent causes an increase or decrease in translation.

131. The method of claim 130, further comprising the steps of:
determining whether the agent increases or decreases L-LTP in a hippocampal slice, in an animal, or both.

132. The method of claim 130, further comprising the step of:
(a) administering the agent to an animal; and
(b) subjecting the animal to a behavioral test that assesses cognition, long-term memory, or both.

133. A method of treating a subject in need of modulation of cognitive function comprising steps of:
(a) providing a subject in need of modulation of cognitive function; and
(b) administering an agent identified according to the method of claim 130 to the subject.

134. The method of claim 133, wherein the cognitive function is long-term memory.

135. A method of modulating translation in a neuron comprising contacting the neuron with an agent that modulates expression or activity of a component of the MAPK signaling pathway.

136. The method of claim 135, wherein the agent enhances or activates expression or activity of a component of the MAPK signaling pathway so that translation is increased.

137. The method of claim 135, wherein the agent inhibits or represses expression or activity of a component of the MAPK signaling pathway so that translation is reduced.

138. The method of claim 135, wherein the contacting takes place in cell or tissue culture.

139. The method of claim 135, wherein the contacting takes place in a living mammalian organism.

140. The method of claim 135, wherein the component of the MAPK signaling pathway is ERK.

141. The method of claim 135, wherein the agent is selected from the group consisting of: a tyrosine kinase receptor agonist, a G protein coupled receptor agonist, a metabotropic glutamate receptor agonist, an NMDA receptor agonist, a GABA receptor antagonist, an ERK pathway activator, an adenylyl cyclase activator, a protein kinase A activator, a phosphodiesterase inhibitor, a dopamine receptor agonist, a noradrenergic receptor agonist, and a muscarinic acetylcholine receptor agonist.

142. The method of claim 135, wherein the agent is selected from the group consisting of: a metabotropic glutamate receptor antagonist; an NMDA receptor antagonist; a GABA receptor agonist, an ERK pathway inhibitor, an adenylyl cyclase inhibitor; a protein kinase A inhibitor; and a phosphodiesterase activator, a dopamine receptor agonist, a noradrenergic receptor antagonist, and a muscarinic acetylcholine receptor antagonist.

143. The method of claim 135, further comprising stimulating the neuron.
